(19) United States
(12) Patent Application Publication
(10) Pub. No.: US 2021/0012871 A1
(43) Pub. Date: Jan. 14, 2021

(54) SYSTEMS AND METHODS FOR DETERMINING COMPLIANCE AND EFFICACY OF A DOSING REGIMEN FOR A PHARMACEUTICAL AGENT

(71) Applicant: Sumner Bluffs, LLC, Tea, SD (US)

(72) Inventors: Tony A. Hagen, Sioux Falls, SD (US); Tim Hagen, Elk Grove, CA (US); Jake Zastrow, Sioux Falls, SD (US)

(21) Appl. No.: 16/789,220

(22) Filed: Feb. 12, 2020

Related U.S. Application Data
(63) Continuation of application No. 15/521,851, filed on Apr. 25, 2017, now abandoned.
(60) Provisional application No. 62/068,648, filed on Oct. 25, 2014.
(60) Provisional application No. 62/145,399, filed on Apr. 9, 2015.
(60) Provisional application No. 62/191,976, filed on Jul. 13, 2015.
(60) Provisional application No. 62/191,979, filed on Jul. 13, 2015.
(60) Provisional application No. 62/191,972, filed on Jul. 13, 2015.
(60) Provisional application No. 62/191,974, filed on Jul. 13, 2015.
(60) Provisional application No. 62/212,441, filed on Aug. 31, 2015.

(51) Int. Cl. G16H 20/10; G16H 10/60; G16H 10/20; G06Q 10/10

(52) U.S. Cl. CPC G16H 20/10; G16H 10/60; Y02A 90/10; G06Q 10/1093; G16H 10/20

(57) ABSTRACT

Embodiments of the present invention relate generally to determining compliance and/or efficacy of a dosing regimen of a pharmaceutical or other monitored agent to a subject. In certain embodiments, the present disclosure provides devices and methods for biometric data acquisition and monitoring before, during, or after administration of a pharmaceutical or other monitored agent to a user. Other embodiments of the present invention improve patient outcomes by giving patients more control over the delivery of their medication and by providing physicians with meaningful and accurate biometric and diagnostic data during treatment.

### RELATED APPLICATIONS

This application is a continuation application of U.S. Nonprovisional patent application Ser. No. 15/521,851 filed Apr. 25, 2017, which is a 371 of International Application No. PCT/US2015/056507 filed Oct. 20, 2015, which claims the benefit of U.S. Provisional Patent Application Ser. No. 62/068,648 filed Oct. 25, 2014, U.S. Provisional Patent Application Ser. No. 62/145,399 filed Apr. 9, 2015, U.S. Provisional Patent Application Ser. No. 62/191,979 filed Jul. 13, 2015, U.S. Provisional Patent Application Ser. No. 62/191,976 filed Jul. 13, 2015, U.S. Provisional Patent Application Ser. No. 62/191,972 filed Jul. 13, 2015, U.S. Provisional Patent Application Ser. No. 62/191,974, filed Jul. 13, 2015, and U.S. Provisional Patent Application Ser. No. 62/212,441 filed Aug. 31, 2015. These applications are incorporated herein by reference in their entirety for all purposes.

### FIELD

Embodiments of the present disclosure generally relate to determining compliance to a dosing regimen for a pharmaceutical agent or other regulated or prescribed agent. In certain embodiments, the present disclosure provides devices and methods for biometric data acquisition and monitoring before, during, or after administration of pharmaceutical agent or other regulated or prescribed agent to a user.

### BACKGROUND

The concept of personalized medicine is changing the healthcare landscape throughout the world. Personalized medicine is an emerging field that uses various diagnostic tools (e.g., genetic markers, biometric data) to help determine which medical treatments and procedures will be best for a given patient. By combining this personalized diagnostic information with a patient's medical records and individual needs, personalized medicine allows physicians and patients to develop targeted prevention and treatment plans. The goal of personalized medicine is to provide the right treatment in the right dose to the right patient at the right time.

Although great progress has been made, the goals of personalized medicine have not yet been fully realized. For example, there is a paucity of currently available drug delivery devices with the capability to administer safely and effectively one or more pharmaceutical agents to a patient. Prescription medications and over-the-counter drugs are administered to patients in various forms, and this typically requires a different device for each mode of administration. Additionally, physicians typically must not only rely on their patients to adhere to their medical instructions after leaving the clinical setting, they must also trust that their patients are accurately reporting information regarding their treatment. The ability for patients to have more control over the delivery of their medication, while at the same time providing physicians with meaningful and accurate biometric and diagnostic data during treatment would greatly augment the overall goals of personalized medicine and lead to better patient outcomes.

### SUMMARY

Embodiments of the present invention include but are not limited to methods for: receiving, from a pharmaceutical agent delivery and biometric data acquisition device, delivery parameters of an administered pharmaceutical agent to a user; receiving, from the pharmaceutical agent delivery and biometric data acquisition device, at least one biometric response of the user; determining, using a computing device, a compliance rating using at least one of the following: the delivery parameters of the administered pharmaceutical agent and the at least one biometric response. In some embodiments, the at least one biometric response includes at least one of the following: a galvanic skin response, a blood oxygen level response, a body temperature response, a heartrate response, a perfusion index response, a blood pressure response, a retina response, an eye movement response, an inhalation velocity response, an inhalation pressure response, an inhalation volume response, an expiratory velocity response, an expiratory pressure response, an expiratory volume response or an exhale chemical composition response. In other embodiments, the at least one biometric response to the pharmaceutical agent is measured by the pharmaceutical agent delivery and biometric data acquisition device during at least one of the following intervals: less than five minutes after taking the pharmaceutical agent, less than one hour after taking the pharmaceutical agent, less than one day after taking the pharmaceutical agent, less than one week after taking the pharmaceutical agent or less than one month after taking the pharmaceutical agent.

In certain embodiments, methods can further include receiving at least one biometric parameter for a user, wherein the at least one biometric parameter for the user is measured by the pharmaceutical agent delivery and biometric data acquisition device before the pharmaceutical agent is administered to the user, wherein the at least one biometric response and the at least one biometric parameter are used for revising the delivery parameter (e.g. dose, amount) of the pharmaceutical agent or other agent. In some embodiments, the at least one biometric parameter includes at least one of the following: blood oxygen level, body temperature, heartrate, perfusion index, blood pressure, inhalation velocity, inhalation pressure, inhalation volume, expiratory velocity, expiratory pressure, expiratory volume or exhale chemical composition. In some embodiments, methods disclosed herein can further include: determining whether the at least one biometric response for the user is within a range (e.g. parameters known for a favorable response or otherwise predictable response); and sending an alert to at least one of the user or a third-party or health professional if the at least one biometric response is not within the range (e.g. outside a predicted favorable response such as lower or higher than a desirable range).

In other aspects, methods can further include: determining, using a computing device, a response rating using the at least one biometric response. In certain embodiments, the methods can further include: providing a survey to the user; receiving at least one response to the survey; and wherein the at least one received response to the survey can be used in determining response rating. In some embodiments, methods can include: providing a test to the user; receiving at least one response to the test; and wherein the at least one received response for the test is used in determining the response rating. In other embodiments, methods can include: receiving a health record for the user; wherein the health record can be used in determining the response rating. In some embodiments, the method can further include: receiving data from at least one peripheral device, wherein the data received from the at least one peripheral device is used in determining response rating. In some embodiments, the at least one peripheral device is a pedometer.

In certain embodiments, a pharmaceutical agent can be one or more of the following: albuterol, albuterol sulfate, atropine sulfate, beclomethasone dipropionate, bitolterol mesylate, budesonide, formoterol fumarate, cromolyn sodium, desflurane, dexamethasone sodium phosphate, dornase alfa, enflurane, epinephrine, ergotamine tartrate, flunisolide, fluticasone propionate, fomoterol fumarate, halothane, iloprost, insulin, ipratropium bromide, isoetharine hydrochloride, isoflurane, isoproterenol hydrochloride, levalbuterol hydrochloride, metaproterenol sulfate, methacholine chloride, mometasone furoate, nedocromil sodium, nicotine, nitric oxide, pentamidine isethionate, pentetate calcium trisodium, pentetate zinc trisodium, pirbuterol acetate, ribavirin, salmeterol xinafoate, sevoflurane, tetrahydrocannabinol, tiotropium bromide monohydrate, tobramycin, trimcinolone acetonide, zanamivir, and combinations and derivatives thereof.

Embodiments of the present invention can also include an apparatus that includes but is not limited to: a computing device; and a pharmaceutical agent monitoring module executed by the computing device and configured to: receive, from a pharmaceutical agent delivery and biometric data acquisition device, delivery parameters of an administered pharmaceutical agent to a user; receive, from the pharmaceutical agent delivery and biometric data acquisition device, at least one biometric response of the user; and determine, using a computing device, a compliance rating using at least one of the following: the delivery parameters of the administered pharmaceutical agent and the at least one biometric response. In some embodiments, the at least one biometric response can include, but is not limited to, at least one of the following: a galvanic skin response, a blood oxygen level response, a body temperature response, a heartrate response, a perfusion index response, a blood pressure response, a retina response, an eye movement response, an inhalation velocity response, an inhalation pressure response, an inhalation volume response, an expiratory velocity response, an expiratory pressure response, an expiratory volume response or an exhale chemical composition response. In other embodiments, the at least one biometric response to the pharmaceutical agent is measured by the pharmaceutical agent delivery and biometric data acquisition device during at least one of the following intervals: less than five minutes after taking the pharmaceutical agent, less than one hour after taking the pharmaceutical agent, less than one day after taking the pharmaceutical agent, less than one week after taking the pharmaceutical agent or less than one month after taking the pharmaceutical agent.

In some embodiments, the biometric response alert module is further configured to: receive at least one biometric parameter for a user, wherein the at least one biometric parameter for the user is measured by the pharmaceutical agent delivery and biometric data acquisition device before the pharmaceutical agent is administered to the user, wherein the at least one biometric response and the at least one biometric parameter are used for revising the delivery parameter of the pharmaceutical agent. In some embodiments, the at least one biometric parameter includes at least one of the following: blood oxygen level, body temperature, heartrate, perfusion index, blood pressure, inhalation velocity, inhalation pressure, inhalation volume, expiratory velocity, expiratory pressure, expiratory volume or exhale chemical or other agent composition and/or levels of the chemical or agents in the composition.

In some embodiments, the biometric response alert module is further configured to: determine whether the at least one biometric response for the user is within a range (e.g. predetermined favorable range); and send an alert to at least one of the user or a third-party or healthcare provider if the at least one biometric response is not within the range (e.g. higher or lower than the predetermined favorable range).

In other embodiments, the biometric response alert module can be further configured to: determine a response rating using the at least one biometric response. In some embodiments, the biometric response alert module can be further configured to: provide a survey to the user; receive at least one response to the survey; and wherein the at least one received response to the survey is used in determining the response rating. In some embodiments, the biometric response alert module can be further configured to: provide a test to the user; receive at least one response to the test; and wherein the at least one received response for the test can be used in determining the response rating. In some embodiments, the biometric response alert module can be further configured to: receive a health record for the user; and wherein the health record can be used in determining the response rating.

In some embodiments, the biometric response alert module can be further configured to: receive data from at least one peripheral device; wherein the data received from the at least one peripheral device can be used in determining the response rating. In some embodiments, the at least one peripheral device can be a pedometer.

In other aspects, the pharmaceutical agent of certain embodiments disclosed herein can be one or more of the following: albuterol, albuterol sulfate, atropine sulfate, beclomethasone dipropionate, bitolterol mesylate, budesonide, formoterol fumarate, cromolyn sodium, desflurane, dexamethasone sodium phosphate, dornase alfa, enflurane, epinephrine, ergotamine tartrate, flunisolide, fluticasone propionate, fomoterol fumarate, halothane, iloprost, insulin, ipratropium bromide, isoetharine hydrochloride, isoflurane, isoproterenol hydrochloride, levalbuterol hydrochloride, metaproterenol sulfate, methacholine chloride, mometasone furoate, nedocromil sodium, nicotine, nitric oxide, pentamidine isethionate, pentetate calcium trisodium, pentetate zinc trisodium, pirbuterol acetate, ribavirin, salmeterol xinafoate, sevoflurane, tetrahydrocannabinol, tiotropium bromide monohydrate, tobramycin, trimcinolone acetonide, zanamivir, and combinations and derivatives thereof.

Embodiments of the present invention can also include, but are not limited to, a computer program product comprising a non-transitory computer readable storage medium containing program code, the computer program code when executed by a processor causes the processor to: receive, from a pharmaceutical agent delivery and biometric data acquisition device, delivery parameters of an administered pharmaceutical agent to a user; receive, from the pharmaceutical agent delivery and biometric data acquisition device, at least one biometric response of the user; and determine, using a computing device, a compliance rating using at least one of the following: the delivery parameters of the administered pharmaceutical agent and the at least one biometric response. In some embodiments, the at least one biometric response includes at least one of the following: a galvanic skin response, a blood oxygen level response, a body temperature response, a heartrate response, a perfusion index response, a blood pressure response, a retina response, an eye movement response, an inhalation velocity response, an inhalation pressure response, an inhalation volume response, an expiratory velocity response, an expiratory pressure response, an expiratory volume response or an exhale chemical composition response. In some embodiments, the at least one biometric response to the pharmaceutical agent is measured by the pharmaceutical agent delivery and biometric data acquisition device during at least one of the following intervals: less than five minutes after taking the pharmaceutical agent, less than one hour after taking the pharmaceutical agent, less than one day after taking the pharmaceutical agent, less than a week after taking the pharmaceutical agent or less than one month after taking the pharmaceutical agent.

In some embodiments, the computer program product can further include a computer product code that causes the processor to: receive at least one biometric parameter for a user, wherein the at least one biometric parameter for the user is measured by the pharmaceutical agent delivery and biometric data acquisition device before the pharmaceutical agent is administered to the user, wherein the at least one biometric response and the at least one biometric parameter are used for revising the delivery parameter of the pharmaceutical agent to the user. In accordance with these embodiments, the at least one biometric parameter can include at least one of the following: blood oxygen level, body temperature, optical imagery (e.g. retinal imagery), heartrate, perfusion index, blood pressure, inhalation velocity, inhalation pressure, inhalation volume, expiratory velocity, expiratory pressure, expiratory volume or exhale chemical composition.

In some embodiments, the computer program product can include a computer product code that causes the processor to: determine whether the at least one biometric response for the user is within a range (e.g. favorable range); and send an alert to at least one of the user or a third-party or healthcare provider if the at least one biometric response is not within the range.

In other embodiments, the computer program product can further include a computer product code that causes the processor to: determine a response rating using the at least one biometric response. In some embodiments, the computer program product further comprises computer product code that causes the processor to: provide a survey to the user; receive at least one response to the survey; and wherein the at least one received response to the survey is used in determining the response rating. In certain embodiments, the computer program product can further include a computer product code that causes the processor to: provide a test to the user; receive at least one response to the test; and wherein the at least one received response for the test is used in determining the response rating. In other embodiments, the computer program product can include a computer product code that causes the processor to: receive a health record for the user; wherein the health record can be used in determining response rating of the user. In some embodiments, the computer program product can further include a computer product code that causes the processor to: receive data from at least one peripheral device; wherein the data received from the at least one peripheral device can be used in determining the response rating. In some embodiments, the at least one peripheral device can be a pedometer.

In some embodiments, a pharmaceutical agent of methods disclosed herein can be one or more of the following: albuterol, albuterol sulfate, atropine sulfate, beclomethasone dipropionate, bitolterol mesylate, budesonide, formoterol fumarate, cromolyn sodium, desflurane, dexamethasone sodium phosphate, dornase alfa, enflurane, epinephrine, ergotamine tartrate, flunisolide, fluticasone propionate, fomoterol fumarate, halothane, iloprost, insulin, ipratropium bromide, isoetharine hydrochloride, isoflurane, isoproterenol hydrochloride, levalbuterol hydrochloride, metaproterenol sulfate, methacholine chloride, mometasone furoate, nedocromil sodium, nicotine, nitric oxide, pentamidine isethionate, pentetate calcium trisodium, pentetate zinc trisodium, pirbuterol acetate, ribavirin, salmeterol xinafoate, sevoflurane, tetrahydrocannabinol, tiotropium bromide monohydrate, tobramycin, trimcinolone acetonide, zanamivir, and combinations and derivatives thereof.

### Definitions and Terms

As used herein, the terms “subject,” “user,” and/or “patient” can include humans and other animals or mammals that are in need of treatment and capable of using or have assisted use of devices and systems as described herein. Additionally, the terms “subject,” “user,” and/or “patient” can include humans and other mammals treated in any type of environment such as a clinical setting, non-clinical setting, experimental setting, etc.

As used herein the terms “pharmaceutical,” “pharmaceutical agent,” “biological agent,” “biologic,” “monitored agent,” “agent” and “drug” can mean a pharmaceutically effective compound, and/or effective compound and/or the pharmaceutically acceptable salt of a pharmaceutically effective compound, used in the treatment of a disease or condition. For example, a pharmaceutical drug or agent contemplated herein can be used in the treatment of diseases such as asthma, bronchitis, emphysema, lung infection, cystic fibrosis, alpha-1 anti-trypsin (AAT) deficiency, chronic obstructive pulmonary disease (COPD), acute respiratory distress syndrome (ARDS), infant respiratory distress syndrome (IRDS), borderline personality disorder (BPD), and macrophage activation syndrome (MAS), among many other conditions. Useful pharmaceutical agents can be delivered via inhalation, injection, ingestion, by feeding tube, and/or sublingually, according to the present disclosure, but are not limited to only those listed in the present disclosure. Generally, the agents that can be delivered using the devices and systems of the present disclosure have been approved by the U.S. Food and Drug Administration. Other agents or drugs may be used in accordance with the devices and systems of the present disclosure; the agents listed in the present disclosure are not intended to be exhaustive.

The terms “determine,” “calculate,” and “compute,” and variations thereof, as used herein, are used interchangeably and include any type of methodology, process, mathematical operation or technique.

It is to be noted that the term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

The term “means” as used herein shall be given its broadest possible interpretation in accordance with 35 U.S.C. § 112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The term “computer-readable medium” as used herein refers to any storage and/or transmission medium that participate in providing instructions to a processor for execution. Such a medium may be commonly tangible and non-transient and can take many forms, including but not limited to, non-volatile media, volatile media, and transmission media and includes without limitation random access memory (“RAM”), read only memory (“ROM”), and the like. Non-volatile media includes, for example, NVRAM, or magnetic or optical disks. Volatile media includes dynamic memory, such as main memory. Common forms of computer-readable media include, for example, a floppy disk (including without limitation a Bernoulli cartridge, ZIP drive, and JAZ drive), a flexible disk, hard disk, magnetic tape or cassettes, or any other magnetic medium, magneto-optical medium, a digital video disk (such as CD-ROM), any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives may be considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored. Computer-readable storage medium commonly excludes transient storage media, particularly electrical, magnetic, electromagnetic, optical, magneto-optical signals.

The term “module” as used herein refers to any known or later developed hardware, software, firmware, artificial intelligence, fuzzy logic, or combination of hardware and software that is capable of performing the functionality associated with that element. Also, while the disclosure is presented in terms of exemplary embodiments, it should be appreciated that individual aspects of the disclosure can be separately claimed.

“Radio-Frequency IDentification” (RFID) refers to the use of a wireless non-contact system that uses radio-frequency electromagnetic fields to transfer data from a tag attached to an object, for the purposes of automatic identification and/or tracking. Some tags require no battery and are powered and read at short ranges via magnetic fields (electromagnetic induction) (known as passive RFID tags). Others use a local power source and emit radio waves (electromagnetic radiation at radio frequencies) (known as active RFID tags). The tag contains electronically stored information which may be read from up to several meters away. Unlike a bar code, the tag does not need to be within line of sight of the reader and may be embedded in the tracked object.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a representative block diagram of a system incorporating a pharmaceutical delivery and biometric monitoring device, according to one embodiment of the present disclosure.

FIG. 2 is a representative diagram of the top view of a pharmaceutical delivery disclosed herein and biometric monitoring device, according to one embodiment of the present disclosure.

FIG. 3 is a representative diagram of a side view of the pharmaceutical delivery and biometric monitoring device, according to one embodiment of the present disclosure.

FIG. 4 is a representative diagram of the bottom view of the pharmaceutical delivery and biometric monitoring device, according to one embodiment of the present disclosure.

FIG. 5 is a representative flow diagram of a method for authenticating a user using the pharmaceutical deliver and biometric monitoring device, according to one embodiment of the present disclosure.

FIG. 6 is a representative flow diagram illustrating a specific example of the method for authenticating a user using the pharmaceutical and biometric monitoring device described in FIG. 5.

FIG. 7 represents an exemplary flow diagram of a method 700 for monitoring delivery parameters of an administered pharmaceutical agent.

FIGS. 8A-8AA illustrate a user interface that implements the features and operations of FIG. 7, in accordance with the embodiments of this disclosure.

FIGS. 9A-9N illustrate exemplary embodiments of a third-party interface that implements the features and operations of FIG. 7, in accordance with the embodiments of this disclosure.

FIG. 10 represents an illustration of a block diagram of exemplary network operating environment for computing devices that implement the features and operations of FIGS. 7-9N.

FIG. 11 represents an illustration of a block diagram demonstrating one exemplary computing device architecture 1100 capable of implementing the features and operations of FIGS. 7-9N.

FIG. 12 represents an illustration of a block diagram of an exemplary web server architecture 1200 for implementing the features and operations of FIGS. 7-9N.

### DETAILED DESCRIPTION

Embodiments of the present disclosure generally relate to devices and systems for administering pharmaceutical and biological agents. More specifically, the present disclosure provides devices, methods and systems for biometric data acquisition and monitoring before, during, and after administration of pharmaceutical and/or biological agents to a subject.

Embodiments of the devices of the present disclosure can include three principal components: a scanner to verify and/or authenticate a user (e.g., a fingerprint scanner), a biometric sensor to acquire user biometric data (e.g., a pulse oximeter), and a pharmaceutical delivery component (e.g., an inhalation canister) to deliver a pharmaceutical, biological or other monitored agent to the user. In accordance with these embodiments, the devices of the present disclosure can be handheld, allowing an authenticated user or caregiver to deliver a pharmaceutical or biological agent or other monitored agent while acquiring user biometric data before, during and/or after administration of the pharmaceutical or biological agent. In some embodiments, the devices of the present disclosure can also facilitate transfer of user biometric data to an authorized caregiver, health professional or physician, which can be used by the caregiver, healthcare provider or physician to evaluate accurately the user's condition and provide more effective treatment options.

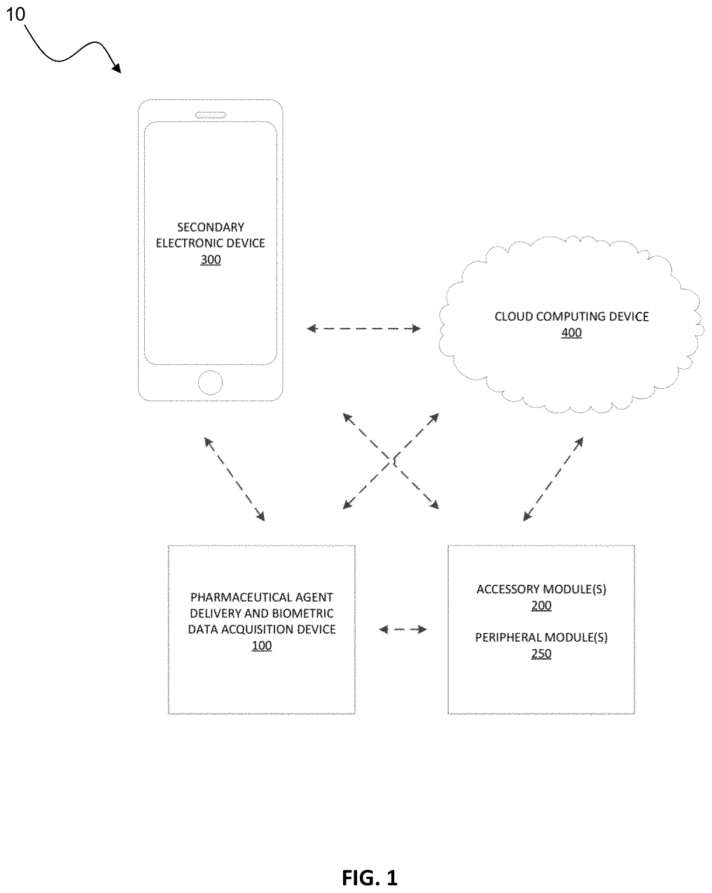

FIG. 1 is a representative block diagram of a system 10 incorporating the pharmaceutical delivery and biometric data acquisition device 100, according to one embodiment of the present disclosure. The system 10 includes a pharmaceutical agent or other agent delivery and biometric data acquisition device 100, one or more accessory modules 200, one or more peripheral modules 250, a secondary electronic device 300, and a cloud computing device 400 all of which can be communicatively coupled, using either a wired or wireless connection. However, in some embodiments, the devices 100, 200, 250, 300, 400 illustrated in FIG. 1 do not always have to be connected to one another and may only establish a connection intermittently. Furthermore, in some embodiments, the pharmaceutical agent delivery and biometric data acquisition device 100 may not connect to all the other devices 200, 250, 300, 400 illustrated in FIG. 1, but may only connect to one of the other devices 200, 250, 300, 400. For example, in some embodiments, the pharmaceutical agent delivery and biometric data acquisition device 100 may only connect to the secondary electronic device 300. In these embodiments, the secondary electronic device 300 can then connect to the cloud computing device 400. However, this is only an example and not meant to be limiting.

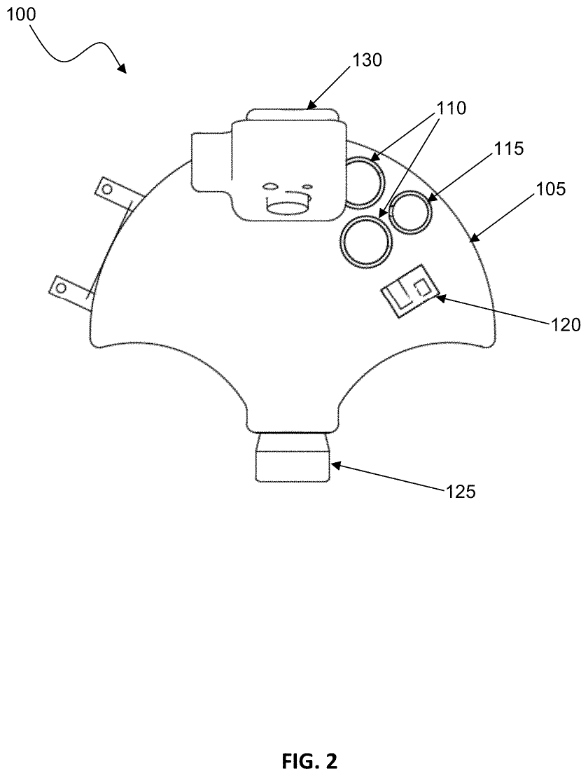

The pharmaceutical agent delivery (or other agent) and biometric data acquisition device 100, the accessory module(s) 200 and the peripheral modules(s) 250 are discussed in more detail below in FIGS. 2-4. In the illustrated example, the secondary electronic device 300 can be a smartphone. However, other exemplary secondary electronic device(s) 300 can include, but are not limited to, a telephone, a laptop computer, a tablet computer, a personal digital assistant (PDA), a digital camera or other image recording device, a gaming device, a desktop computer, a fitness tracking device, a digital display device, a docking station, or a security terminal or station. The cloud computing device 400 may be implemented, for example, as one or more servers which may be communicatively coupled to the Internet, and which may be co-located or geographically distributed.

As illustrated in FIG. 2, the pharmaceutical agent delivery and biometric data acquisition devices 100 of the present disclosure include a housing unit 105, which may be configured to contain a battery, a real time clock, and a processing device for operating a plurality of biometric sensors. The structure of the housing unit 105 may be generally configured to enable a user to grasp and operate the device without interfering with biometric data acquisition before, during, or after a pharmaceutical agent is being delivered. For example, certain devices contemplated herein can have wing-like projections facilitating grasp of the device by the user, as illustrated in FIG. 2. Other similar shapes and configurations can readily be ascertained by one of ordinary skill in the art based on the present disclosure and what is known in the art.

In some embodiments, the wing-like projections of the housing unit 105 can provide sufficient structure or surface area permitting the user to interface with various biosensors that can be included in or on the surface of the device. For example, the device can include one or more galvanic skin response sensors 110 located on the top portions of either or both of the wing-like projections of the housing unit 105 (FIG. 2). The galvanic skin response (GSR) sensors 110 of the present disclosure, can also be referred to as electrodermal response (EDR) sensors, psychogalvanic reflex (PGR) sensors, skin conductance response (SCR) sensors, or skin conductance level (SCL) sensors, generally measure electrical conductance of the skin, which can vary depending, for example, on the state of sweat or other condition of the skin. Sweating is generally considered to be controlled by the sympathetic nervous system; therefore, electrical skin conductance can provide psychological and/or physiologic biometric data about the user. In general, if the sympathetic branch of the autonomic nervous system is highly aroused, then sweat gland activity also increases, which in turn increases skin conductance. In this way, skin conductance can be used as a biometric measurement of emotional and sympathetic responses, which can be used to evaluate, for example, the efficacy and/or side effects caused by various pharmaceutical agents before, during, or after delivery of the pharmaceutical agents.

In other embodiments, the housing unit 105 can provide sufficient structure for incorporating one or more temperature sensors (FIG. 2). For example, the device can include one or more fingertip temperature sensors 115 positioned on the top portions of either of the wing-like projections of the housing unit 105 such that the temperature of a user's skin can be acquired and/or monitored before, during, and/or after a pharmaceutical agent or other agent is being delivered and/or administered. Typically, the temperature at the surface of a subject's skin changes according to blood circulation through the body tissue. The small blood vessels crossing through the tissue are surrounded by fibers of smooth muscle, which are controlled by the sympathetic nervous system. In a state of increased exertion, excitement and stress, these muscle fibers contract, causing a stenosis of vasculature. This leads to a reduction of skin temperature, because blood circulation through the tissue is reduced. In contrast, in a state of relaxation, the musculature is also bound to relax, causing the vasculature to expand. Hence, the skin temperature rises. Mental stress can lead to a lower peripheral perfusion and a decrease of skin temperature at the hands, caused by increased activity of the sympathetic nervous system. In this way, temperature of the skin at a user's fingertip can be used to as a biometric measurement for evaluating, for example, the efficacy and/or side effects caused by various pharmaceutical agents before, during, and/or after delivery of the pharmaceutical agents or other agents.

In some embodiments, the housing unit 105 can provide sufficient structure for incorporating one or more ambient temperature sensors for measuring the air temperature immediately surrounding the device, thus reflecting changes in the environmental conditions. In some embodiments, the ambient temperature sensor can be integrated with the galvanic skin response sensors and/or the fingertip temperature sensors in order to account for alterations in the environmental conditions. The integration of the various temperature sensors can provide more accurate temperature measurements of the subject for evaluating, for example, the efficacy and/or side effects caused by various pharmaceutical agents before, during, and/or after delivery of the pharmaceutical agents.

In some embodiments, a fingertip sensor can be included in the devices of the present disclosure to measure a user's heart rate. For example, a fingertip heart rate sensor unit can include an infrared light-emitting-diode (IR LED) and a photo diode, such that a user's fingertip can be placed over the sensor unit. The IR LED can transmit an infrared light into the fingertip, a part of which may be reflected back from the blood inside the finger arteries. The photo diode then senses the portion of the light that is reflected back. The intensity of reflected light depends upon the blood volume inside the fingertip, which varies every time the heart beats in accordance with changes in the amount of reflected infrared light detected by the photo diode. Other similar methods of detecting heart rate using a fingertip sensor can readily be ascertained by one of ordinary skill in the art based on the present disclosure. Monitoring a user's heart rate can be an important biometric measurement for evaluating, for example, the efficacy and/or side effects caused by various pharmaceutical agents before, during, or after delivery of the pharmaceutical agents. Other methods for measuring/detecting heart rate are known in the art and can be adapted to the device as needed.

In some embodiments, the housing unit 105 can provide sufficient structure for incorporating one or more pulse oximeters 120 (FIG. 2). For example, a pulse oximeter 120 can be used to measure the oxygen level (or oxygen saturation) in a subject's blood. Typically, the pulse oximeter 120 can be placed on a thin part of a subject's body, usually a fingertip, and two wavelengths of light are passed through the fingertip to a photodetector. The photodetector measures the changing absorbance at each of the wavelengths, allowing it to determine the absorbance due to the pulsing arterial blood alone. The pulse oximeter 120 can be used to assess a user's blood oxygenation levels and determining the effectiveness of, or need for, supplemental oxygen. The pulse oximeter 120 can also be used as a biometric measurement for evaluating, for example, the efficacy and/or side effects caused by various pharmaceutical agents before, during, and/or after delivery of the pharmaceutical agents. The pulse oximeter 120 can also be used to determine noninvasively a subject's hemoglobin level within 1-2 minutes without requiring any further equipment.

In some embodiments, the housing unit 105 can be coupled to a mouthpiece 125 that facilitates inhalation and exhalation of air from a user to the device (FIG. 2). In some embodiments, the mouthpiece 125 and the device 100 can be used for the treatment of asthma and/or asthmatic conditions, wherein for example, clenbuterol is delivered to a subject and various pulmonary biometrics are evaluated before, during, and/or after delivery of the clenbuterol in order to assess the subject's response to the clenbuterol.

In some embodiments, the mouthpiece 125 can be functionally coupled to a pulmonary function adaptor. The pulmonary function adaptor can be generally cylindrical in shape for insertion into a horizontal port in the device 100. In certain embodiments, the pulmonary function adaptor can facilitate the measurement the velocity, depth and composition of a subject's breath, measurements which are important biometrics for evaluating the health of the subject. For example, a device of the present disclosure having a pulmonary function adaptor can include one or more air pressure sensors which measure air pressure, often stated in terms of force per unit area. A pressure sensor typically acts as a transducer by generating an electrical or digital signal as a function of the pressure imposed. Sensors can be used to measure variables such as air flow, speed, and altitude. Air pressure sensors can alternatively be called pressure transducers, pressure transmitters, pressure senders, pressure indicators, piezometers and manometers, among other names, as would be appreciated by one of ordinary skill in the art based on the present disclosure and knowledge in the art.

Suitable materials that can be used to construct the housing unit 105, the mouthpiece 125, and/or the pulmonary function adaptor include, but are not limited to, various plastics and polymers materials, such as polystyrene (PS), polycarbonate (PC), acrylonitrile-butadiene-styrene (ABS), polybutylene terephthalate (PBTP), styrene acrylonitrile (SAN), polyamide (PA), polyoxymethylene (POM), polyphenylene oxide (PPO), PE, PP, PTFE and homopolymers and copolymers of these plastics and similar materials known in the art. The plastics may also be used in a filled or fiber-reinforced form, and/or coupled to portions of metals or metal alloys, such as aluminum, titanium, steel, and combinations thereof. The materials used to construct the housing unit 105 and/or the mouthpiece 125 can be surface-coated, for example with paints, varnishes or lacquers. The use of color plastics, for example colored with pigments, is also possible. In some embodiments, the housing unit 105, the mouthpiece 125, and/or the pulmonary function adaptor can be coated with substances that help to prevent contamination from microorganisms, bacteria, fungi, viruses, and the like. The coatings can be active pharmaceutical agents that reduce the growth and/or survival of these harmful microorganisms (e.g., anti-bacterial substances), or the coatings can function passively to prevent contamination, for example, by preventing adherence of these microorganism to the housing unit 105, the mouthpiece 125, and/or the pulmonary function (e.g., wetting agents).

In some embodiments, air pressure sensors can be coupled with one or more sensors designed to assess the chemical and/or gaseous composition of a user's breath. For example, the device of the present disclosure can include one or more sensors to detect carbon dioxide levels expired and/or produced by a user. A carbon dioxide sensor or CO₂ sensor typically includes infrared gas sensors (e.g., NDIR sensors) and chemical gas sensors, which can help assess the function of a subject's lungs. NDIR sensors are typically spectroscopic sensors used to detect CO₂ by its characteristic absorption. The key components include an infrared source, an interference (wavelength) filter, and an infrared detector. In some embodiments, a user breathes air through the mouthpiece 125, and the sensor measures the absorption of the characteristic wavelength of light. CO₂ sensors can also be functionally coupled with one or more air pressure sensors described above to capture a user's biometric data pertaining to both CO₂ levels and respiration rate, key biometrics used to evaluate a subject's health and disease state. Air pressure sensors and CO₂ sensors can also be used to assess, for example, the efficacy and/or side effects caused by various pharmaceutical agents before, during, or after delivery of the pharmaceutical agents. In other embodiments, sensors can be used to detect odors in a subject's breath, including an ammonia-like odor, which can be indicative of kidney failure, and/or a fruity odor, which can be indicative of ketoacidosis/diabetes and/or anorexia and other disorders.

Other sensors can also be included in the devices of the present disclosure, as would be readily appreciated by one of ordinary skill in the art based on the present disclosure and knowledge to one of skill in the art. For example, the devices of the present disclosure can include global positioning system (GPS) sensors, chemical sensors, thermal sensors, magnetic sensors, radiation sensors, proximity sensors, acoustic sensors, vibration sensors, acceleration sensors, moisture sensors, and the like. In some embodiments, the device can be equipped with a sensor or monitor capable of measuring a subject's blood glucose levels, as well as determining if the subject's blood glucose levels are within a certain range (e.g. normal or outside the range of normal).

In other embodiments, a thermal imaging sensor can be included in the devices of the present disclosure to facilitate user authentication and/or as a biometric sensor. A thermal imaging sensor can be integrated with the image acquisition device to facilitate the scanning and processing of a thermal image of one or more portions of a subject's face and/or the subject's entire body. In some embodiments, the thermal imaging sensor can be used to evaluate whether the subject has a medical condition (e.g., fever) that may require immediate attention. In such embodiments, the device can be configured to send an alert message to the subject to seek immediate medical attention.

In some embodiments, a user (e.g., authenticated user, healthcare provider or associate of the authenticated user) can set one or more alarms using the device, such as one or more medication alarms, which can present a stimulus to the user with or without an accompanying text-based message to, for example, take one or more doses of one or more pharmaceutical or biological agents. The alarm can be a visual (e.g., flashing light) and/or auditory (e.g., ringing bell sound) stimulus that can be emitted from the device. The alarm can also be pushed to another device, such as a mobile phone or computing device. In some embodiments, the alarm can take the form of an email, text message, a message from a third party mobile phone application and the like. Similarly, a user can set one or more biometric alarms, which can present a similar stimulus to the user to, for example, obtain and record one or more biometrics using the device.

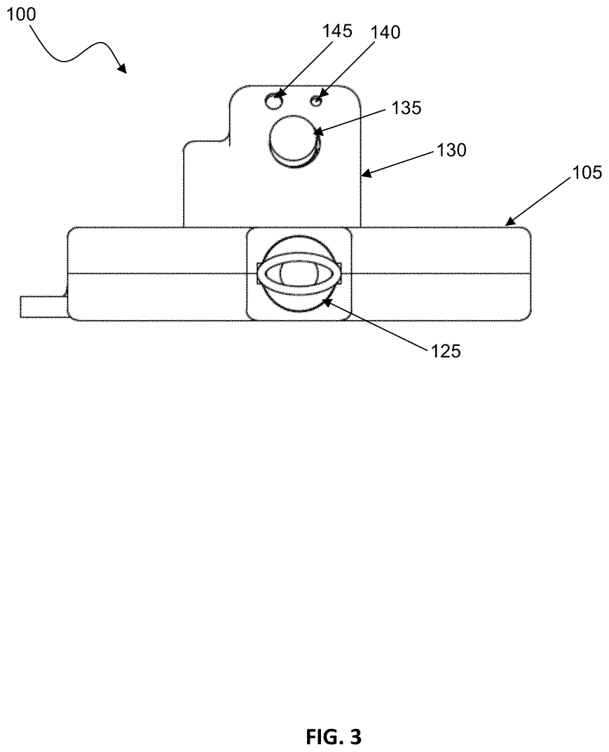

In some embodiments, the device of the present disclosure includes an image acquisition device 130 (FIG. 3). The image acquisition device 130 may be generally positioned on the device such that it is centrally aligned with the mouthpiece 125. The image acquisition device 130 comprises a lens 135, an image sensor, and signal wires which operatively connect the image acquisition device 130 to the processor in the device. In some embodiments, the image acquisition device may be a digital camera. The image acquisition device 130 can be mounted on the housing unit 105 and be electrically coupled to the processor of the device. The image acquisition device 130 generally faces the same direction as that of the mouthpiece 125, such that when a user's mouth engages the mouthpiece 125, the user's eyes will be facing the lens of the image acquisition device 130.

In one manner of operation, the image acquisition device 130 can capture a digital image and/or a series of digital images (e.g., a digital video) before, during, or after delivery of a pharmaceutical or other monitored agent. In some embodiments, the image sensor can detect a user's pupils and capture one or more images of the user's pupils before, during, and/or after delivery of a pharmaceutical agent in order to assess the efficacy and/or side effects caused by the pharmaceutical or other monitored agent. In other embodiments, the image acquisition device 130 can be used to assess the color of a user's eye, including but not limited to, the color of a user's sclera. For example, certain conditions can cause a subject's eyes to appear yellow, which can indicate dysfunction in one or more bodily organs such as the liver, gallbladder, or pancreas. Yellowing of the sclera can be used to diagnose various conditions, including alcohol abuse, hepatitis (A, B, C, D, and E), liver cancer, liver infection, and non-alcoholic fatty liver disease. In other embodiments, an image acquisition device 130 can be used to assess pupil dilation or severe reddening of an eye known to be linked to side effects of certain agents.

In other embodiments, the image acquisition device 130 can be configured to capture a digital image and/or a series of digital images that can be transferred to an auxiliary electronic device and viewed by a caregiver or health provider for diagnostic purposes. For example, the pharmaceutical agent delivery and biometric data acquisition device 100 can have an activation button functionally coupled to the image acquisition device 130 to enable a user to engage the activation button and capture a digital image or video of, for example, information pertaining to the pharmaceutical or other monitored agent (e.g., dose, lot number, etc.) or a physical manifestation of a disease condition located on the subject (e.g., wound, laceration, rash, allergic reaction, insect bite, swollen glands, etc).

In some embodiments, the image acquisition device 130 can be configured to take a picture of a subject's retina to evaluate the vascularization of the retina and/or whether the subject has a retinal vascular occlusion. A retinal vascular occlusion occurs when one of the veins or arteries carrying blood to or from the retina becomes blocked or contains a blood clot. The blockage could occur in the main vein or main artery. Blockages could also occur in the branch of veins and arteries throughout the retina. A blockage in the vein or artery of the retina can cause blood or other fluids to build up and inhibit the retina's ability to filter light properly. When light is blocked or fluids are present, sudden loss of vision can occur. The presence of a retinal vascular occlusion or blockage can be a predictor of an increased likelihood that the subject will experience a stroke or other life-threatening condition.

The pharmaceutical agent delivery and biometric data acquisition device 100 can also be equipped with a microphone that may or may not be functionally coupled to the image acquisition device 130 to facilitate real-time and/or recorded audio and/or video communication with a caregiver for diagnostic purposes.

The image acquisition device 130 can also include one or more visual indicators operatively coupled to the image acquisition device and facing the same direction as the lens 135, which emit at least one light signal. In some embodiments, the visual indicator can be an LED that emits green light 140. In other embodiments, the visual indicator can be an LED that emits white light 145. These and other visual indicators can be used to communicate directions to the user, such as when to administer a pharmaceutical agent (e.g., inhale or ingest a pharmaceutical agent). These and other visual indicators can also be used to facilitate the acquisition of biometric data from the user, such as emitting a flash of light to dilate a user's pupils. Changes in a user's pupil size or pupil dilation can be an important biometric measurement indicating, for example, the efficacy and/or side effects caused by the administration of a pharmaceutical or other monitored agent or caused by dose level of an administered agent. In accordance with these embodiments, negative visual indicators can then be used to adjust, change or eliminate use of the agent for the user. Additionally, the device can be configured to send instructions to a user to activate an eye tracking program that uses visual stimulation, such as pulses of light, to assess various neurological problems, including brain diseases and brain injuries (e.g., concussions). Eye tracking technology and testing protocols are well established and can obtain hundreds of data points during, for example, a 30-second test facilitated by the video recording capability of the image acquisition device 130.

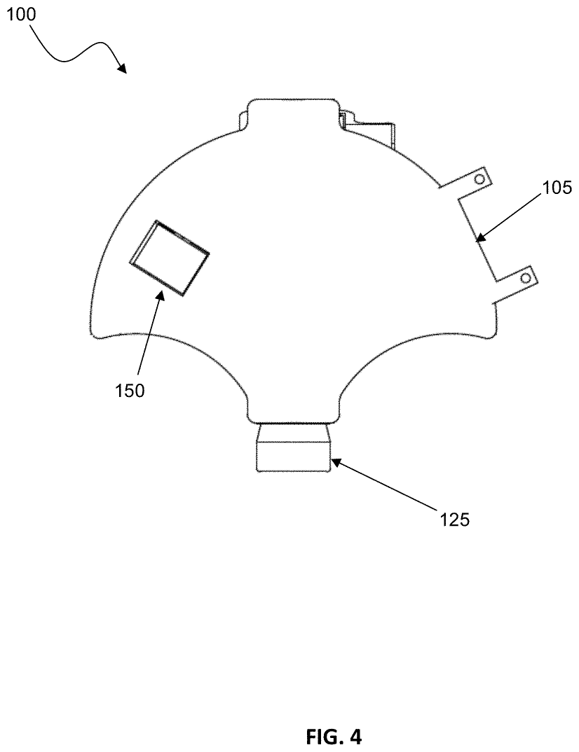

Some embodiments disclosed herein can include one or more scanners associated with the device which authenticate and/or verify the identity of a user or caregiver that will be administering a pharmaceutical or other monitored agent to a subject. In some embodiments, images captured using the image acquisition device 130 can be used for retinal scanning and/or facial recognition to prevent unauthorized users from being able to take a pharmaceutical agent meant for the imprinted user and/or tampering with the device. In other embodiments, the device of the present disclosure can include a fingerprint scanner 150 to prevent unauthorized users from administering a pharmaceutical agent and/or tampering with the device (FIG. 4). The device of the present disclosure can store in its memory a plurality of distinct user fingerprints, (e.g., biometric identifiers), and the device can be programmed to correlate a particular fingerprint with certain device settings for a particular user. In this way, the device of the present disclosure can be used by more than one user, if desired, for example, a family of users, without the need for multiple devices for each person in need thereof or for each pharmaceutical or monitored agent being administered. In other aspects, the device can be configured to be accessed specifically by an authorized user such as a nurse, health provider, parent or other caregiver, and the nurse, health provider, parent or caregiver's fingerprint or other biometric identifier can be used to access the patient's settings on the device, as the patient may need to be restricted from using the device on his/her own or the patient may not be capable of using the device without supervision or aide (e.g., a child or elderly person). The fingerprint scanner 150 can also be used in conjunction with a lockout mechanism in which the device will be “locked out” or inactive for a given operation of a particular delivery program if the user's fingerprint is not recognized.

Aspects of the device of the present disclosure can include memory in electrical communication with the processor of the device and configured to facilitate the acquisition and storage of biometric data acquired using various biometric sensors from one or more users. Biometric data can include, but is not limited to, images, air flow rates, air composition, fingerprints, oxygen levels, carbon dioxide levels, skin electrical conductance measurements, time, temperature, heat, user identification, dosages, usage rates, medication batch numbers, bar codes, and any other biometric data that can be captured using the various biometric sensors of the present disclosure. User biometric data can be stored and uploaded/downloaded wirelessly to a variety of memory storage and data processing devices, including but not limited to, cell phones, smart phones, watches, computers, laptops, tablets, servers, and the like. User biometric data can also be stored and uploaded/downloaded via a wire or cable to a variety of other memory storage and data processing devices, including but not limited to, cell phones, smart phones, watches, computers, laptops, tablets, servers, and the like. In such embodiments, the device can have one or more data transfer ports. The ability to acquire and store a subject's biometric data over time provides physicians with more accurate diagnostic and biometric data with which to evaluate the subject, and allows for more general patient trends to be analyzed with relation to, for example, a specific disease indication.

Other aspects of the device of the present disclosure can include one or more accessory module interfaces that facilitate the functional coupling of one or more accessory modules 200 to the device. Examples of accessory modules 200 include, but are not limited to, an injectable syringe, an injectable needle, an inhaler, an inhaler canister, a syrup dispenser, a pill dispenser, a spray device, a nebulizer, a vaporizer, a misting device, an inhalation mask, and the like. The accessory module interfaces allow for one or more accessory modules 200 to be coupled to the device such that one or more pharmaceutical agents can be administered to a user.

In some embodiments, the device includes an accessory module 200 that acts as a storage container for various pharmaceutical and biological agents in various physical forms (e.g., mists, sprays, liquids, solid dosage forms, syrups and the like). The storage container can be generally cylindrically shaped so that it can be inserted into a centrally aligned port in the device, roughly aligned with the mouthpiece 125, for example. In one manner of use, the storage container may be inserted into the device, and the device records the presence of the storage container. The device can be equipped with sensors to not only detect the presence or absence of the storage container, but also the weight of the storage container. When a user manually engages with an interface on the device to eject the storage container, the device can record the time that the storage container was ejected. The user can then open or in some way manually access the contents of the storage container in order to administer one or more pharmaceutical or biological agents. For example, the user can remove from the storage container an eye dropper and administer a specific number of drops to his or her eyes. After administering the one or more pharmaceutical or biological agents, the user can reinsert the storage container in the device and this time can be recorded as well (e.g., to determine whether the user is complying with a predetermined treatment plan). The weight sensors in the device can then record whether the weight of the container has changed, and if so, this can be an indication as to whether a predetermined dose of a pharmaceutical or biological agent was administered.

For example, accessory 200 module can include a vaporizing element positioned within the housing unit 105 itself, or coupled to the housing unit 105 via an accessory module interface. The vaporizing element can be electrically coupled to the processor of the device, such that the user can activate the vaporizing unit in conjunction with the activation of one or more biosensors to facilitate the acquisition of biometric data using the biosensors before, during, and/or after administration of a pharmaceutical agent using the vaporizing element. The vaporizing element can include a heating element typically capable of producing temperatures, for example, between 300° F. and 500° F. Alternatively, the vaporizing element can include an ultrasonic element emitting ultrasonic frequencies that heats and/or cavitates and vaporizes medication within the housing unit 105. When, for example, a pharmaceutical agent in fluid form is brought in contact or adjacent to the vaporizing element, the fluid becomes vaporized, and the fluid vapors can be inhaled by a user.

In some embodiments, the pharmaceutical or other monitored agent can be contained within a sealed container having a tamper resistant construction (e.g., a canister or inhaler used to deliver clenbuterol). In other embodiments, the pharmaceutical agent can be contained within a sterile syringe dispensing device or mister (e.g., insulin). The accessory module interfaces of the present disclosure will be configured to allow a variety of such modules to be coupled to the device, such that the device can facilitate the administration of the pharmaceutical or other monitored agent to a user. For example, the device can include an actuator mounted on the housing unit 105 and electrically coupled to the processor of the device. The actuator can be configured to, for example, activate a vaporizing element to vaporize a pharmaceutical agent. The actuator can also be coupled to a biosensor, such as a fingerprint scanner, to allow vaporization of a pharmaceutical agent only when the fingerprint of an authorized user is detected.

In certain aspects, the device can include a mechanism for monitoring amount or dosage of a pharmaceutical or other monitored agent administered to a subject or user. For example, the device can include an IR transmitter and receiver which can be used to evaluate the distance a syringe dispenser has travelled in relation to a starting point, which can correspond to a single dose of a pharmaceutical agent. Additionally, the device can include a radio-frequency identification (RFID) reader, which can be used to assess the batch, date, amount and source of a particular pharmaceutical or other monitored agent.

In some embodiments, peripheral accessory modules or peripheral modules can be functionally coupled to the device of the present disclosure via peripheral module interfaces rather than accessory module interfaces. In certain devices, a peripheral module requires its own power source separate from the device, which can preclude the peripheral module from being coupled to the device via an accessory module interface. The peripheral accessory interface can be a port, including any electronic data transfer port, such as a USB port, a firewire port, and the like. Peripheral modules can include, for example, blood pressure monitors, blood glucose monitors, CPAP machines, and/or electrocardiogram machines, as well as peripheral modules for providing additional power to the device, such as a battery or a battery charging device, and devices that enable the use of Bluetooth™ and Wi-Fi™ compatibility. As with accessory modules, some peripheral modules can be functionally coupled to the processor of the device of the present disclosure to facilitate the delivery of a pharmaceutical or other monitored agent and/or the acquisition of biometric data from a user.

In some embodiments, a peripheral module can be a secondary electronic device, such as a docking station. The docking station can be used to charge the device, and can include various other accessory ports, such as an Ethernet port and/or a communication port to support a telephone landline. Additionally, the docking station can be configured to sterilize the device between uses and/or between uses by multiple users to minimize and/or prevent bacterial, fungal, and viral contamination. For example, the docking station can be configured to contain one or more sources of UV light to reduce contamination when the device is housed in the docking station. The docking station can also be configured to combine the sterilization power of UV light, purifying hydroxyl and/or activated oxygen radicals, and photo-ionization to purify the internal and external components of the device. To facilitate this sterilization process, the docking station can be equipped with various air flow mechanisms, which assist with both the activation and circulation of the hydroxyl and oxygen radicals through the device. These and other sterilization mechanisms can be included in the docking station, as would be readily recognized by one of ordinary skill in the art based on the present disclosure.

In other embodiments, the device can be coupled to a CPAP machine (Continuous Positive Airway Pressure) or a baby monitor (e.g., monitors used to assess Sudden Infant Death Syndrome, or SIDS), or other such medical monitoring peripheral devices. The device can be used to acquire further biometric data that is not possible using the medical monitoring peripheral device, and/or the device can be used to integrate the biometric data acquired using the medical monitoring peripheral device. In other embodiments, the device can be coupled to a motor vehicle, such that operation of the motor vehicle (e.g., starting a car) by the subject will only be allowed if certain biometric parameters are met. This feature can help prevent a subject who is taking various pharmaceutical and biological agents from operating a motor vehicle while impaired.

Various pharmaceutical and biological agents can be administered using the devices, systems, and methods of the present disclosure. These pharmaceutical, biological and other monitored agents can include, but are not limited to, those approved by the U.S. Food and Drug Administration, such as, for example, albuterol, albuterol sulfate, atropine sulfate, beclomethasone dipropionate, bitolterol mesylate, budesonide, formoterol fumarate, cromolyn sodium, desflurane, dexamethasone sodium phosphate, dornase alfa, enflurane, epinephrine, ergotamine tartrate, flunisolide, fluticasone propionate, fomoterol fumarate, halothane, iloprost, insulin, ipratropium bromide, isoetharine hydrochloride, isoflurane, isoproterenol hydrochloride, levalbuterol hydrochloride, metaproterenol sulfate, methacholine chloride, mometasone furoate, nedocromil sodium, nicotine, nitric oxide, pentamidine isethionate, pentetate calcium trisodium, pentetate zinc trisodium, pirbuterol acetate, ribavirin, salmeterol xinafoate, sevoflurane, tetrahydrocannabinol, tiotropium bromide monohydrate, tobramycin, trimcinolone acetonide, zanamivir, and combinations and derivatives thereof.

Pharmaceutical, biological or other agents that can be administered using the devices, systems, and methods of the present disclosure include, but are not limited to, those agents that have not yet been approved by the U.S. Food and Drug Administration but are known to be of use to treat a disease or a condition, such as, for example, 13-cis-retinoic acid, 2-pentenylpenicillin, L-alphaacetylmethadol, S-adenosylmethionine, acebutolol, aceclofenac, acetaminophen, acetaphenazine, acetophenazine, ademetionine, adinazolam, adrafinil, ahnotriptan, albuterol, albuterol, albuterol sulfate, alfentanil, alfentanil HCl, alizapride, allylprodine, alminoprofen, almotriptan, alperopride, alphaprodine, alpidem, alseroxion, amantadine, ambrisentan, amesergide, amfenac, aminopropylon, amiodarone HCl, amisulpride, amitriptyline, amixetrine, amlodipine, amoxapine, amoxicillin, amperozide, amphenidone, amphetamine, ampicillin, amylpenicillin, andropinirole, anileridine, apazone, apomorphine, apomorphinediacetate, atenolol, atropine sulfate, azacyclonol, azasetron, azatadine, azidocillin, bacille Calmette-Guerin, baclofen, beclomethasone dipropionate, benactyzine, benmoxine, benoxaprofen, benperidol, benserazide, benzpiperylon, benzquinamide, benztropine, benzydramine, benzylmorphine, benzylpenicillin, bezitramide, binedaline, biperiden, bitolterol, bitolterol mesylate, brofaromine, bromfenac, bromisovalum, bromocriptine, bromopride, bromperidol, brompheniramine, brucine, buclizine, budesonide, budesonide; formoterol fumarate, budipine, bufexamac, buprenorphine, bupropion, buramate, buspirone, butaclamol, butaperazine, butorphanol, butriptyline, cabergoline, caffeine, calcium-N-carboamoylaspartate, cannabinoids, Cannabis, Cannabis oil, captodiamine, capuride, carbamazepine, carbcloral, carbenicillin, carbidopa, carbiphene, carbromal, carfecillin, carindacillin, caroxazone, carphenazine, carpipramine, carprofen, cefazolin, cefinetazole, cefinetazole, cefoxitin, cephacetrile, cephalexin, cephaloglycin, cephaloridine, cephalosporin C, cephalosporins, cephalotin, cephamycin A, cephamycin B, cephamycin C, cephamycins, cepharin, cephradine, cericlamine, cetrizine, chloralbetaine, chlordiazepoxide, chlorobutinpenicillin, chlorpheniramine, chlorpromazine, chlorprothixene, choline, cialis, cilazaprol, cilostazol, cinchophen, cinmetacin, cinnarizine, cipramadol, citalopram, clebopride, clemastine, clobenzepam, clocapramine, clomacran, clometacin, clometocillin, clomipramine, clonidine, clonitazene, clonixin, clopenthixol, clopriac, clospirazine, clothiapine, clovoxamine, cloxacillin, clozapine, codeine, cotinine, cromolyn sodium, cyamemazine, cyclacillin, cyclizine, cyclobenzaprine, cyclosporin A, cyproheptadine, deprenyl, desflurane, desipramine, dexamethasone sodium phosphate, dexfenfluramine, dexmedetomidine, dextroamphetamine, dextromoramide, dextropropoxyphene, diamorphine, diazepam, diclofenac, dicloxacillin, dihydrocodeine, dihydroergokryptine, dihydroergotamine, diltiazem, diphenhydramine, diphenicillin, diphenidol, diphenoxylate, dipipanone, disulfiram, dolasetronmethanesulfonate, domeridone, dornase alfa, dosulepin, doxepin, doxorubicin, doxylamine, dronabinol, droperidol, droprenilamin HCl, duloxetine, eletriptan, eliprodil, enalapril, enciprazine, enflurane, entacapone, entonox, ephedrine, epinephrine, eptastigmine, ergolinepramipexole, ergotamine, ergotamine tartrate, etamiphyllin, etaqualone, ethambutol, ethoheptazine, etodolac, famotidine, fenfluramine, fentanyl, fexofenadine, fientanyl, flesinoxan, fluconazole, flunisolide, fluoxetine, flupenthixol, fluphenazine, flupirtine, flurazepam, fluspirilene, fluticasone propionate, fluvoxamine, formoterol fumarate, frovatriptan, gabapentin, galanthamine, gepirone, ghrelin, glutathione, granisetron, haloperidol, halothane, heliox, heptylpenicillin, hetacillin, hydromorphone, hydroxyzine, hyoscine, ibuprofen, idazoxan, iloprost, imipramine, indoprofen, insulin (recombinant human), ipratropium bromide, iproniazid, ipsapiraone, isocarboxazid, isoetharine hydrochloride, isoflurane, isometheptene, isoniazid, rifampin, pyrazinamide, ethambutol, isoproterenol, isoproterenol hydrochloride, isoproterenol bitartrate, isosorbide dinitrate, ketamine, ketoprofen, ketorolac, ketotifen, kitanserin, lazabemide, leptin, lesopitron, levalbuterol hydrochloride, levodopa, levorphanol, lidocaine, lisinopril, lisuride, lofentanil, lofepramine, lomustine, loprazolam, loratidine, lorazepam, lorezepam, loxapine, maprotoline, mazindol, mazipredone, meclofenamate, mecloqualone, medetomidine, medifoxamine, melperone, memantine, menthol, meperidine, meperidine HCl, meptazinol, mesoridazine, metampicillin, metaproterenol, metaproterenol sulfate, methacholine chloride, methadone, methaqualone, methicillin, methprylon, methsuximide, methyphenidate, methyprylon, methysergide, metoclopramide, metofenazate, metomidate, metopimazine, metopon, metoprolol, metralindole, mianserin, midazolam, milnacipran, minaprine, mirtazapine, moclobemide, mofegiline, molindrone, mometasone furoate, morphine, nabilone, nadolol, nafcillin, nalbuphine, nalmefene, nalorphine, naloxone, naltrexone, naratriptan, nedocromil, sodium, nefazodone, nefopam, nicergoline, nicotine, nicotine, nifedipine, nisoxetine, nitrous oxide, nitroglycerin, nomifensine, nortriptyline, obestatin, olanzapine, omoconazole, ondansetron, orphenadrine, oxprenolol, oxycodone, palonosetron, papaveretum, papaverine, paroxetine, pemoline, penfluridol, penicillin N, penicillin O, penicillin S, penicillin V, pentamidine isethionate, pentazocine, pentetate, calcium trisodium, pentetate, zinc trisodium, pentobarbital, peptides, pergolike, pericyazine, perphenazine, pethidine, phenazocine, pheneizine, phenobarbital, phentermine, phentolamine, phenyhydrazine, phosphodiesterase-5, pilocarpine, pimozide, pipamerone, piperacetazine, pipotiazine, pirbuterol acetate, pirbuterolnaloxone, piroxicam, pirprofen, pizotifen, pizotyline, polyeptides, polypeptide YY, pramipexole, prentoxapylline, procaine, procaterol HCl, prochlorperazine, procyclidine, promazine, promethazine, propacetamol, propanolol, propentofylline, propofol, propoxyphene, propranolol, proteins, protriptyline, quetiapine, quinine, rasagiline, reboxetine, remacemide, remifentanil, remoxipride, retinol, ribavirin, rimonabant, risperidone, ritanserin, ritodrine, rizatriptan, roxindole, salicylate, salmeterol xinafoate, salmetrol, scopolamine, selegiline, sertindole, sertraline, sevoflurane, sibutramine, sildenafil, spheramine, spiperone, sufentanil, sulpiride, sumatriptan, tandospirone, terbutaline, terguride, testosterone, testosterone acetate, estosterone enanthate, testosterone proprionate, tetrahydrocannabinol, thioridazine, thiothixene, tiagabine, tianeptine, timolol, tiotropium bromide monohydrate, tizanidine, tobramycin, tofenacin, tolcapone, tolfenamate, tolfenamicacid, topiramate, tramadol, tranylcypromine, trazadone, triamcinolone acetonide, triethylperazine, trifluoperazine, trifluperidol, triflupromazine, trihexyphenidyl, trimeprazine, trimethobenzamide, trimipramine, tropisetron, tryptophan, valproicacid, vardenafil, venlafaxine, verapamil, vigabatrin, viloxazine, yohimbine, zafirlukast, zalospirone, zanamivir, zileuton, ziprasidone, zolmitriptan, zolpidem, zopiclone, zotepine, zuclopenthixol, and combinations and derivatives thereof.

In some embodiments, the pharmaceutical agent delivery and biometric data acquisition devices of the present disclosure can be used to administer unapproved drugs, pre-approved drugs, and/or drugs subject to clinical trials. For example, the devices can be used to assess the efficacy of various pharmaceutical and biological agents that are being evaluated in the context of a clinical trial. Test subjects can use the device in conjunction with clinical research being conducted to evaluate a drug's ability to attain or not attain certain clinical outcomes. The device can facilitate the acquisition of biometric data from the test subjects, as well as the aggregation of that data, in an effort to evaluate whether an experimental drug has therapeutic potential.

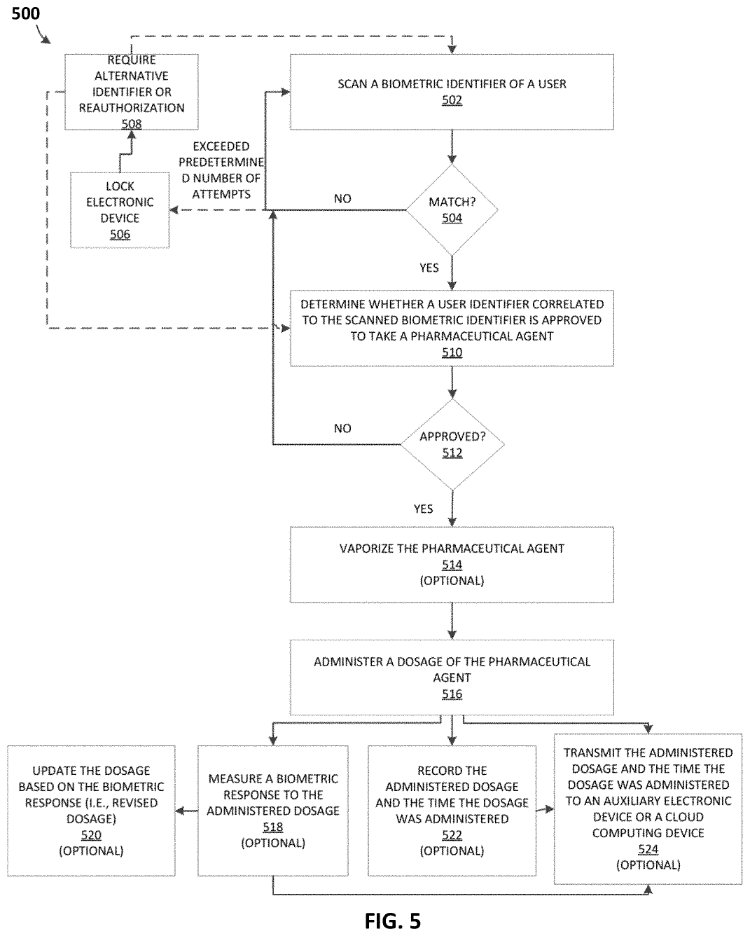

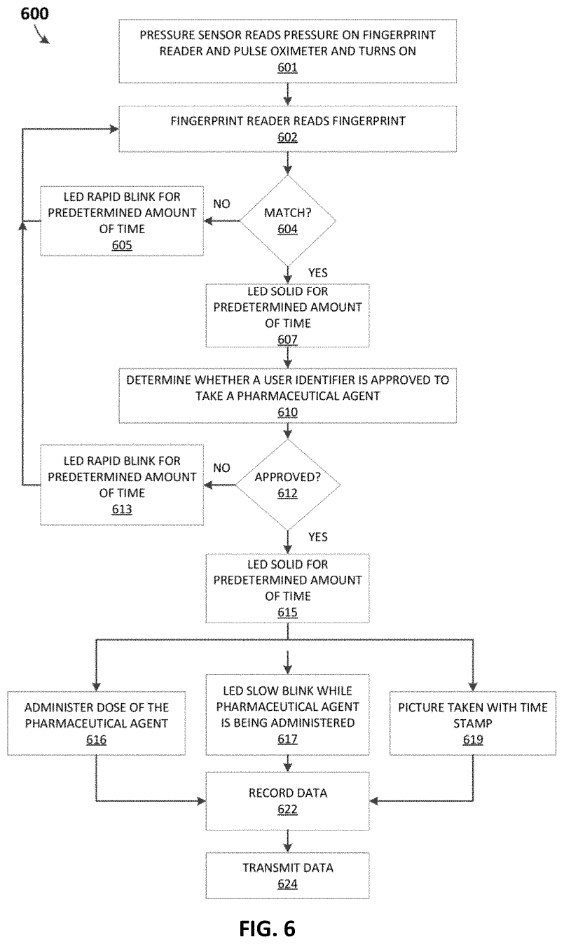

FIG. 5 is a flow diagram of an exemplary method 500 for administering a pharmaceutical or biological agent. In exemplary embodiments, the pharmaceutical agent delivery and biometric data acquisition device discussed throughout method 500 can have some or all of the same characteristics as the pharmaceutical agent delivery and biometric data acquisition device 100 described above in FIGS. 1-4. The pharmaceutical agent delivery and biometric data acquisition device will also be referred to herein as the “biometric data acquisition device.” For example, the pharmaceutical agent delivery and biometric data acquisition device can be turned on by holding a button (e.g., a fingerprint reader and/or pulse oximeter incorporated into the pharmaceutical agent delivery and biometric data acquisition device) for a predetermined amount of time, or by blowing air into or sucking air through the device. As another example, the pharmaceutical agent delivery and biometric data acquisition device can provide sensory feedback to a user intermittently during method 500. Examples of sensory feedback include, but are not limited to: visual cues, haptic feedback, or auditory feedback. As another example, the pharmaceutical agent delivery and biometric data acquisition device can take an image of the user intermittently during method 500. An example of sensory feedback is discussed in more detail in relation to FIG. 6 below. As another example, the pharmaceutical agent delivery and biometric data acquisition device can have wired and wireless connectivity. As another example, the pharmaceutical agent delivery and biometric data acquisition device can measure biometric responses of a user. This list, however, is not inclusive and, therefore, not meant to be limiting.

Method 500 begins by sensing a biometric identifier of a user using a pharmaceutical agent delivery and biometric data acquisition device (block 502). In exemplary embodiments, the biometric identifier includes, but is not limited to, the following: a fingerprint pattern, an iris pattern, a retina pattern, a vocal pattern, a facial-feature pattern, a pore pattern, a thermal image pattern, and a blood vessel pattern. The biometric data acquisition device can be equipped with various sensors and software to measure one or more of these biometric identifiers, as described above. In some embodiments, method 500 can begin when one or more audio sensors detects one or more audio signals from an authorized user, including the patient himself, or an authorized caregiver.

Next, at block 504, a determination can be made whether the scanned biometric identifier matches a stored biometric identifier. The stored biometric identifier can be an approved user's biometric identifier. In some exemplary embodiments, a stored biometric identifier can be securely stored in the pharmaceutical agent delivery and biometric data acquisition device's memory. Furthermore, in some exemplary embodiments, a stored biometric identifier can be concurrently securely stored on an auxiliary electronic device (e.g., a smartphone or a cloud computing device) that the pharmaceutical agent delivery and biometric data acquisition device can connect to, either wired or wirelessly. Or, in some other exemplary embodiments, the stored biometric identifier is not stored in the pharmaceutical agent delivery and biometric data acquisition device's memory, but only stored on an auxiliary electronic device, which the device can connect to, either wired or wirelessly. In exemplary embodiments, the stored biometric identifier can be included in a secure database of stored biometric identifiers. In exemplary embodiments, biometric identifiers for more than one user can be stored in the secure database of biometric identifiers and more than one biometric identifier for each user can be stored in the secure database of biometric identifiers.

In order to populate a list of stored biometric identifiers, an enrollment process can be undertaken. The enrollment process may include determining what biometric identifiers are to be used in method 500, enrolling each of those biometric identifiers using an iterative process, so that a fingerprint pattern, retina pattern, etc. can be recognized from various angles and under different conditions, and storing the enrollment data in the memory of the pharmaceutical agent delivery and biometric data acquisition device, or an auxiliary electronic device.

If the scanned biometric identifier matches a stored biometric identifier at block 504, then the method 500 continues to block 510. If the scanned biometric identifier does not match a stored biometric identifier, then method 500 can proceed back to scanning the biometric identifier at block 502. However, in some exemplary embodiments, if method 500 proceeds to scan a biometric identifier a predetermined number of times but is unable to match the scanned biometric identifier to a stored biometric identifier, then method 500 can proceed to locking the biometric data acquisition device at block 506. The predetermined number of times can be configurable when setting up the biometric data acquisition device. In some exemplary embodiments, method 500 will try to match the scanned biometric identifier to a stored biometric identifier three times before locking the biometric data acquisition device. In some other exemplary embodiments, method 500 will attempt to match the scanned biometric identifier to a stored biometric identifier five times before locking the biometric data acquisition device. In yet other exemplary embodiments, method 500 will attempt to match the scanned biometric identifier to a stored biometric identifier an unlimited number of times without locking the biometric data acquisition device.

In the embodiments where method 500 proceeds to block 506 and locks the biometric data acquisition device, method 500 can proceed to block 508 and require either an alternate identifier or reauthorization to unlock the pharmaceutical agent delivery and biometric data acquisition device. In some exemplary embodiments where method 500 requires an alternative identifier at block 508, a different biometric identifier than the one previously scanned may be scanned and matched to a stored biometric identifier in order to unlock the biometric data acquisition device. For example, if the biometric identifier initially scanned by the biometric data acquisition device was a fingerprint, then a user's retina may be scanned and matched to a stored biometric identifier in order to unlock the biometric data acquisition device. Or, as another exemplary embodiment, a passcode may be entered into the biometric data acquisition device in order to unlock the biometric data acquisition device. In other embodiments, block 508 may require reauthorization of the biometric data acquisition device by the manufacturer of the device, a certified healthcare professional or other third-party.

Once the biometric data acquisition device is unlocked, in some embodiments, method 500 can proceed back to block 502 or to block 510, depending on how the biometric data acquisition device was unlocked. For example, if a passcode was entered, method 500 may proceed back to 502 to identify a biometric identifier of a user since a biometric identifier was never matched to a stored biometric identifier. As another example, if a retina was scanned and the retina pattern is matched to a stored biometric identifier to unlock the biometric data acquisition device, method 500 may proceed to block 510 since a biometric identifier was matched to a stored biometric identifier. In yet another example, if the biometric data acquisition device was unlocked by the manufacturer, a healthcare professional or other third-party, the person or entity that unlocked the biometric data acquisition device may determine whether method 500 proceeds to block 502 or to block 510.

In embodiments where the scanned biometric identifier matches a stored biometric identifier at block 504, then method 500 may determine, using the biometric identifier, whether the user is approved to take a pharmaceutical agent of a list of approved pharmaceutical agents for example, at block 510. In certain embodiments, determining whether the user is approved to take a pharmaceutical agent can include matching the scanned biometric identifier to a stored biometric identifier, wherein the stored biometric identifier is an approved user's biometric identifier. Additionally, in exemplary embodiments, each stored biometric identifier can be correlated to a user identifier of the user. The user identifier may be the name of the user, the social security number of the user or rendition thereof, a username of the user, or a random number assigned to the user during configuration of the pharmaceutical agent delivery and biometric data acquisition device. A random number or username may be used to protect the privacy of the user, in addition to the biometric identifier being correlated to a user identifier.

In addition to being correlated to a biometric identifier, the user can be linked to a list of pharmaceutical agents that are eligible to be taken by the user based on for example, medical history of the user. In exemplary embodiments, the list of pharmaceutical agents may include all the pharmaceutical agents currently and previously prescribed to the user of the user identifier. If the user has never been prescribed a pharmaceutical agent, the list of prescribed pharmaceutical agents can be the null set. In some embodiments, the list of prescribed pharmaceutical agents can be uploaded to the device by a healthcare professional. This can be done either remotely when the biometric data acquisition device is either wired or wirelessly connected to a network or when the biometric data acquisition device is in the presence of a healthcare professional. In some exemplary embodiments, the list of pharmaceutical agents may include over-the-counter pharmaceuticals, nutraceuticals, minerals, supplements, vitamins, and the like.

In addition to correlating a potential list of pharmaceutical agents that are available for use by the user (prescribed, monitored and non-prescribed) for a particular purpose or in general, determining whether the user affiliated with the user identifier is approved to take a target pharmaceutical agent may include determining the present time (realtime) and date during which the biometric identifier is scanned, when the last time or any previous time a biometric identifier was scanned or when the pharmaceutical agent was administered to the user and based in part on this information, whether the instant time and/or date is within a time period that the pharmaceutical agent is allowed, safe or optimal to be taken.

If a user is approved to take a pharmaceutical agent, then the biometric data acquisition device can give sensory feedback (e.g. visual or audio signal) to the user of the user identifier that the user is approved to take a pharmaceutical agent. In some exemplary embodiments, to determine whether a specific or family of pharmaceutical agents or monitored agents are approved to be taken by the user of the user identifier, the agent may be associated with the biometric data acquisition device by an authenticated user or an approved caregiver or healthcare provider. Then, the pharmaceutical agent delivery and biometric data acquisition device can determine whether the coupled pharmaceutical agent is approved to be taken by the user. The pharmaceutical agent delivery and biometric data acquisition device can determine what pharmaceutical agent is associated with the biometric data acquisition device. In accordance with these embodiments, this can be determined in a variety of ways including, but not limited to, radio-frequency identification (RFID), resistance sensing, barcode scanning, etc. In some other exemplary embodiments, to determine whether a specific pharmaceutical agent is approved to be taken by the user, the pharmaceutical agent delivery and biometric data acquisition device can include an input and sensory feedback device for selecting a specific or family of pharmaceutical agents from a list of pharmaceutical agents. Similar to blocks 502-510, sensory feedback can be given to the user throughout the process of determining whether a user is approved to take a pharmaceutical agent.

In some embodiments, stored biometric information can be used to determine whether one or more of a subject's current biometrics is anomalous or abnormal and whether this observation can be connected to administration of a particular pharmaceutical, monitored or biological agent. For example, a subject's biometric data can be acquired and stored on the device or an auxiliary electronic device. If a subject's specific instantaneous biometric response is outside a certain usage range, which has been established by the subject's recent history of biometric responses aggregated together, an alarm may be triggered by the device, even if the biometric response has been determined to be within an acceptable, previously determined range (e.g., a clinical range determined from patient trials). In this way, the device can be customized to operate according to a subject's individualized biometric responses.

At block 512, for example, if the user identifier is approved to take a pharmaceutical agent of interest to treat a disease or condition, method 500 may proceed to block 514 or block 516. On the other hand, if the user identifier is not approved to take the pharmaceutical agent of interest, then method 500 may proceed to block 502 or the method 500 may end. Similar to the blocks above, sensory feedback can be given to the user as to whether method 500 is proceeding to block 502, block 514, block 516, or method 500 is ending. Depending on the feedback, a user where the pharmaceutical agent is not approved can receive feedback to contact their physician or seek alternative assistance.

In some embodiments, method 500 proceeds to block 514 where the approved pharmaceutical agent is vaporized by the pharmaceutical agent delivery and biometric data acquisition device. In some of these embodiments, the pharmaceutical can be vaporized used ultrasonic frequencies that heats and/or cavitates and vaporizes the pharmaceutical agent. In other embodiments, the pharmaceutical agent delivery and biometric data acquisition device vaporizes the pharmaceutical agent by heating it. In exemplary embodiments, the pharmaceutical agent can be heated to between 300° F. and 500° F. in order to vaporize the pharmaceutical agent. In exemplary embodiments, sensory feedback can be given to the user when the pharmaceutical agent delivery and biometric data acquisition device is ready to vaporize the pharmaceutical agent, when the pharmaceutical agent delivery and biometric data acquisition device is vaporizing the pharmaceutical agent, and when the pharmaceutical agent delivery and biometric data acquisition device is finished vaporizing the pharmaceutical agent. In addition, sensory feedback can be given to the user when the vaporized pharmaceutical agent is cleared from the delivery and biometric data acquisition device and safe for storage.

In other exemplary embodiments, if a pharmaceutical agent is approved at block 512 method 500 may proceed from block 512 to block 516. At block 516, a dosage of the pharmaceutical agent can be administered via a mouthpiece of the pharmaceutical agent delivery and biometric data acquisition device. In some exemplary embodiments, administering a dosage of the pharmaceutical agent includes, but is not limited to, identifying a pharmaceutical agent associated with the biometric data acquisition device, and determining whether the pharmaceutical agent matches the approved pharmaceutical agent.

In another exemplary embodiment, administering a dosage through a mouthpiece associated with the device can include, but is not limited to, identifying a pharmaceutical agent associated with the biometric data acquisition device; determining whether the pharmaceutical agent matches the approved pharmaceutical agent; commencing administration of the pharmaceutical agent through the mouthpiece of the pharmaceutical agent delivery and biometric data acquisition device, measuring the amount of pharmaceutical agent administered through the mouthpiece of the pharmaceutical agent delivery and biometric data acquisition device; and ceasing administration of the pharmaceutical agent when administration of the predetermined dosage has completed. In accordance with this method, the administration methods can further include repeating this process for all subsequent dosages administered to the subject and include recording biometric data associated with the subsequent dosages. In certain embodiments, the recorded biometric data can be used to evaluate, for example, whether the subject (or healthcare provider administering the pharmaceutical agent) is in compliance with a predetermined treatment plan.

In some embodiments, method 500 may continue to block 518 and where biometric response can be measured related to the dosage administered or the regimen determined for the user. In some embodiments, biometric response of the user can be measured relatively soon or immediately after the pharmaceutical agent has been administered or after a period of time has lapsed. In some embodiments, biometric response of the user can be performed periodically. In other embodiments, the pharmaceutical agent delivery and biometric data acquisition device can be equipped with various sensors to measure one or more of the following biometric responses, for example to evaluate the progress or response of the subject regarding his/her condition: a galvanic skin response, a blood oxygen level response, a body temperature response, a heartrate response, a perfusion index, a blood pressure response, a retina response, an eye movement response, eye color (e.g., yellowing of the sclera), an inhalation velocity response, an inhalation pressure response, an inhalation volume response, an expiratory velocity response, an expiratory pressure response, an expiratory volume response, or an exhale chemical composition response. This list, however, is not inclusive and, therefore, is not meant to be limiting.

In some embodiments, where a biometric response is measured by the pharmaceutical agent delivery and biometric data acquisition device, method 500 can proceed to block 520 where the dosage is updated to generate a revised dosage based on measured biometric response(s). In other embodiments, a pharmaceutical or other monitored agent dose or frequency of administration can be revised by a healthcare professional after the information regarding a previous dosage, the time of a previous dosage, the frequency of a previous dosage and the biological response to a previous dosage or other biometric data has been transmitted and evaluated by the healthcare professional.

In some exemplary embodiments, method 500 may continue to block 522 and record, on the biometric data acquisition device's memory, each time a dosage was administered to a particular user, amount of the dosage, time of day the dosage was administered and date of the dosage administered. In certain aspects, all dosages subsequent to an administered dosage, including revised and unrevised dosages, can be recorded on the device and used to evaluate a user's progress as well as adherence to a pre-determined treatment plan. In some embodiments, this information can be transmitted to an auxiliary electronic device for storage, transport or evaluation etc.

For each of blocks 516, 518, 522, amount of administered dosage, time of the dosage, frequency of the administered dosage, whether or not the dosage was revised, and any other information that may be pertinent in order to monitor treatment of the user can be transmitted to an auxiliary electronic device. The information can be transferred using a wired connection or a wireless connection if and when one becomes available. In some embodiments, until a network connection becomes available, the information can be stored on the pharmaceutical agent delivery and biometric data acquisition device's memory.

FIG. 6 is a flow diagram representing a method 600 illustrating one example of method 500. Method 600 serves as an example and is not meant to be limiting. In embodiments where a user is visually impaired, any visual cues (e.g., the LED lights) in method 600 can be replaced with other sensory feedback (e.g., auditory or haptic feedback). Method 600 begins by turning on the pharmaceutical agent delivery and biometric data acquisition device at block 601. In some embodiments, this can be done by holding down on a fingerprint reader and pulse oximeter included in the pharmaceutical agent delivery and biometric data acquisition device for a predetermined amount of time. For example, holding down on the fingerprint reader and pulse oximeter for 5 seconds may turn the pharmaceutical agent delivery and biometric data acquisition device on.

Once the pharmaceutical agent delivery and biometric data acquisition device is on, method 600 can proceed to block 602 where a fingerprint of a user is scanned by a fingerprint scanner that can be included in the pharmaceutical agent delivery and biometric data acquisition device. At block 604, if the scanned fingerprint does not match a stored fingerprint, then method 600 proceeds to block 605, at which time an indicator, such as a rapidly blinking LED notifies the user that the scanned fingerprint did not match a stored fingerprint. Method 600 then proceeds back to block 602 to allow the user to scan their fingerprint again. If, however, the scanned fingerprint matches a stored fingerprint, then method 600 proceeds to 607, at which time a different indicator, such as a solidly illuminated LED notifies the user that their scanned fingerprint matches a stored fingerprint.

At block 610, method 600 proceeds by determining (e.g. by stored information) whether the user correlated to the scanned and matched fingerprint is approved to take a particular pharmaceutical or other monitored agent. As detailed above, this may include determining the current time and date, when the last time the pharmaceutical agent was administered to the user and whether the current time and date is within an allowable or recommended time period for the user to administer a dose of the pharmaceutical agent.

At block 612, if a determination is made that the user is not approved to take a pharmaceutical agent, then method 600 may proceed to block 613, at which time an indicator such as a rapidly blinking LED or audio notifies the user that the a pharmaceutical agent has not been approved for the user to take at the time of the fingerprint read. If method 600 does proceed to block 613 because the user is not approved to take a pharmaceutical agent, then method 600 can revert back to block 602 or method 600 can end. If, a determination is made that the user being assessed is approved to take a pharmaceutical or other monitored agent at block 612, then method proceeds to block 615, at which time an indicator such as a solidly illuminated LED or audio signal notifies the user that a pharmaceutical agent has been approved to be taken by the user.

After block 615, method 600 proceeds to blocks 616, 617, and/or 619 which can occur concurrently or within a prescribed time period of one another. At block 616, a dosage of the pharmaceutical agent is administered by the pharmaceutical agent delivery and biometric data acquisition device. The dosage can be administered as describe above in method 500. While the pharmaceutical agent is being administered, and in some embodiments before, an LED or audio can slowly blink or sound-off to notify the user that the pharmaceutical agent is being administered or about to be administered at block 617. When the administration of the pharmaceutical agent is completed, the LED can stop blinking or the audio shuts off. Concurrently with the administration of the pharmaceutical agent, the pharmaceutical agent delivery and biometric data acquisition device can be configured to take a picture (e.g. for identification or assessment of after effects etc.) of the user at block 619. In some embodiments, a timestamp can be included with the picture, so that the time that the pharmaceutical agent was administered can be recorded along with a record number and one or more user identifiers (e.g., user's picture).

In another embodiment, method 600 can then proceed to block 622 where the data from administration can be recorded to memory pharmaceutical agent delivery and biometric data acquisition device. In some embodiments, the data recorded can be any of the data discussed above in method 500. Examples include, but are not limited to, pharmaceutical or monitored agent, dose administered of the pharmaceutical agent, time and date of the administration of the pharmaceutical agent, and the biometric response to the administration of the biological agent. In other embodiments, other agents (e.g. over-the-counter agents, vitamins) taken or used by a user can also be recorded by the user using a recorder on the device or other method for recordation in realtime or at a later time by the user.

Method 600 can proceed to block 624 where the recorded data can be transmitted to a secondary electronic device, a cloud computing device or an application stored therein while backed-up by the device. Various user identifiers can be associated with a user's biometric data stored on an electronic record, such that the user can access the biometric data using his/her user identifier. In some embodiments, the user's biometric data is uploaded and stored in a cloud computing device that can be accessed using one or more user identifiers.

### Monitoring Delivery Parameters of an Administered Pharmaceutical Agent

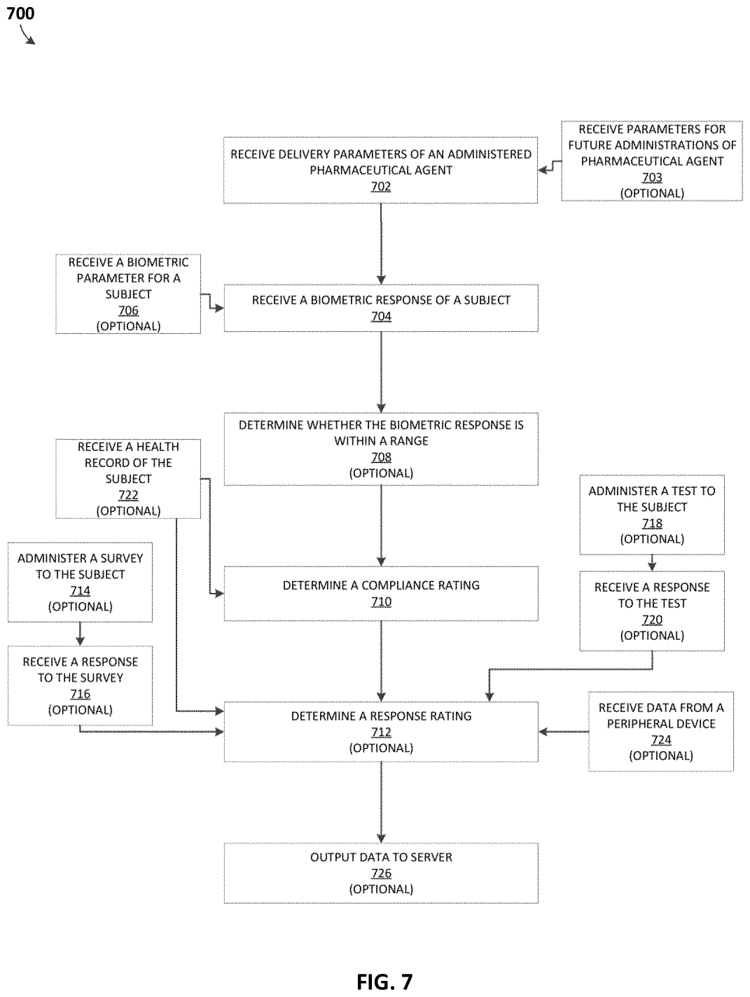

FIG. 7 represents an exemplary method 700 by flow diagram for monitoring delivery parameters of an administered pharmaceutical agent. In embodiments, method 700 may be used with a monitored device such as, but not limited to, the pharmaceutical delivery and biometric monitoring device described above, the pharmaceutical and biological agent desktop dispensing system having biometric data acquisition and monitoring capabilities described in U.S. Provisional Patent Application Ser. No. 62/212,441, the solid pharmaceutical agent dosage form dispensing and biometric data acquisition device described in U.S. Provisional Patent Application Ser. No. 62/191,972, the nebulizing devices and systems having biometric data acquisition and monitoring capabilities described in U.S. Provisional Patent Application Ser. No. 62/191,974, and the pharmaceutical and biological agent delivery system having biometric data acquisition and monitoring capabilities described in U.S. Provisional Patent Application Ser. No. 62/068,648, U.S. Provisional Patent Application Ser. No. 62/145,399 and U.S. Provisional Patent Application Ser. No. 62/191,979.

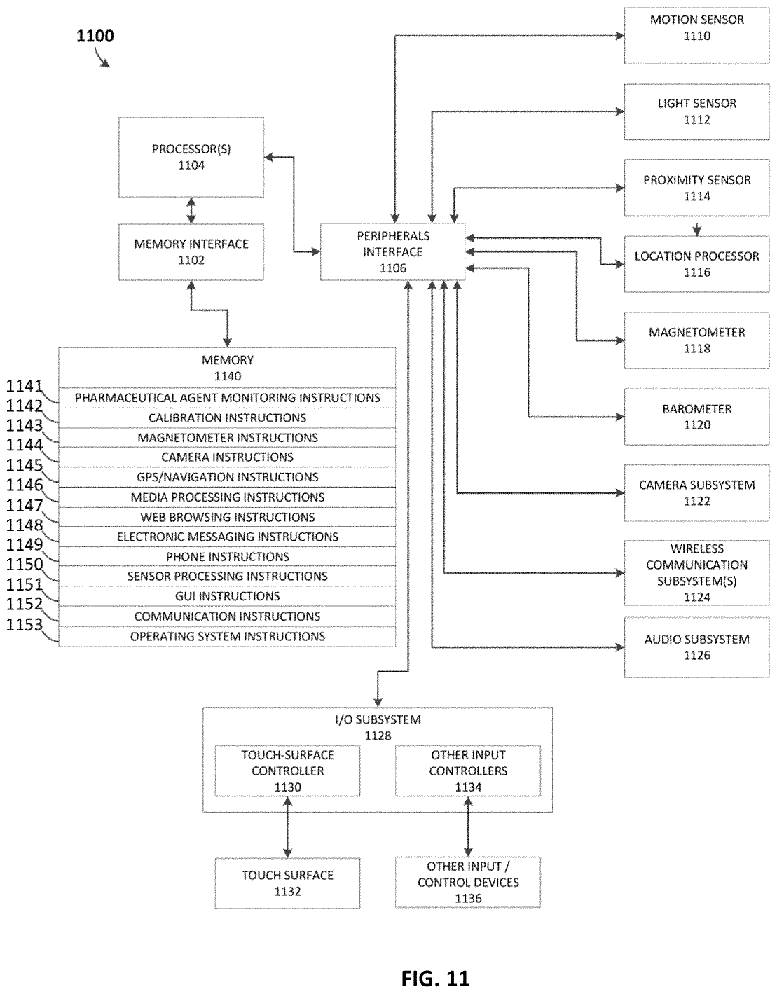

Method 700 includes receiving, from a pharmaceutical agent and biometric data acquisition device, delivery parameters of an administered pharmaceutical agent to a user (block 702). In certain embodiments, the delivery parameters can be received by a computing device. As presented above, the delivery parameters can be monitored by the pharmaceutical agent and biometric data acquisition device and stored in the memory of the pharmaceutical agent and biometric data acquisition device. In other embodiments, the delivery parameters can be received (e.g., by a computing device) after the pharmaceutical agent and biometric data acquisition device sends, via a wired or wireless connection, the delivery parameters. One exemplary computing device is described below and is found in representative FIG. 11.

In exemplary embodiments, the delivery parameters of the administered pharmaceutical agent can include, but are not limited to, one or more of the following: a dose of the pharmaceutical agent administered to the user, a time at which the pharmaceutical agent was administered to the user, and a history of administration of the pharmaceutical agent to the user. The history of the administration of the pharmaceutical agent can include, but is not limited to, one or more of the following: how long the user has been receiving the pharmaceutical agent (e.g., days, months, years, etc.), frequency of the user receiving the pharmaceutical agent (e.g., twice daily, daily, weekly, etc.), the time of day that the pharmaceutical agent was administered and previous administrations of the pharmaceutical agent.

In some embodiments, method 700 can further include receiving, from a pharmaceutical agent and biometric data acquisition device, parameters for future administrations of the pharmaceutical agent (block 703). In certain embodiments, parameters for future administrations can be received by a computing device. Parameters for future administrations or delivery of the pharmaceutical agent can include, but are not limited to, one or more of the following: duration of administration of the pharmaceutical agent, frequency of administration of the pharmaceutical agent and the dosage for the future administrations of the pharmaceutical agent.

Method 700 further comprises receiving, from the pharmaceutical agent delivery and biometric data acquisition device, at least one biometric response of the user (block 704). In some embodiments, the biometric response can be received by a computing device. The biometric response of the user can be response due to receiving a dose of the pharmaceutical agent and can be obtained for example, by using the sensors described above in FIGS. 1-6 or other sensor known in the art. For example, if the pharmaceutical agent is an agent meant to relax the user, an example of a biometric response can be a reduced heart rate or reduced blood pressure measured by a sensor of the pharmaceutical agent delivery and biometric data acquisition device. In some embodiments, the at least one biometric response to the pharmaceutical agent can be measured by a sensor of the pharmaceutical agent delivery and biometric data acquisition device during at least one of the following intervals: less than five minutes after taking the pharmaceutical agent, less than one hour after taking the pharmaceutical agent, less than one day after taking the pharmaceutical agent, less than one week after taking the pharmaceutical agent or less than one month after taking the pharmaceutical agent.

In certain embodiments, the at least one biometric response can include, but is not limited to, the following: a galvanic skin response, a blood oxygen level response, a body temperature response, a heartrate response, a perfusion index response, a blood pressure response, a retina response, an eye movement response, an inhalation velocity response, an inhalation pressure response, an inhalation volume response, an expiratory velocity response, an expiratory pressure response, an expiratory volume response or an exhale chemical composition response.

The biometric responses can be stored in memory of the pharmaceutical agent delivery and biometric data acquisition device. In some embodiments, the biometric responses can be received by a computing device after the pharmaceutical agent and biometric data acquisition device sends, via a wired or wireless connection, the biometric responses to the computing device.

In other embodiments, method 700 further includes receiving at least one biometric parameter for a user, wherein the at least one biometric parameter for the user can be measured by the pharmaceutical agent delivery and biometric data acquisition device before the pharmaceutical agent is administered to the user (block 706). The biometric parameter can be measured by the pharmaceutical agent delivery and biometric data acquisition device at different times before the pharmaceutical agent is administered to the user. For example, the biometric parameter can be measured five minutes before the pharmaceutical agent is administered, more than one hour before the pharmaceutical agent is administered, or more than one day before the pharmaceutical agent is administered.

In embodiments, method 700 may also include diagnosing and/or flagging possible medical disorders using one or more of the at least one biometric parameters before the pharmaceutical agent is administered to the user (block 706) and/or the at least one biometric parameters after the pharmaceutical agent is administered to the user (block 704). For example, measuring biometric parameters for a user's may lead to a diagnosis for glaucoma. As another example, assume a pharmaceutical agent is prescribed by a physician to treat a first medical disorder. If the prescribed pharmaceutical agent is known to affect users with a second medical disorder, that was not previously diagnosed, in a certain manner that can be determined using one or more of the at least one biometric parameters that are measured, and; a user elicits the one or more biometric parameters indicating that the user has the second medical disorder, method 700 may include indicating to the user that the user has, or may have, the medical disorder. However, these are only examples and not meant to be limiting.

A biometric parameter can be stored in the memory of the pharmaceutical agent delivery and biometric data acquisition device. In some embodiments, the biometric parameters can be received by a computing device after the pharmaceutical agent and biometric data acquisition device sends, via a wired or wireless connection, the biometric responses to the computing device or transferred via chip, disk or USB.

In certain embodiments, the biometric parameter can include, but is not limited to, the following: blood oxygen level, body temperature, heartrate, perfusion index, blood pressure, inhalation velocity, inhalation pressure, inhalation volume, expiratory velocity, expiratory pressure, expiratory volume or exhale chemical composition.

In other embodiments, method 700 can further include determining whether the biometric response from block 704 or the biometric parameter from block 706 or both is within a range (e.g. predetermined favorable range) (block 708). A range can be initialized by the user or a third-party such as a healthcare provider. For example, a biometric parameter of heart rate can have a favorable range of 60 beats per minute (bpm) and 90 bpm. Method 700 can determine whether the heart rate, as sensed by a sensor of the pharmaceutical agent and biometric data acquisition device, is within that range. If the user's heart rate is not within a predetermined range, then in some embodiments, method 700 can further include sending an alert to the user or a third-party or both in order. If the user's heart rate is within the predetermined range, then an alert is not sent in embodiments. However, the user can still be notified using some other indication, such as a checkmark being displayed on the screen of a computing device. The same steps can be performed by method 700 for a biometric response.

Method 700 can further include determining, using a computing device, a compliance rating using at least one of the following: the delivery parameters of the administered pharmaceutical agent and the at least one biometric response (block 710).

In some exemplary methods, to determine a compliance rating, the dosing regimen for the administered pharmaceutical agent can be uploaded to the computing device. The dosing regimen can include, but is not limited to, the type of pharmaceutical agent, the frequency with which a user is supposed to take the pharmaceutical agent (hourly, twice daily, daily, etc.), the time of day the pharmaceutical agent is supposed to be taken by the user, the duration that the user is supposed to take the pharmaceutical agent (e.g., days, weeks months, etc.), and the dosage of the pharmaceutical agent that is to be administered each time. In some embodiments, the computing device can determine whether the delivery parameters of the administered pharmaceutical agent from block 702 match or are close to the dosing regimen. For example, were the delivery parameters (e.g., time, frequency, duration and dosage) of the pharmaceutical agent similar to or match the dosing regimen (e.g., time, frequency, duration and dosage). For example, a prophetic dosing regimen for a pharmaceutical agent can be the following: 1 mg, taken daily in the morning for a period of two weeks. At the completion of the two-week period, the delivery parameters indicate that 13 out of the 14 days 1 mg of the pharmaceutical agent was taken once a day between the time of 6 am and 8 am. Then, in this example, the computing device may determine a compliance rating of 13/14≈93%. If, however, in another scenario, that the delivery parameters indicated that 12 out of 14 days 1 mg of the pharmaceutical agent was taken once a day between the time of 6 am and 8 am and on 2 separate days out of 14 days 1 mg of the pharmaceutical agent was taken between the time of 3 pm and 4 pm, then the compliance rating of 93% can be adjusted down accordingly to, for example, 85% etc. In this example, depending on the type of pharmaceutical agent and how relevant the time of day that the pharmaceutical agent is taken or duration between doses etc., compliance rating can be adjusted up or down more or less, depending on whether the delivery parameters indicate that the pharmaceutical agent was not taken during the appropriate time of day. If a higher dosage or a lower dosage of the pharmaceutical agent is administered, the compliance rating can be adjusted accordingly.

In other embodiments, the compliance rating can also be determined using the biometric response. For example, assume a dosing regimen for a pharmaceutical agent is the following: 1 mg, taken daily in the morning for a period of two weeks. Also assume that after 1 mg of the pharmaceutical agent is taken by a user, the user's blood pressure is supposed to decrease by 10%. Then, in this example, if a biometric response is received from the pharmaceutical agent delivery and biometric data acquisition device that a user's blood pressure decreased by 8%-12% in the morning between 6 am and 8 am for 13 out of 14 days during the two week period that the pharmaceutical agent was supposed to be administered to the user, then a compliance rating of 93% can similarly be assigned. In this example, if the biometric response is recorded during another time of day, less frequency or by a greater or smaller magnitude than predicted, then the compliance score can be adjusted up or down accordingly.

In some embodiments, both the delivery parameters and the biometric response are used to determine a compliance rating by the computing device. For example, a dosing regimen for a pharmaceutical agent can be 1 mg, taken daily in the morning for a period of two weeks. After the dose is taken by the user, the user's blood pressure is supposed to decrease by 10% in this example. Then, in this example, the delivery parameters recorded indicate that 13 out of the 14 days 1 mg of the pharmaceutical agent was taken once a day between the time of 6 am and 8 am. However, a biometric response was received that indicated only 12 out of 14 days that a user's blood pressure decreased by 8%-12% in the morning between 6 am and 8 am. In this example, the computing device can compute a decrease in the compliance rate. This may be due to the user not properly receiving or administering the pharmaceutical agent either by choosing not to take an administered dosage or by improperly using the pharmaceutical agent and biometric data acquisition device. Compliance ratings calculated in the above three examples are for illustrative purposes only. Other methods may be used to determine a compliance rating.

In certain embodiments, method 700 can further include determining, using a computing device, a response rating using the at least one biometric response (block 712). In some embodiments, the response rating can be determined by comparing an expected biometric response to an actual or recorded biometric response. For example, after 1 mg of the pharmaceutical agent is taken by a user, the user's blood pressure is supposed to decrease by 10%. Assume that after the pharmaceutical agent is taken by the user that the user's blood pressure decreases by 9.5%. In this example, the response rating may be determined to be 1−|9.5−10|/10=95%.

In some embodiments, method 700 can further include providing a survey to the user (block 714) and receiving at least one response to the survey (block 716). In certain embodiments, the response can be used in determining response rating from block 712. The survey can be administered to the user during different time periods. For example, in some embodiments, the survey can be provided to the user before the pharmaceutical agent is administered. In some examples, the survey can be provided to the user after the pharmaceutical agent has been administered. In some examples, the survey can be provided to the user before the pharmaceutical agent is administered and after the pharmaceutical agent is administered. For example, the survey can be provided immediately, ten minutes, an hour, four hours, eight hours or a day before/after the pharmaceutical agent has been administered, to suggest a few exemplary time periods.

The type of survey that can be provided can be dependent on the type of pharmaceutical agent. For example, if the pharmaceutical agent is a pain medication, then about 20 minutes to about one hour after the pain medication is administered, the user can be provided a survey that asks the user about his/her pain level. The response to the survey can help determine the response rating and, as a result, determine whether the pharmaceutical agent is effective at the current dose and whether the dosage needs to be adjusted. Additionally, the survey can be provided again at another time interval, for example, four hours after the pharmaceutical agent was administered. By re-administering the survey, it can be determined how long the pharmaceutical agent lasts and whether the frequency that the pharmaceutical agent is administered needs to be adjusted, either increased or decreased.

In some embodiments, method 700 further includes providing a test to the user (block 718) and receiving at least one response to the test (block 720). Similar to the survey above, in certain examples, response to the test can be used in determining the response rating from block 712. The test can be administered to the user during different time periods. For example, in some embodiments, the test can be provided to the user before the pharmaceutical agent is administered. In some examples, the test can be provided to the user after the pharmaceutical agent has been administered. In other examples, the test can be provided to the user before the pharmaceutical agent is administered and after the pharmaceutical agent is administered. For example, the test can be provided immediately, ten minutes, an hour, four hours, eight hours or a day before/after the pharmaceutical agent has been administered as is considered appropriate, to suggest a few exemplary time periods. Types of exemplary tests can include, but are not limited to, recognition tests, speed tests, reflex tests, memory tests and coordination tests.

The type of test provided can be dependent on the type of ailment that the pharmaceutical agent is trying to remedy. For example, if the pharmaceutical agent is supposed to diminish the effects of Alzheimer's, a memory test (e.g., the game Limo situ) can be provided to the user. As another example, if the pharmaceutical agent is supposed to increase someone's hand-eye coordination, a test that tests someone's hand-eye coordination can be administered to the user. The response to the test can help determine the response rating and, as a result, determine whether the pharmaceutical agent is effective and whether the dosage needs to be altered, increased or decreased. Similar to above, the test can be provided for at least a second time at a later time. The second test can help determine how long the pharmaceutical agent lasts in a subject and whether the frequency that the pharmaceutical agent is administered needs to be altered, increased and decreased.

In some embodiments, the tests can include interactive sounds and effects when you interact with the test (e.g., haptic feedback). Moreover, the tests can retain a history of scores and present the user with rewards when the user attains levels of proficiencies. In some of these embodiments, the user can be rewarded with electronic trophies, ribbons, different ranking levels (e.g., private, corporal, sergeant, lieutenant, etc. or level 1, 2, 3, etc.), and the like. Furthermore, in some embodiments, a user's results from a first device 100 can be linked to one or more user's results from another device and users can compete against one another based on their level of proficiencies on tests or compliance ratings. For example, if two users are being treated with a pharmaceutical agent that is supposed to increase the user's hand-eye coordination, a competition can be created that determines who has the most improved hand-eye coordination according to the users' respective results on the administered tests, over a 30-day time period. This feedback can increase the likelihood of retaining the user's attention and to encourage the user to continue to take the tests.

In other embodiments, method 700 can further include receiving a health record of the subject (block 722). In some embodiments, the health record of the subject can aid in determining personal tendencies of the patient. For example, if a typical response to a pharmaceutical agent results in a decreased blood pressure of 10%, but in the health history of the user an 8% decrease in blood pressure is more typical, the compliance rating and/or the response rating can be adjusted accordingly.

In some exemplary embodiments, method 700 can further comprise receiving data from peripheral device (block 724). The data received from the peripheral device can help determine the response rating from block 712. For example, if the pharmaceutical agent is a pain medication, the peripheral device (e.g., pedometer), can determine how much the user is able to walk around. If the pedometer indicates that there is little to no movement, then the response rating can be decreased accordingly in embodiments. Examples of peripheral devices can include, but are not limited to, the following: pedometers, heart rate monitors, blood oxygen sensors, body temperature sensors, and blood pressure monitors.

In some embodiments, method 700 can further include outputting the data (e.g., the response rating, the compliance rating, biometric responses, etc.) to a server (block 726). After the data is output to a server, the data can be integrated from multiple users and, in some embodiments, the data can be analyzed to determine how effective a pharmaceutical agent is at treating the disorder that the pharmaceutical agent is designed to treat. For example, if a pharmaceutical agent is designed to decrease the effects of Alzheimer's in 80% of users, but only 40% of the users are exhibiting better results on a memory test, the pharmaceutical agent can be determined to be not as effective as it was originally designed to be. As another example, if there are a multitude of users responding to an administered survey that their pain has not gone down significantly after being administered a pharmaceutical agent, then the pharmaceutical agent can be determined not to be an effective pain medication.

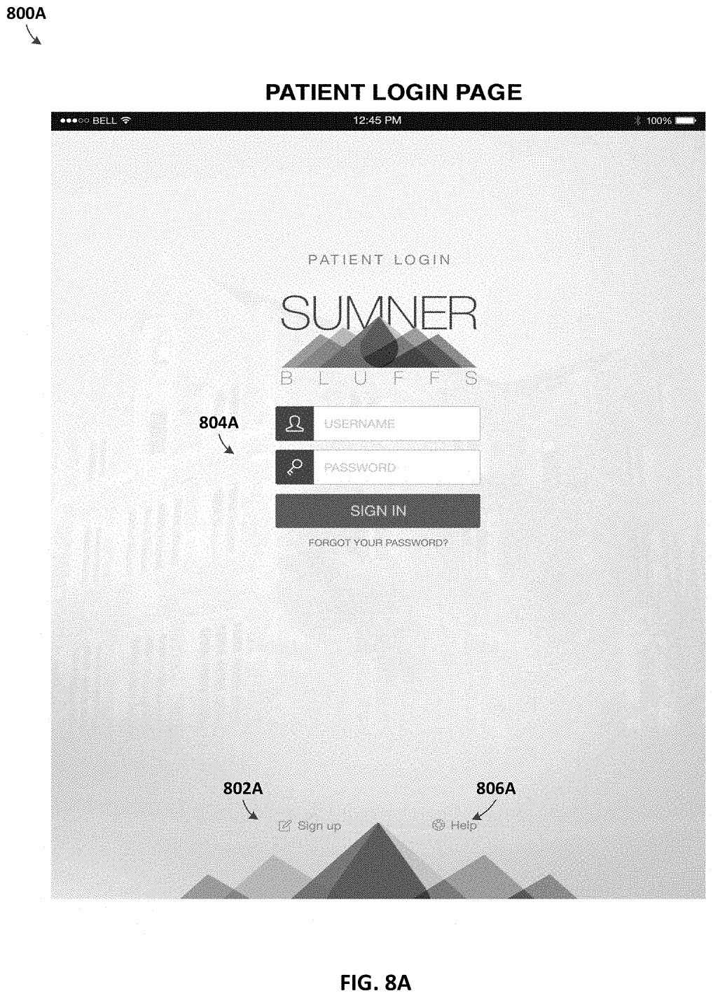

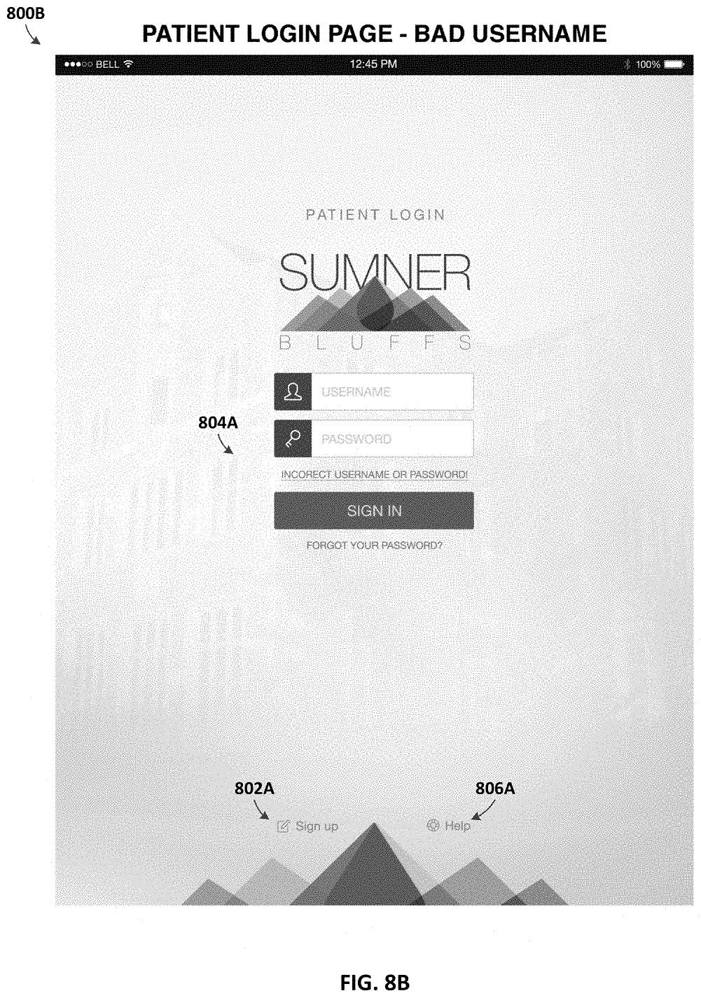

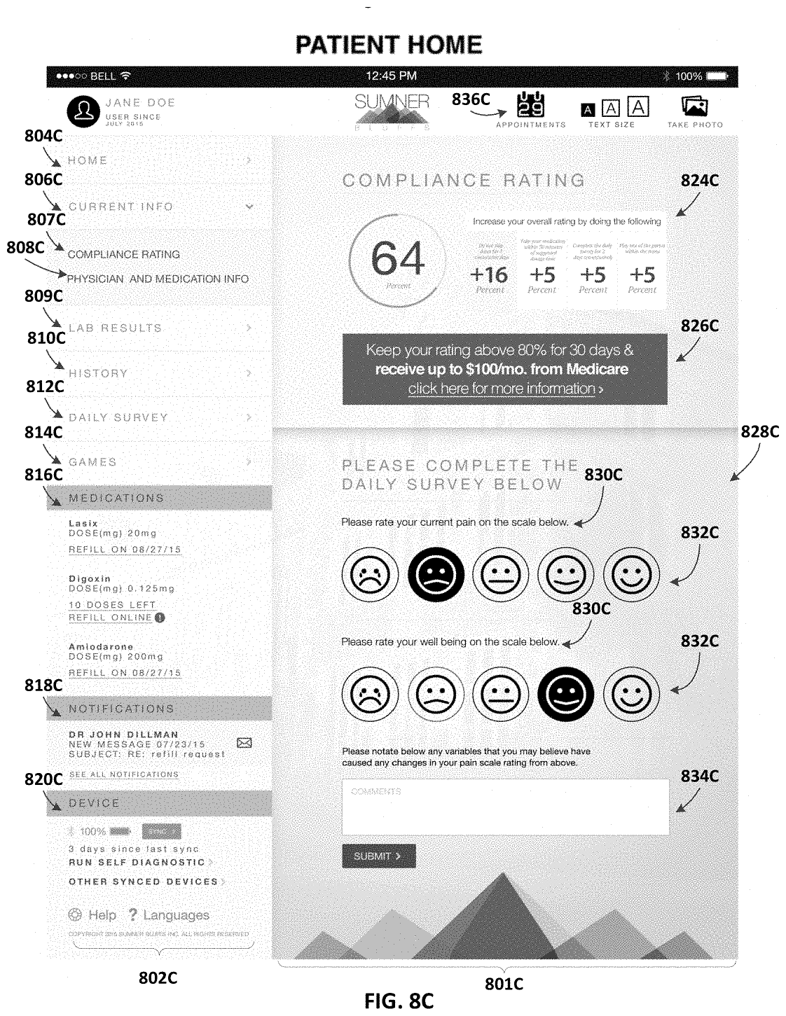

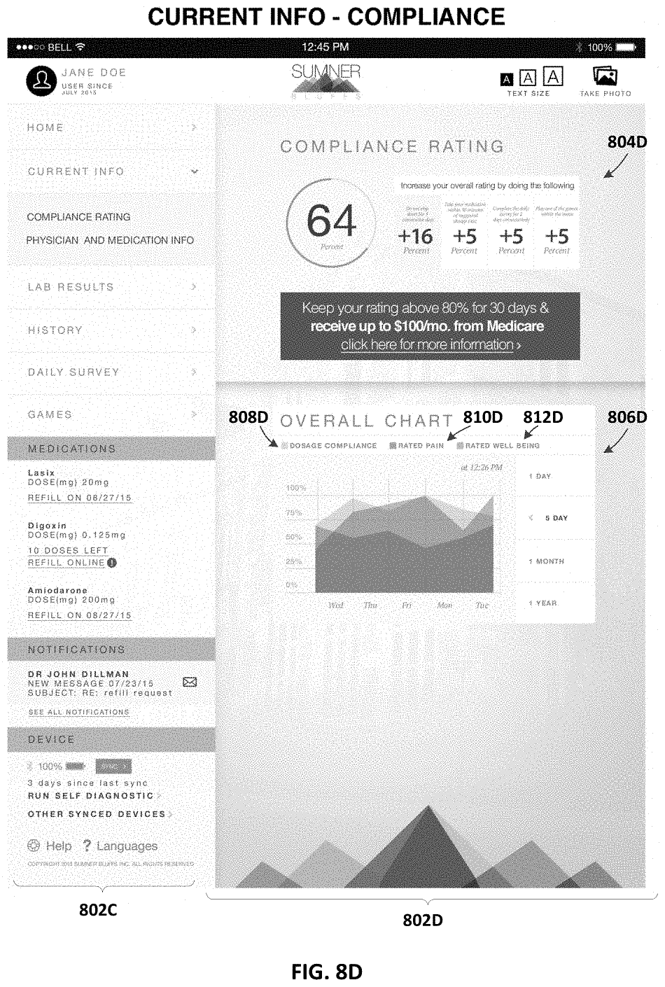

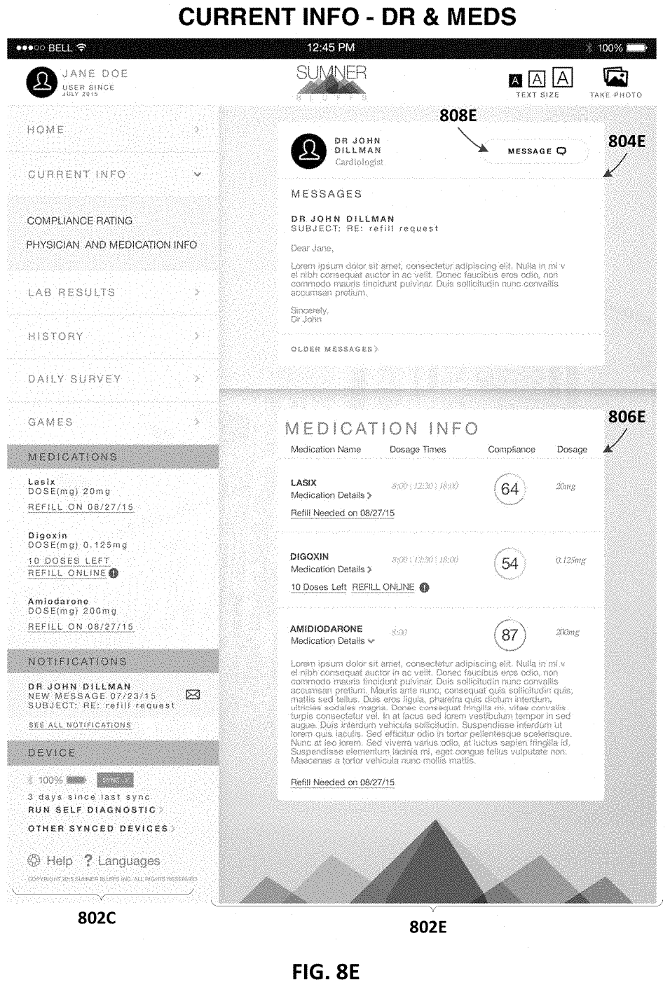

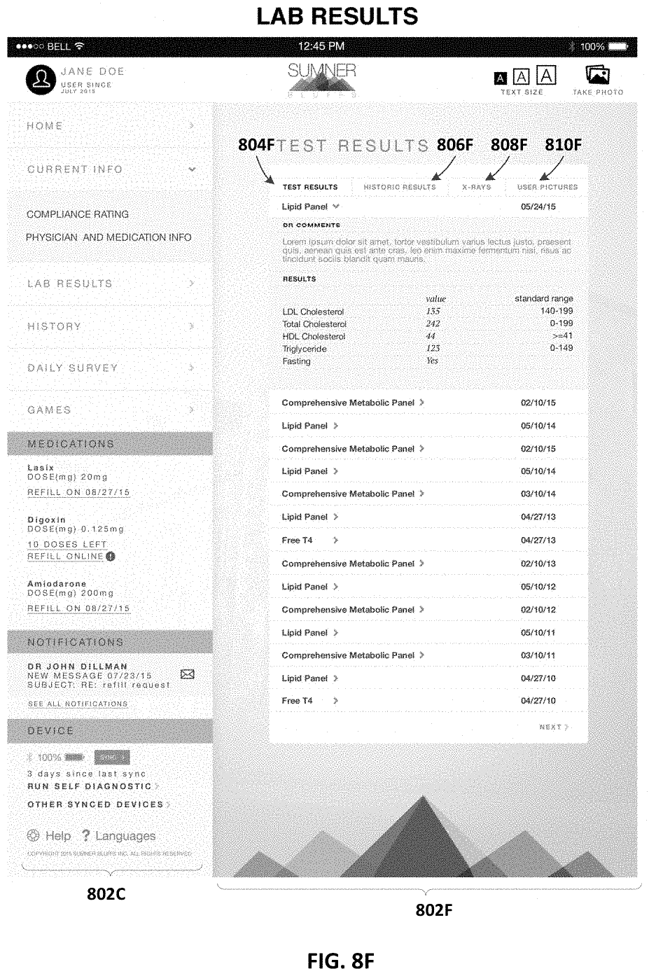

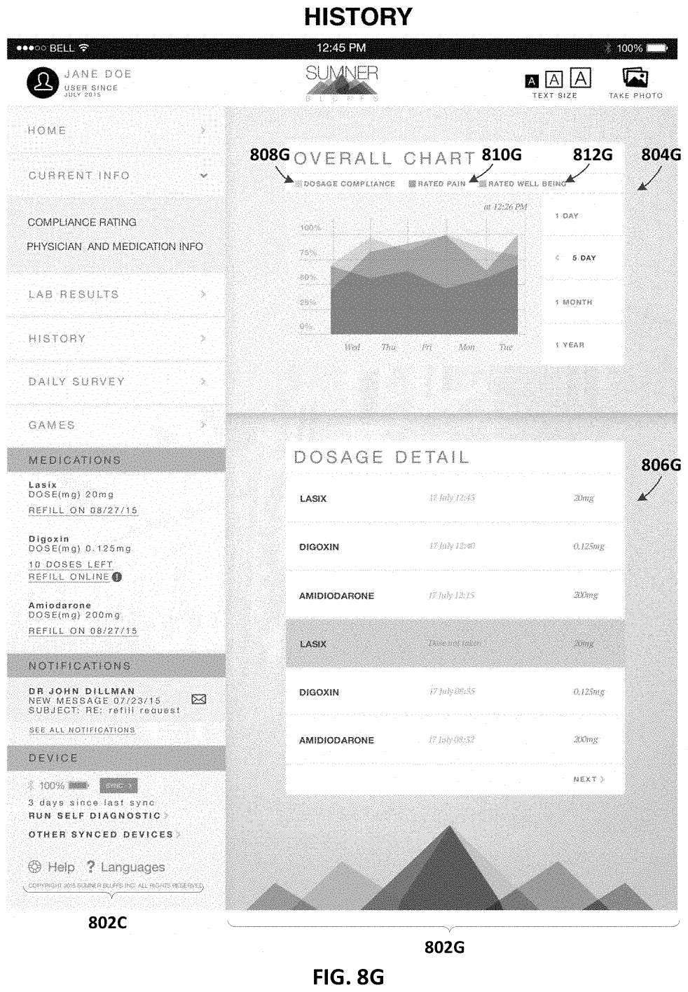

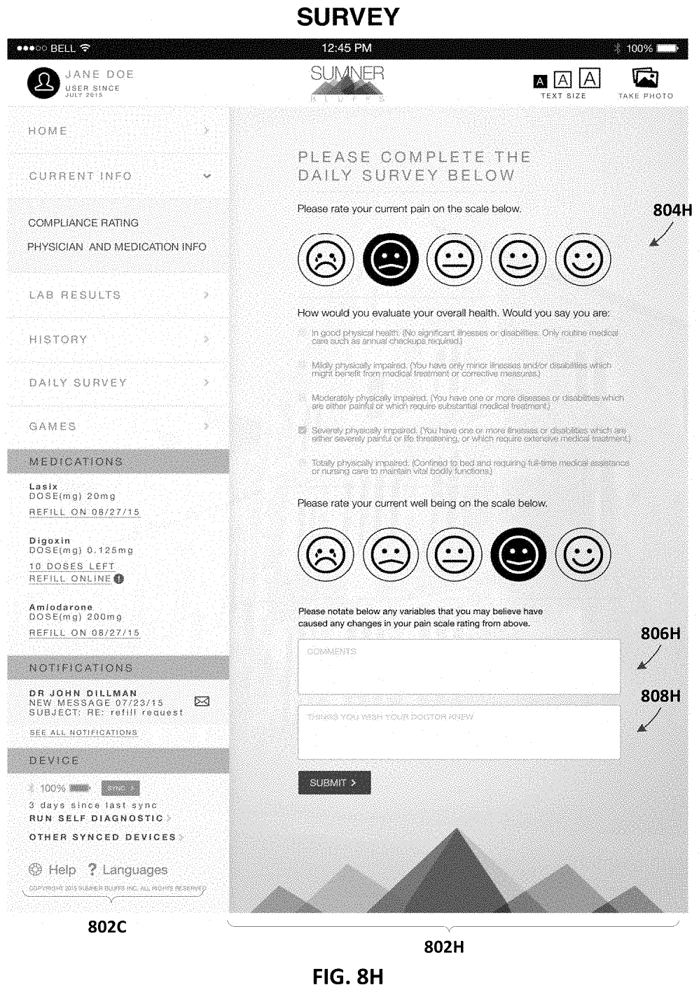

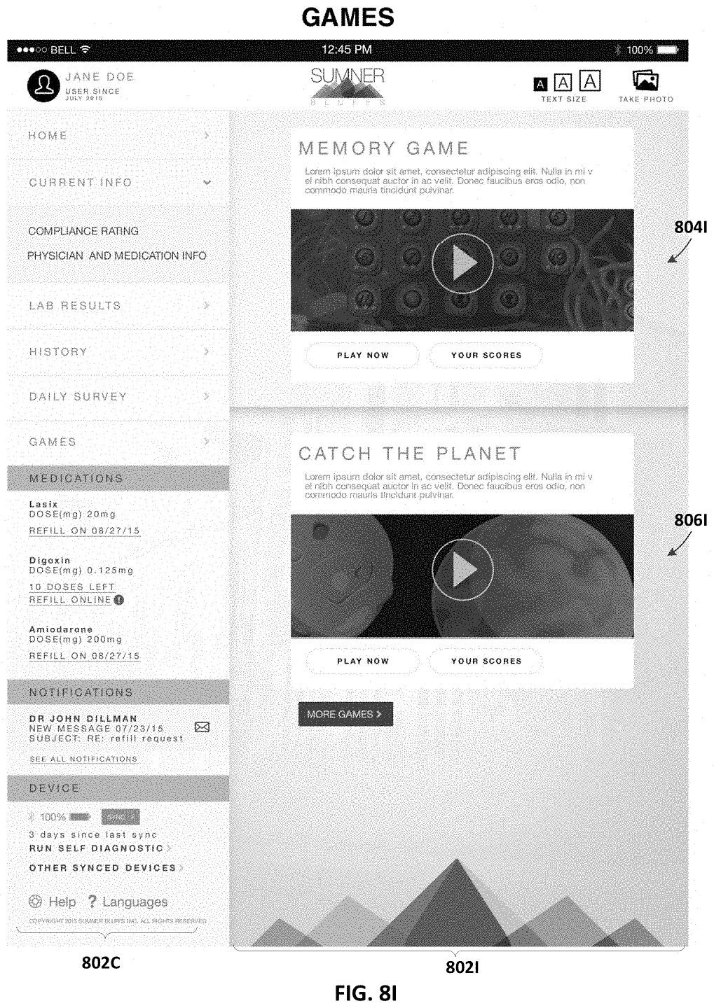

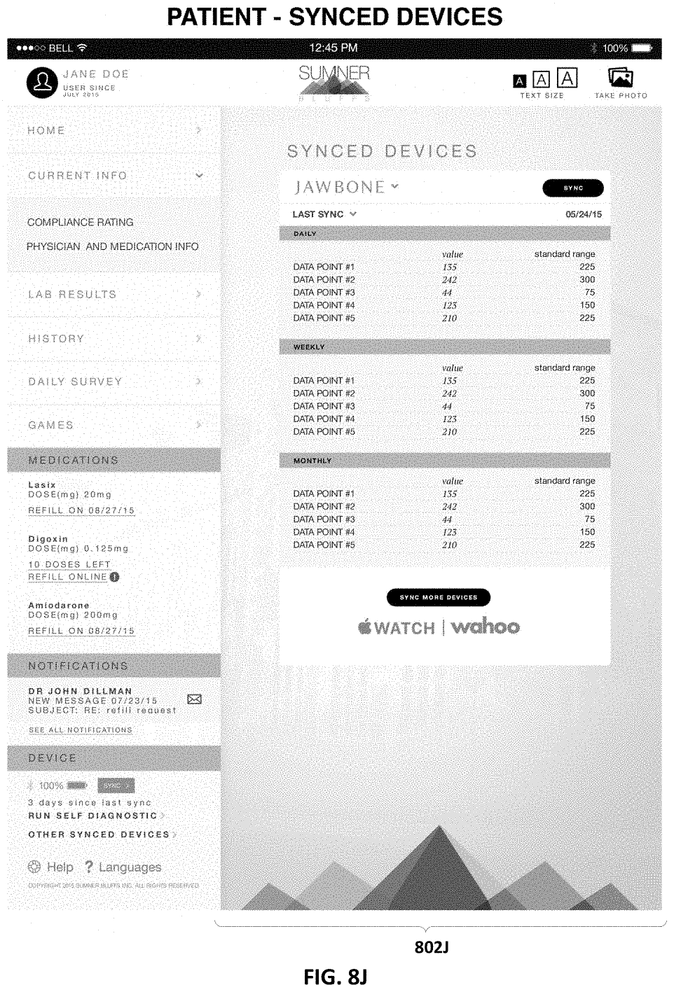

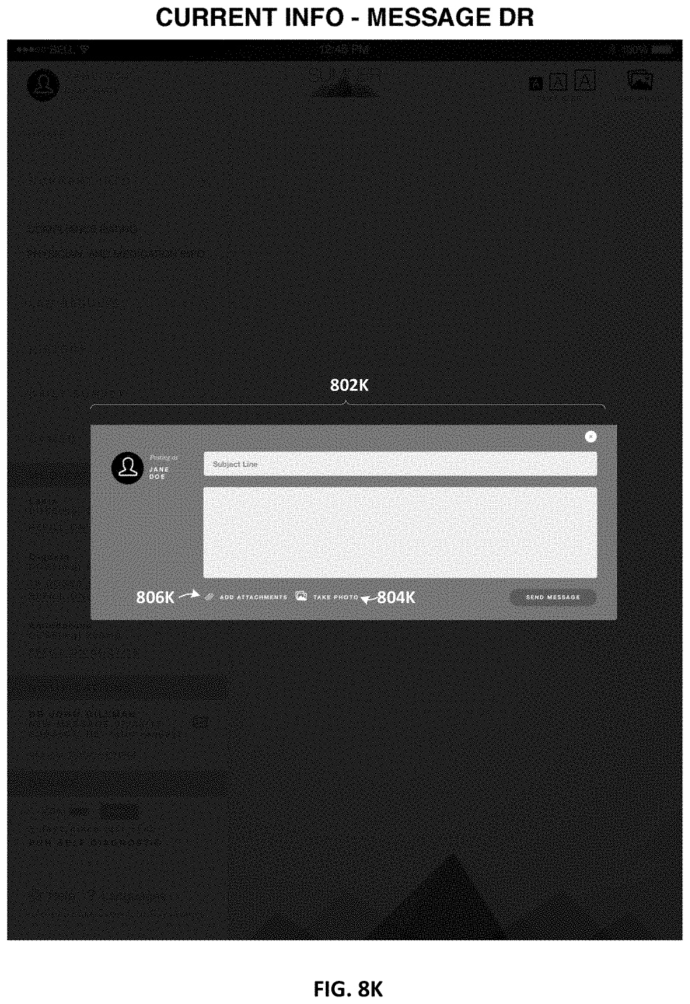

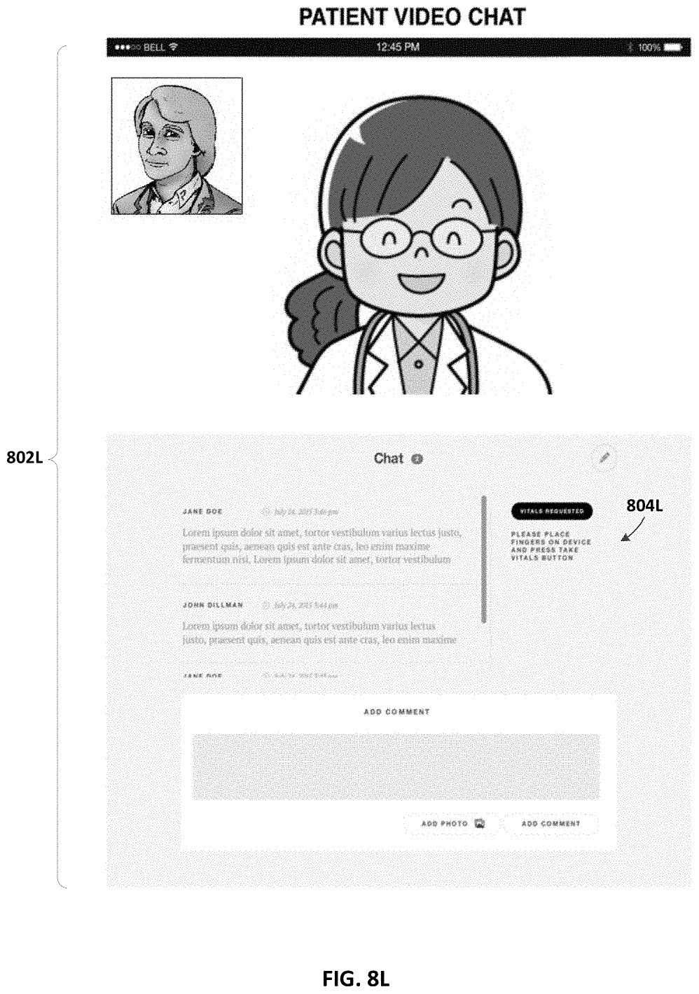

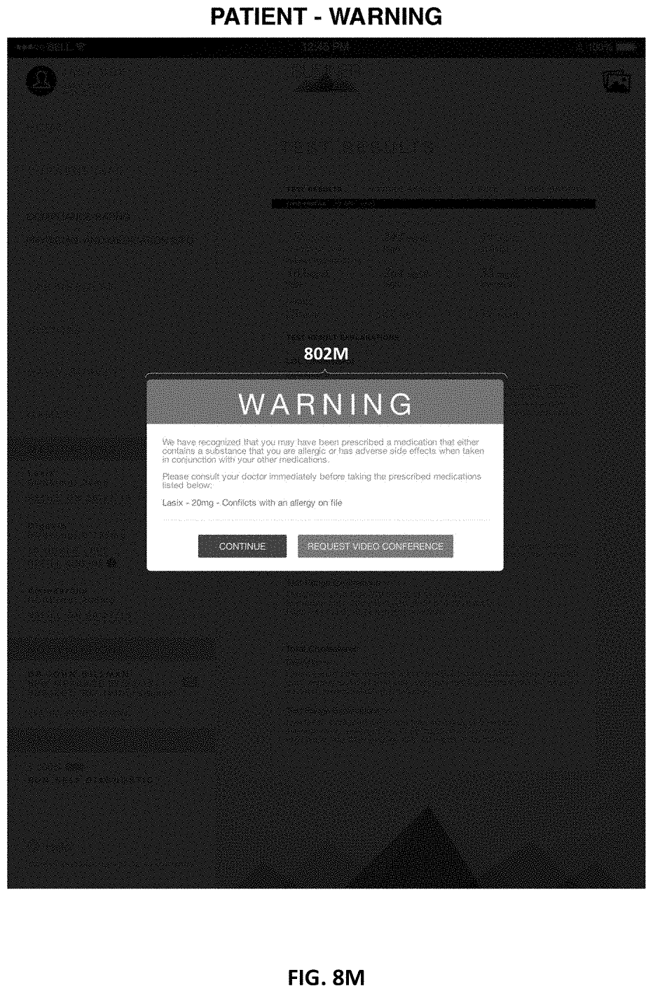

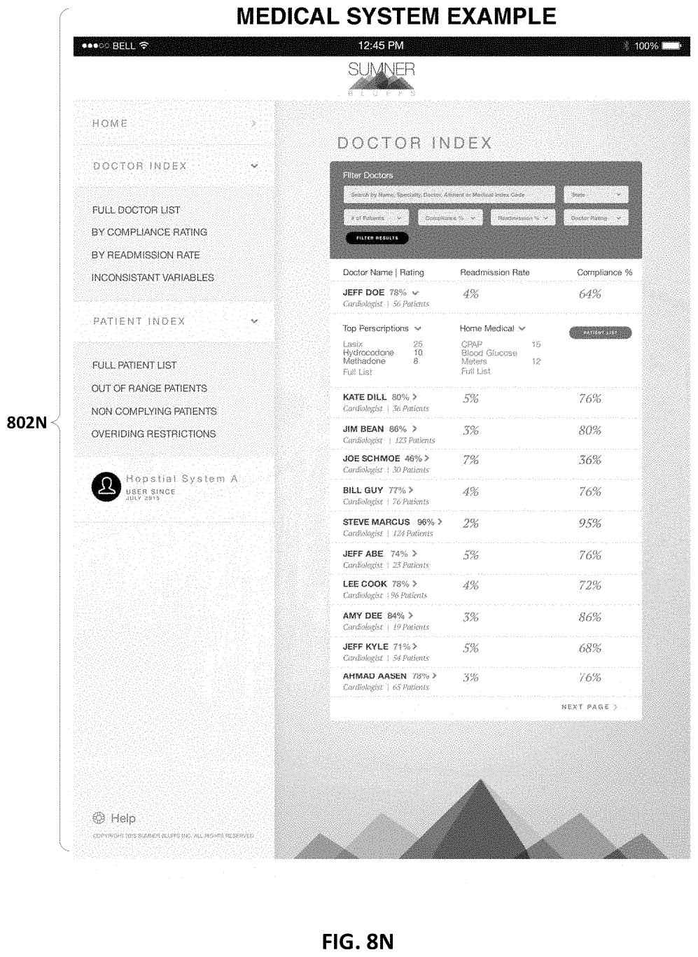

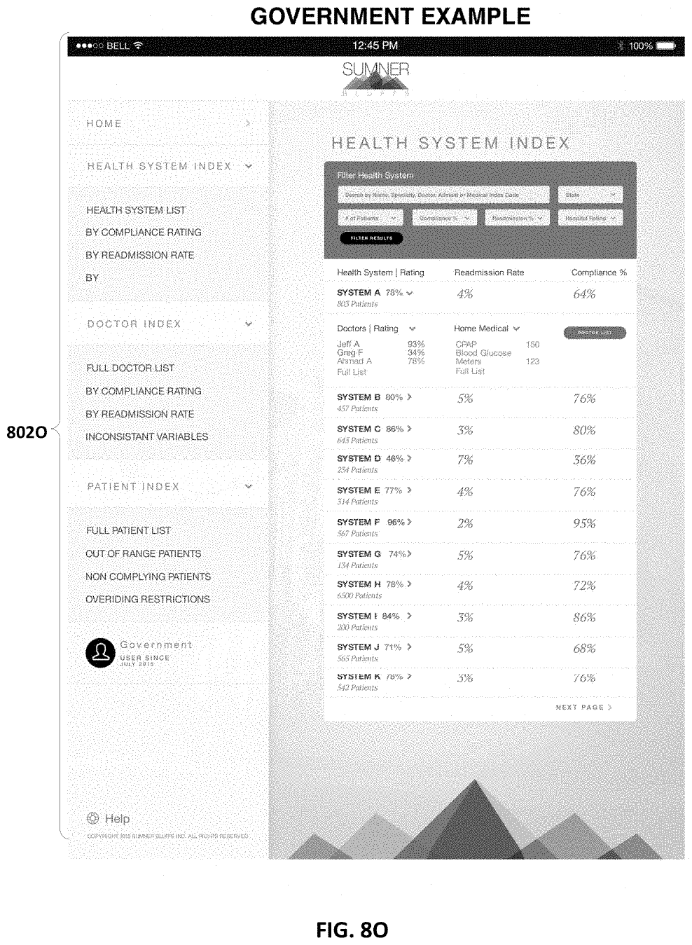

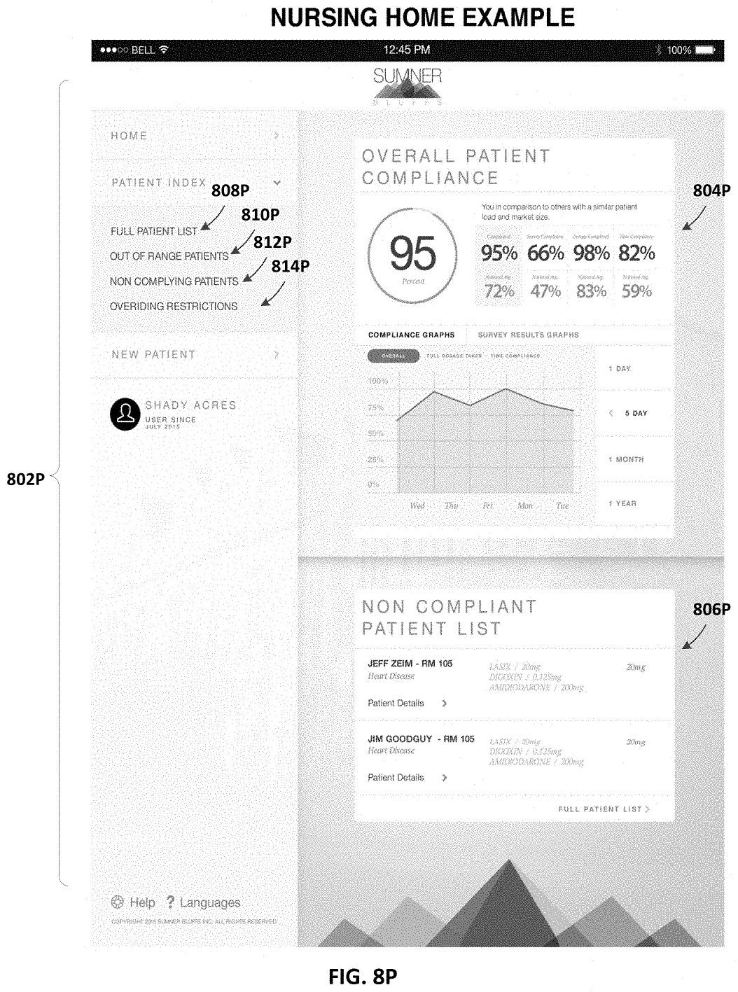

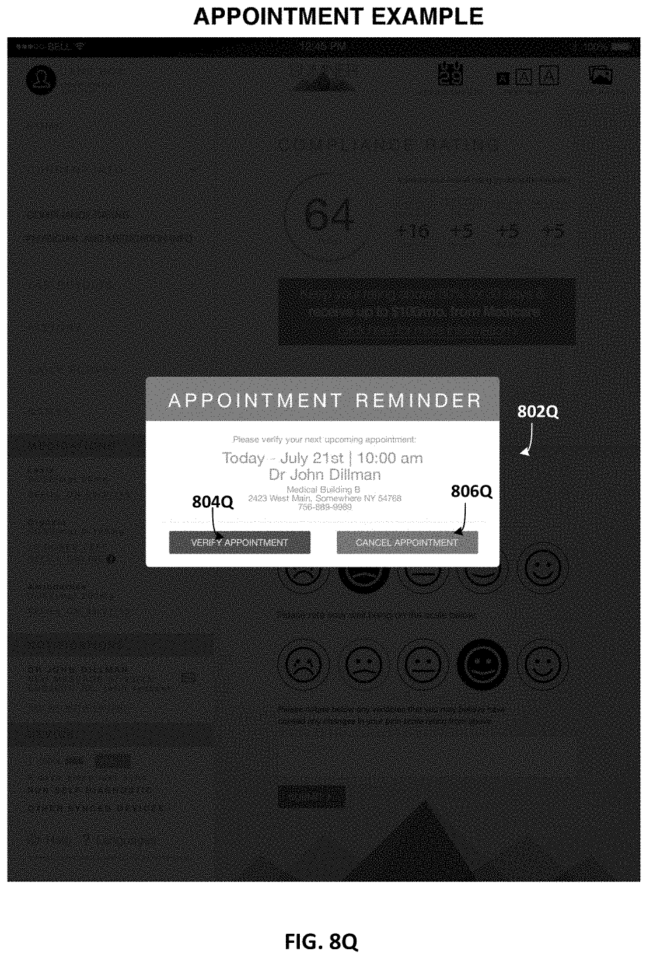

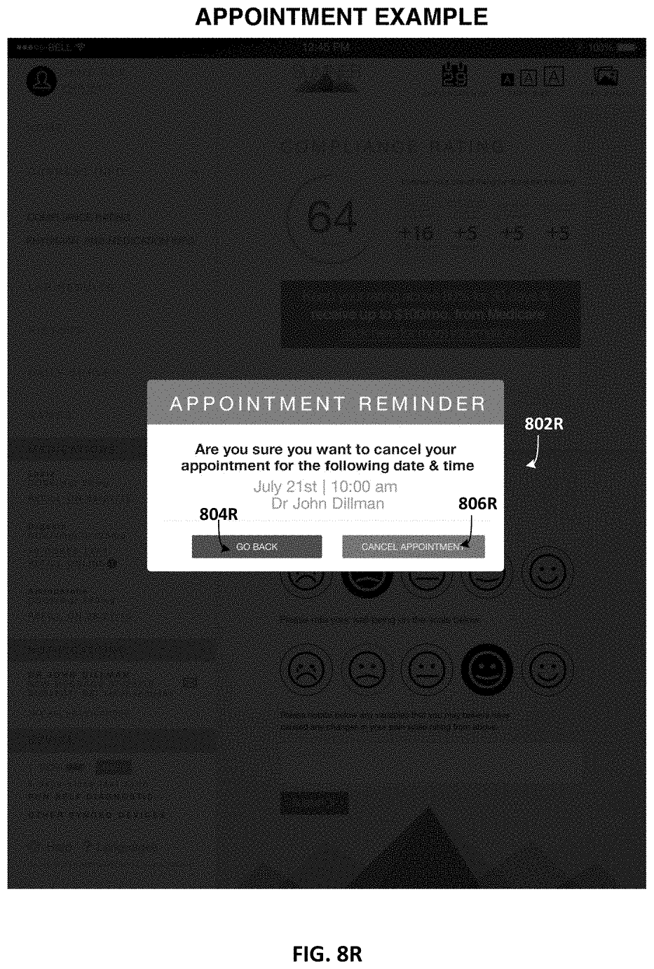

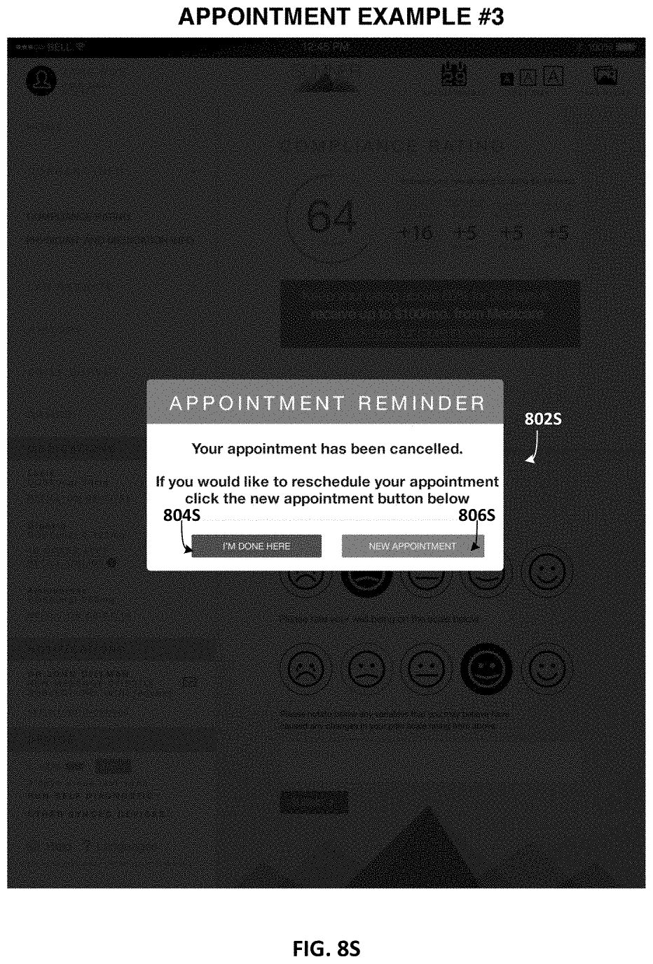

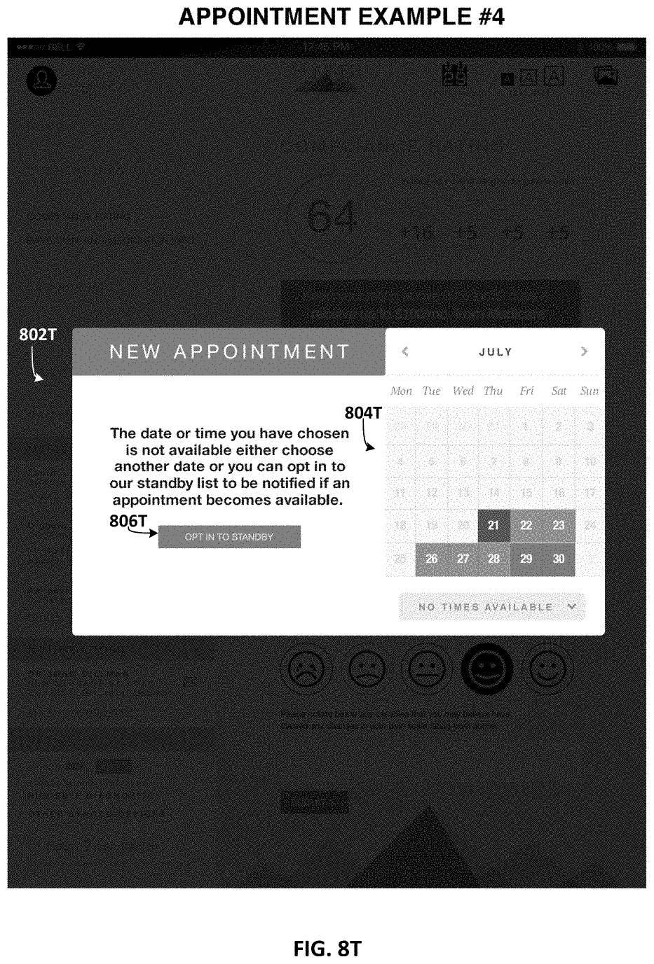

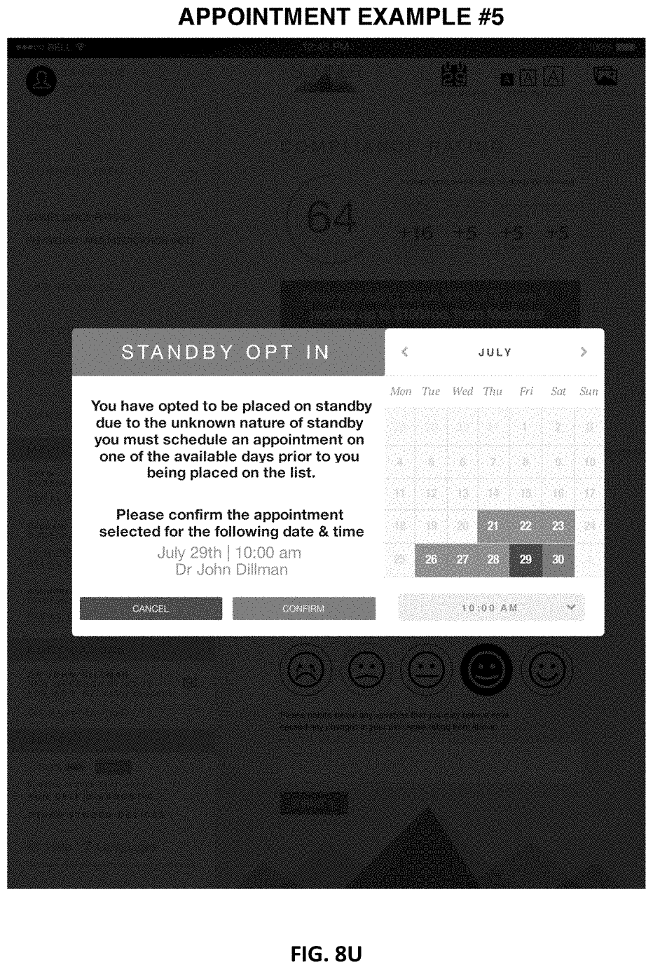

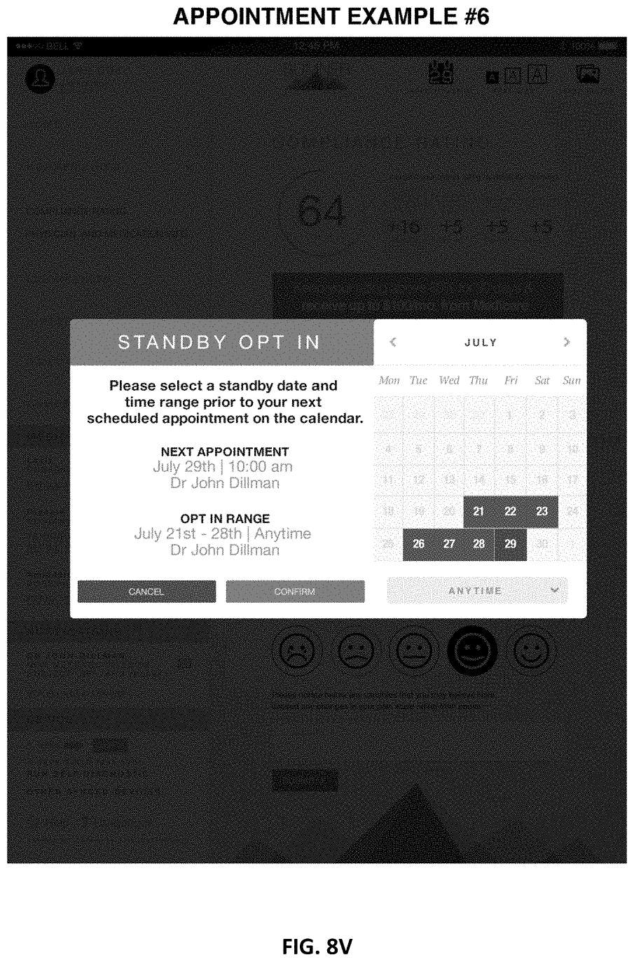

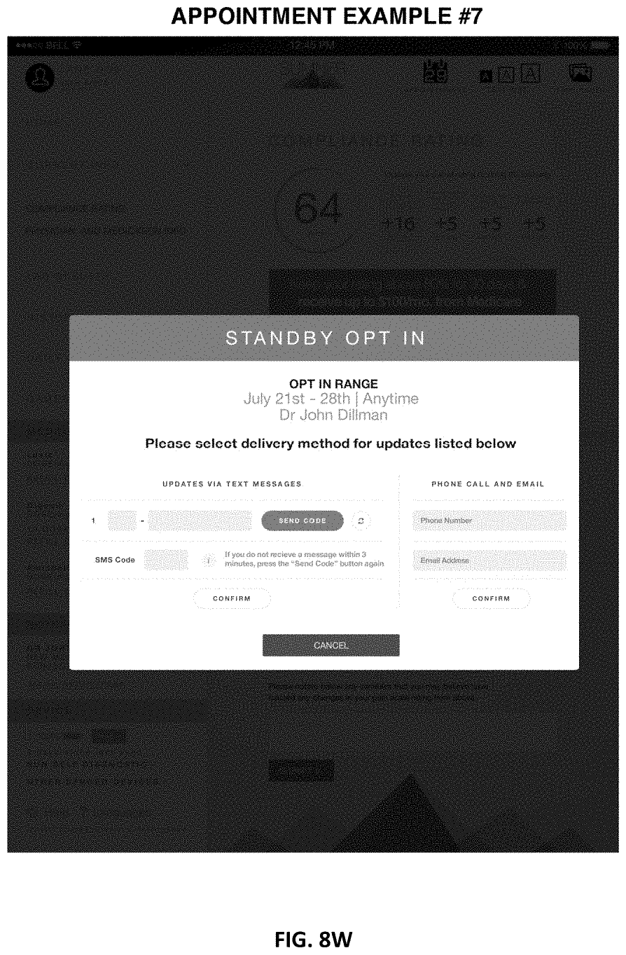

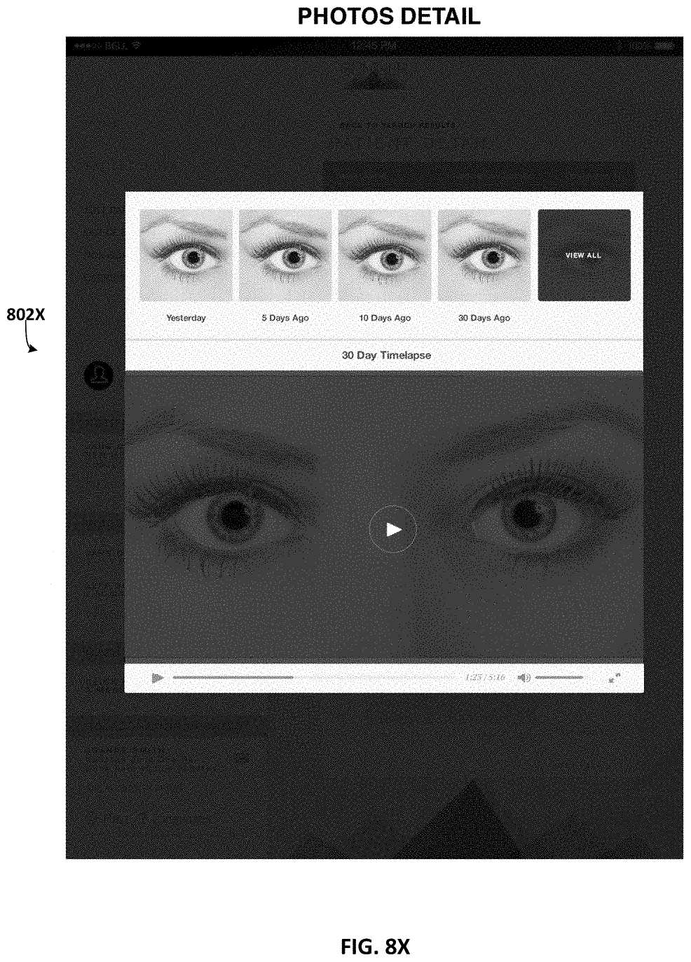

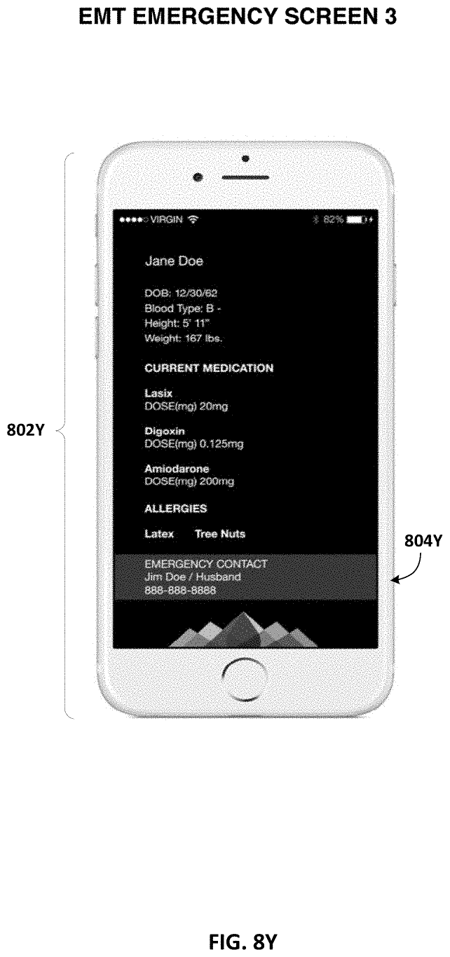

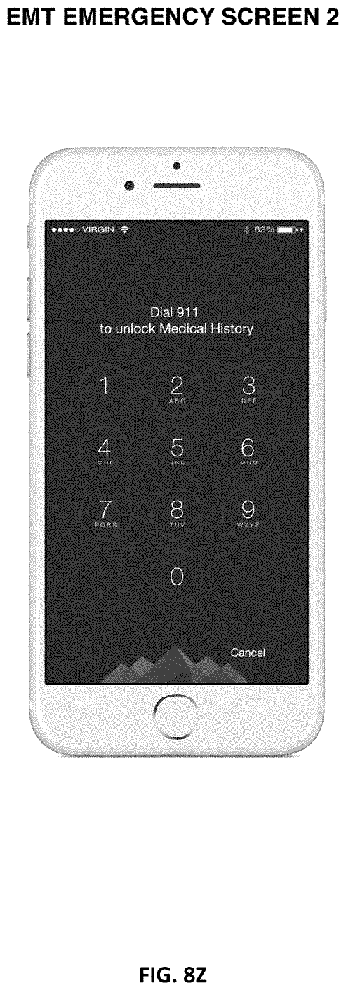

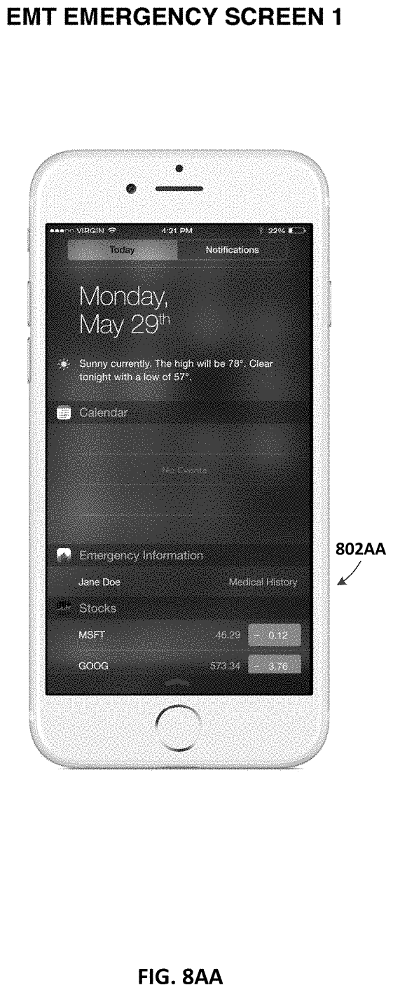

FIGS. 8A-8AA exemplary embodiments of a user interface (“UI”), which can run on a computing device (e.g., the secondary device 200 in FIG. 1), and implements the features and methods of FIG. 7. In some embodiments where a user is visually impaired or has another condition that does not allow the user to see or read the content on the page, the UI on each page can be adapted to these various conditions. For example, if a user is visually impaired, images can be replaced with text equivalents, so that an assistive screen reader can read the information aloud for the user. Furthermore, the Tab key (or equivalent on a smartphone) can be used to navigate through the various text fields; and, once a user navigates to a new text field, the text field can be read aloud for the user. The user can then use the “Enter” key (or equivalent on a smartphone) to select an object, instead of using a mouse click (or touch on a smartphone). Alternatively, or additionally, in embodiments where a user is visually impaired or has another condition that does not allow the user to see or read the content on the page, a hearing impaired call system can be utilized to read the information on the pages to the user.

FIG. 8A is an exemplary log in page 800A for a user. In the illustrated embodiments, the exemplary log in page 800A can include a sign up icon 802A that links to a user sign-up page (not shown). On the user sign-up page, a user can choose a username and a password. Additionally, a user can enter in personal and medical information. Some examples of personal and medical information can include, but are not limited to, the following: the user's sex, date of birth, height, weight, blood type, body mass index, body fat percentage, current prescriptions, and medical history. On the user sign-up page, a user can also be given the option to sync their pharmaceutical agent delivery and biometric data acquisition device 100 with their user profile using a wired or wireless (e.g., Wi-Fi™ or Bluetooth™) connection. After the user creates an account, the user can enter their user name and password in the sign in fields 804A in order to sign in. If the user enters an incorrect user name or password, they can be transferred to a bad user name page 800B, as shown in FIG. 8B. If the user needs help signing in, the user can select a help icon 806A on either the log in page 800A or the bad user name page 800B, respectively, which can direct them to a frequently asked questions (FAQs) page or other resource (e.g., a live chat) to help a user that is having any issues signing in.

In embodiments, once the user signs in, the user interface populates a user homepage 801C and a sidebar 802C, as shown in FIG. 8C. The sidebar 802C includes various tabs 804C-820C that a user can select. The various tabs 804C-820C that populate the sidebar 802C can include, but are not limited to, the following: a homepage tab 804C, a current info tab 806C, a lab results tab 809C, a history tab 810C, a daily survey tab 812C, a games tab 814C, a medications tab 816C, a notifications tab 818C and a device tab 820C. If a user navigates away from the user homepage 801C, a user can return to the homepage 801C by selecting the homepage tab 804C.

In embodiments, the user homepage 801C includes the user's compliance rating 824C. A user's compliance rating 824C can be a determination of how well a user is complying with the instructions for taking one or more pharmaceutical agents that the user has either been directed to take or is self-administering. Various compliance parameters can contribute to a user's compliance rating 824C. For example, if the user has not skipped a dose for a number of consecutive days (e.g., 5 days), then the user's compliance rating 824C may increase. If the user takes their dosage within some period of time (e.g., 30 seconds) of the suggested dosage time, then the user's compliance rating 824C may increase. If the user completes the daily survey (described below) for a number of consecutive days (e.g., 2 days), then the user's compliance rating 824C may increase. If the user plays one of the games (described below) within a period of time after taking their dosage, then the user's compliance rating 824C may increase. In embodiments, if the user is taking multiple pharmaceutical agents, the compliance rating 824C may be a composite of the compliance ratings for all of the pharmaceutical agents that a user is taking. In embodiments, the compliance rating 824C may be a compliance rating for a single pharmaceutical agent that a user is taking.

In embodiments, if the user achieves a certain compliance rating 824C, the user interface may display rewards 826C based on the user's compliance rating 824C. Example rewards 826C may include, but are not limited to, cash, gift certificates, discounts on insurance premiums and discounts on prescriptions.

In embodiments, the user homepage 801C includes an option for the user to complete a survey 828C. As stated above, in embodiments, if the user fills out the survey 828C, and how often the user fills out the survey, may contribute to the user's compliance rating 824C. The survey 828C includes questions 830C related the health and the well-being of the user. Example questions 830C may include, but are not limited to, “please rate your current pain on the scale below” and/or “please rate your well-being.” After each question 830C a number of options 832C may be displayed for the user to select. In embodiments, the survey 828C may also include a text box 834C where a user can describe other variables that may have contributed to the responses given to the questions in the survey 828C. The user may also submit additional information in the text box 834C that the user would like a third-party (e.g., the user's doctor) to know. Exemplary information that the user may enter into the text box 834C may include, for example, additional side-effects that were not described to the user previously, information about how much better the user is feeling after taking the medication, etc.

As presented above, the sidebar 802C may include a current info tab 806C. The current info tab 806C may include one or more subsidiary tabs. Examples of subsidiary tabs may include, but are not limited to, a current info compliance rating tab 807C and current info physician and medication info tab 808C.

Referring back to the sidebar 802C, after selecting the current info compliance rating tab 807C, UI may display a corresponding current info compliance rating page 802D concurrently with the sidebar 802C, as shown in FIG. 8D. The current info compliance rating page 802D may include the user's compliance rating 804D and an overall compliance rating chart 806D. The overall compliance rating chart 806D may include selectable icons 808D, 810D, 812D that, when selected, can show a respective chart for dosage compliance 808D, rated pain 810D and rated well-being 812D. A dosage compliance chart 808D illustrates in a chart form the user's dosage compliance over a period of time. The rated pain chart 810D illustrates in a chart form the user's pain over a period of time, as determined by the responses to the survey 828C. The rated well-being chart 812D displays a chart of the user's well-being over a period of time, as determined by the responses to the survey 828C. For each respective chart 808D, 810D, 812D, different time periods can be selected to display (e.g., 1 day, 2 days, 3 days, 4 days, 5 days, 1 week, 1 month, and 1 year or any desired time period).

Referring back to the sidebar 802C, after selecting the current info physician and medication info tab 808C, the UI can display a corresponding current info physician and medication page 802E concurrently with the sidebar 802C, as illustrated in FIG. 8E. In exemplary embodiments, the current info physician and medication page 802E can display messages from third parties 804E. For example, a message can be a note from a doctor instructing the user to increase or decrease the dosage of a pharmaceutical agent per delivery time period or increase or decrease the frequency of administration etc. As another example, a message can be a message from a doctor instructing the user to discontinue taking the pharmaceutical agent. In addition to reading a message from a third party under the messages or notifications from third parties 804E, a user can also respond to the message or confirm receipt of the message, as discussed below. In some embodiments, messages from third parties 804E can also be displayed after a user selects the notifications tab 818C from the sidebar 802C.

In addition to displaying messages from third parties 804E, current info physician and medication page 802E can display the user's medication info 806E. The user's medication info 806E can include pharmaceutical agents that the user is currently taking and pharmaceutical agents that the user used to take in addition to the monitored agent or pharmaceutical. For each pharmaceutical agent, the pharmaceutical agent's name, the dosage times, the compliance rating and the dosage can be displayed in the user's medication info 806E. Additionally, any side effects of the pharmaceutical agent can be listed, along with any pharmaceutical agents that should be avoided when taking the pharmaceutical agent.

User's medication info 806E can also be displayed after a user selects the medications tab 816C from the sidebar 802C. In exemplary embodiments, the medications tab 816C can include a listing of the pharmaceutical agents that a user is taking, the dosage of each medication, the date the medication was prescribed, when a refill is needed, how many doses are left before a refill is needed and whether the user can obtain a refill online.

Referring back to the sidebar 802C, after selecting the lab results tab 809C, the UI can display a corresponding lab results page 802F concurrently with the sidebar 802C, as illustrated in FIG. 8F. The test results page 802F can link to the user's medical records. As such, the test results page 802F can include, but is not limited to, the user's most recent test results 804F, the user's historic test results 806F, the user's x-rays 808F and the user's pictures 810F. The user's most recent test results 804F and the user's historic test results 806F can include, but is not limited to, the date the test was administered to the user, the results on the test (e.g., triglyceride value and LDL, HDL and total cholesterol for a lipid test) and whether the doctor had any comments or recommendations. The user's x-rays 808F and the user's pictures 810F can also include, but is not limited to, the date the x-ray was taken and the date the picture was taken, respectively. As is discussed throughout the application, pictures of the user's eyes, retinas, facial features, wound site, surgical site and other features of the user can be used to determine how a user is reacting to an administered pharmaceutical agent.

Referring back to the sidebar 802C, after selecting the history tab 810C, the UI can display a corresponding history page 802G concurrently with the sidebar 802C, as illustrated in FIG. 8G. The history page 802G can include an overall chart 804G and a dosage detail chart 806G. The dosage detail chart 806G can include all the pharmaceutical agents that a user is taking or has taken. Furthermore, when selecting a pharmaceutical agent in the dosage detail chart 806G, the overall chart 804G can display information pertaining to the selected pharmaceutical agent. In some embodiments, the overall chart 804G can be similar to the overall compliance rating chart 806D discussed above, and can include selectable icons 808G, 810G, 812G that, when selected, can illustrate a corresponding chart for dosage compliance 808G, rated pain 810G and rated well-being 812G for the selected pharmaceutical agent in the dosage detail chart 806G. Moreover, for each chart 808G, 810G, 812G, different time periods can be selected to display (e.g., 1 day, 2 days, 3 days, 4 days, 5 days, 1 week, 10 days, 1 month, and 1 year or any desired period of time).

Referring back to the sidebar 802C, after selecting the daily survey tab 812C, the UI can display a corresponding survey page 802H concurrently with the sidebar 802C, as illustrated in FIG. 8H. The survey page 802H can include questions 804H that can be administered to the user, which the user can then respond to. For example, questions 804H can include, but are not limited to, “how would you evaluate your overall health?” and then display a series of options such as a numbers scale or similar, for example where 1 is the worst and 10 is the best rating. In exemplary embodiments, a first text box 806H can be included in the survey page 802H where a user can describe other variables that may have contributed to the responses given to the questions 804H. Furthermore, a second text box 808F can be included in the survey page 802H wherein a user can include information that they would like shared with his/her doctor. For example, the user may include information about additional side-effects that were not described to the user previously but that the user is experiencing. In addition, the user may include information about improvements in his/her health the user experienced after taking the medication. In certain embodiments, the responses of the user can be utilized to assist in determining a response rating.

Referring back to the sidebar 802C, after selecting the games tab 814C, the UI can display a corresponding games page 802I concurrently with the sidebar 802C, as shown in FIG. 8I. The term “games” referred to herein can be a subset of tests that are discussed in FIG. 7 above. The tests provided can be, but are not limited to, medical well-being tests that correlate with improved health of the user or demonstrate no change or failing health of the user. The games page 802I can include different games that correlate, in exemplary embodiments, to the type of medication that a user is taking. For example, if the user is taking a pharmaceutical agent that is supposed to treat some of the symptoms of Alzheimer's, then a memory game 804I can be displayed on the games page 802I. As another example, if the user is taking a pharmaceutical agent that is supposed to increase a user's hand-eye coordination, then a hand-eye coordination game 806I can be displayed on the games page 802I. The games 804I, 806I that are displayed on the games page 802I can be selected and played by a user. The responses to the games 804I, 806I can be used to determine a response rating. Any other self-administering test is contemplated herein.

Referring back to the sidebar 802C, in other embodiments, under the device tab 820C, characteristics about the device that is running the user interface can be illustrated. For example, the battery life left on the device can be displayed. Furthermore, in certain embodiments, a synced devices page 802J can be displayed after the device tab 820C is selected, as illustrated in FIG. 8J. The synced devices page 802J can include, but is not limited to, the various devices that are synced with the user interface and for example, how long it has been since the devices have been synced. Examples of devices that can be synced to the user interface include, but are not limited to, the pharmaceutical delivery and biometric monitor device 100 discussed above, the Jawbone™, the Apple Watch™ and Wahoo fitness™ device or any similar device. In certain embodiments, data from synced devices can be used to determine a compliance rating or a response rating or both. For example, information from the pharmaceutical delivery and biometric monitor device 100 can be synced with the user interface to determine a compliance rating. As another example, a Jawbone™ can be synced to the user interface so that data from the Jawbone™ can be uploaded to the user interface to determine activity level of a user. Here, the term Jawbone™ refers to the wearable wristband that tracks steps, sleet, exercise, et al. Information from a Jawbone™ can be advantageous in determining a user's response to a pharmaceutical agent. For example, even if the user of the pharmaceutical agent doesn't seem to be feeling better, if they take significantly more steps throughout a day after taking the pharmaceutical agent than they did before taking the pharmaceutical agent, that may be an indication that they could be feeling a little better.

As illustrated in the sidebar 802C, a self-diagnostic test can also be run by selecting the appropriate icon under the device tab 820C. The self-diagnostic test can determine whether the most recent version of the user interface is running on the device and determine whether there is a connection established (e.g., Bluetooth™ connection) established between the user device and the synced device. The language displayed in the user interface can also be changed by selecting a “Languages” icon under the device tab 820B. Example languages can include, but are not limited to, German, French, English (as shown), Italian, Spanish, Mandarin and Hindu.

As presented above, if a user wants to respond to a message from a third party 804E (shown in FIG. 8E), a message page 802K (shown in FIG. 8K) can be displayed after selecting a message icon 808E (shown in FIG. 8E). Referring to FIG. 8K, the user can type their message in the text box in the message page 802K and send the message to the person who originally sent the message 804E. The user can also include a photo by taking a photo using a take photo icon 804K or an attachment by selecting an attachment icon 806K. In some embodiments, the message page 802K can be displayed after a user selects a messaging tab (not shown) from the home page 801C, if the user would like to send a message to a third-party without having to respond to a message 804E.

In some embodiments, a video chat page 802L, as shown in FIG. 8L, can be used to respond to a message 804E. In some other embodiments, the video chat page 802L can be displayed after selecting a video chat tab (not shown). In some embodiments, a third-party can request a video chat with the user in a message 804E and the user can accept the video chat request, which will enable the video chat page 802L. When a user is chatting with the third-party, the user can be directed to take their vitals and upload them, using the vital icon 804L on the video chat page 802L.

In some embodiments, a warning page 802M can be displayed in the user interface, as shown in FIG. 8M. As an example, the warning page 802M can be displayed in the user interface when a user has been directed to take a pharmaceutical agent that contains an allergic substance to the user. Or, as another example, the warning page 802M can be displayed when a user has been directed to take a pharmaceutical agent that may have an adverse side effect when taken in conjunction with other pharmaceutical agents that the user is directed to take. In some embodiments, as shown in the warning page 802M, a description of the pharmaceutical agent that resulted in the warning can be shown in the warning page 802M. In some embodiments, the warning page 802M may include a button that allows a user to request a video conference with the third-party that directed the user to take the pharmaceutical agent.

In other embodiments, a doctor index page 802N can be displayed in the user interface, as shown in FIG. 8N. The doctor index page 802N can be displayed in the user interface after a user selects a doctor index tab (not shown). In some embodiments, the doctor index page 802N can be populated based one or more of the following: the doctor's name, the title and type of practice of the doctor, the hospital the doctor is affiliated with, the office address of the doctor, the doctor's phone number, the doctor's email, users' reviews of the doctor, how often the patients of the doctor get readmitted (e.g., readmission rate), the compliance percentage of the patients for the doctor, the location of the doctor relative to the user, and whether the doctor is in the user's insurance network. This list, however, is not meant to be exhaustive and, as a result, other factors may determine how the doctor index page 802N is populated. In some embodiments, the doctor index page 802N is only available to a hospital's administration for determining which doctors have the highest readmission and compliance rating.

In embodiments, a health system index page 802O can be displayed in the user interface, as shown in FIG. 8O. In embodiments, the health system index page 802O can be displayed in the user interface after a user selects a health system tab (not illustrated). In embodiments, the health system index page 802O can display statistics about different hospitals (referred to as System A, B, C, etc.) For example, the health system index page 802O can display a readmission rate for a hospital. In embodiments, the readmission rate can be displayed alongside the compliance rate for users. As a result, a user can make a better determination as to whether a high readmission rate is solely a result of the hospital's care and policies or whether it is partially due to a user not complying with a dosing regimen.

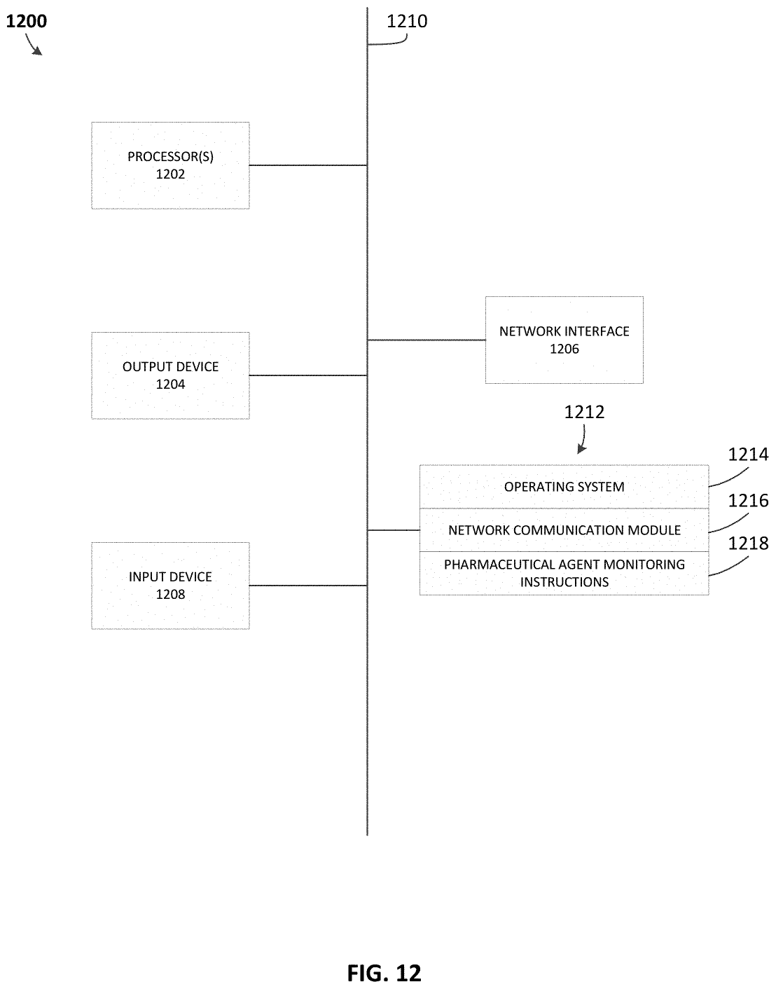

In embodiments, the health system index page 802O may also track International Statistical Classification of Disease (ICD) codes for a hospital. In embodiments, the ICD codes can be used by a user to determine whether a hospital has performed a certain procedure before and the frequency that the hospital performs the procedure. In embodiments, the ICD codes may also be used to track fraud, as described in FIG. 12 below.

In embodiments, if a hospital is selected by a user, the user may also see a list of the doctors that practice at the hospital. After which, a doctor can be selected to see additional information about the doctor (e.g., specialty, whether the doctor is in a user's insurance network, compliance rating of the doctor's patients, etc.) In some embodiments, the health system index page 802O may only be available to government users, having the appropriate access, for determining which hospitals have the best readmission and compliance ratings.

In exemplary embodiments, a nursing home page 802P can be displayed in the user interface, as shown in FIG. 8P. In some embodiments, the nursing home page 802P can be displayed in the user interface after a user selects a nursing home tab (not shown). In some embodiments, the nursing home page 802P can display an overall user compliance rating 804P (displayed as “overall patient compliance”) for users that reside at the nursing home. In addition, the nursing home page 802P can also display a list of non-compliant user (displayed as “non-compliant patient list”) 806P. In some embodiments, the list of non-compliant users 806P can be displayed after selecting a non-complying patients tab 812P. Other tabs in the nursing home page 802P can include, but are not limited to, a full patient list tab 808P, an out of range patients tab 810P which includes users that are located off-premises of the nursing home, and an overriding restrictions tab 814P which includes users that are overriding any restrictions placed on the pharmaceutical agent they were directed to take.

Referring back to the homepage 801C, after selecting the appointments icon 836C, the UI can display upcoming appointments for the user. In exemplary embodiments, the upcoming appointments correspond to appointments (either in person or via video conference) with a third-party that has directed the user to take a pharmaceutical agent. In exemplary embodiments, appointment reminders 802Q can be given for upcoming appointments, as shown in FIG. 8Q. In embodiments, the appointment reminder 802Q may display icons for either verifying the appointment 804Q or canceling the appointment 806Q. If the user selects the icon to cancel the appointment 806Q, the UI can display an appointment reminder cancel screen 802R, as shown in FIG. 8R.

On the appointment reminder cancel screen 802R, a user can choose to go back to the previous page by selecting a go back icon (or equivalent icon) 804R if the cancel icon 806Q was selected unintentionally or the user changed his/her mind. The appointment reminder cancel screen 802Q may also display a cancel appointment icon 806R to ensure the user wants to cancel his/her appointment and the selecting of the cancel appointment icon 806Q was not done unintentionally. If a user chooses to cancel an appointment, a rescheduling message 802S can be displayed, as shown in FIG. 8S. The rescheduling message 802S includes an “I'm done here”, “Action Complete” (or other equivalent phrase) icon 804S, which a user can select if the user does not wish to reschedule their appointment. The rescheduling message 802S can also include a new appointment icon 806S which, after a user selects, can able the user to create a new appointment page 802T to be displayed, as illustrated in FIG. 8T. The user can then select, using the calendar 804T displayed in the new appointment page 802T, a time and date for the new appointment.

If an appointment time is not available, a user can opt in to standby mode by selecting an “opt in” to standby icon 806T. By selecting the “opt in” to standby icon 806T, if the person that has the appointment time selected by the user cancels or reschedules their appointment, the user can then be notified that the appointment time has become available. In embodiments, two different types of standby mode are available. The first type, as shown in FIG. 8U, can be where a specific date and time are selected by the user. The second type, as shown in FIG. 8V, can be where a time range is selected by the user. Thus, if any appointment time becomes available in the time range, the user will be notified.

When choosing to opt in to standby mode, a user can select the type of delivery method for updates to standby appointment times, as illustrated in FIG. 8W. Exemplary types of delivery methods include, but are not limited to, a Short Message Service (“SMS”) text message, a phone call, a pop-up message on the user's electronic device or an email. In some embodiments, if two users opt in to standby mode and select the same time and date (or time period), the UI can notify the users of the available time and date for the appointment and the first user to accept the appointment time and date can be the user that is scheduled for the appointment time and date. In other embodiments, if two users opt in to standby mode and select the same time and date (or time period), the first user that opted in to standby mode and selected the appointment time and date first will be given priority to accept the appointment time and date (e.g. on a first come first serve basis), if the appointment time and date becomes available. In accordance with these embodiments, however, the first user given priority may only have a specified amount of time to respond (e.g., 30 minutes, 1 hour, 5 hours, 12 hours, 1 day, etc.).

In other embodiments, a user can choose a primary appointment time that the user opts in to using standby mode while simultaneously choosing a secondary appointment time that is open. Thus, if the primary appointment time never becomes available, the user still has the secondary appointment time to meet with the third-party.

In some embodiments, when scheduling an appointment, a user can upload photo details 802X, as illustrated in FIG. 8X, that a third-party may review before the appointment. The photo details 802X can include, but are not limited to, a time lapse of the user's eyes, retina, facial features, or other examples. The time lapse can be for a period of, e.g., 1 hour, 6 hours, 12 hours, 1 day, 5 days, 10 days, 30 days, etc. In some embodiments, after seeing the photo details 802X, the third-party may choose to see the user before the scheduled appointment if the third-party recognizes something is wrong with the user's photos that needs to be treated immediately. Furthermore, in some embodiments, after seeing the photo details 802X, the third-party may determine that only a video conference may be necessary, if the user's photos demonstrate that an in-person meeting is not necessary.

In exemplary embodiments, an Emergency Medical Technician (EMT) emergency page 802Y can be displayed in the user interface, as shown in FIG. 8Y. In some embodiments, the EMT emergency page 802Y can be displayed in the user interface after an EMT button (not shown) is selected. In some embodiments, the EMT button can be displayed on the device's locked home screen. As a result, someone (such as a medical professional) without the passcode to access the device's regular functions can still access the EMT emergency page 802Y. The EMT emergency page 802Y may include emergency contact 804Y, so that the medical professional or other person accessing the phone of the user can contact the emergency contact of the user. In some embodiments, the EMT emergency page 802Y can be displayed after typing 911 in to the device and connecting the device to a 911 operator, as shown in FIG. 8Z. This can be a way to access the EMT emergency page 802X without having the passcode to the device running the user interface. In certain embodiments where the device is unlocked, an EMT emergency page 802X can be accessed by including a link 802AA to the EMT emergency page 802Y in the same screen that calendar and stock notifications are displayed, as illustrated in FIG. 8AA. The EMT emergency page 802Y can include information about a user's date of birth (DOB), blood type, height, weight, current medications and allergies. This list, however, is not meant to be exhaustive and, as a result, other information about the user may be included in the EMT emergency as permissible page 802Y.

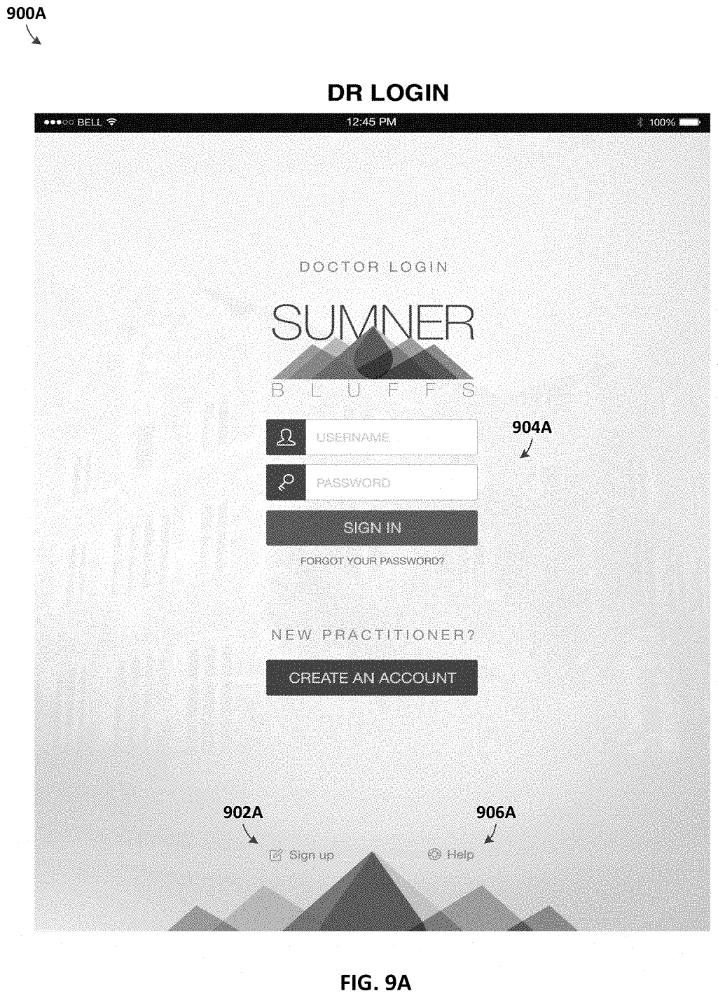

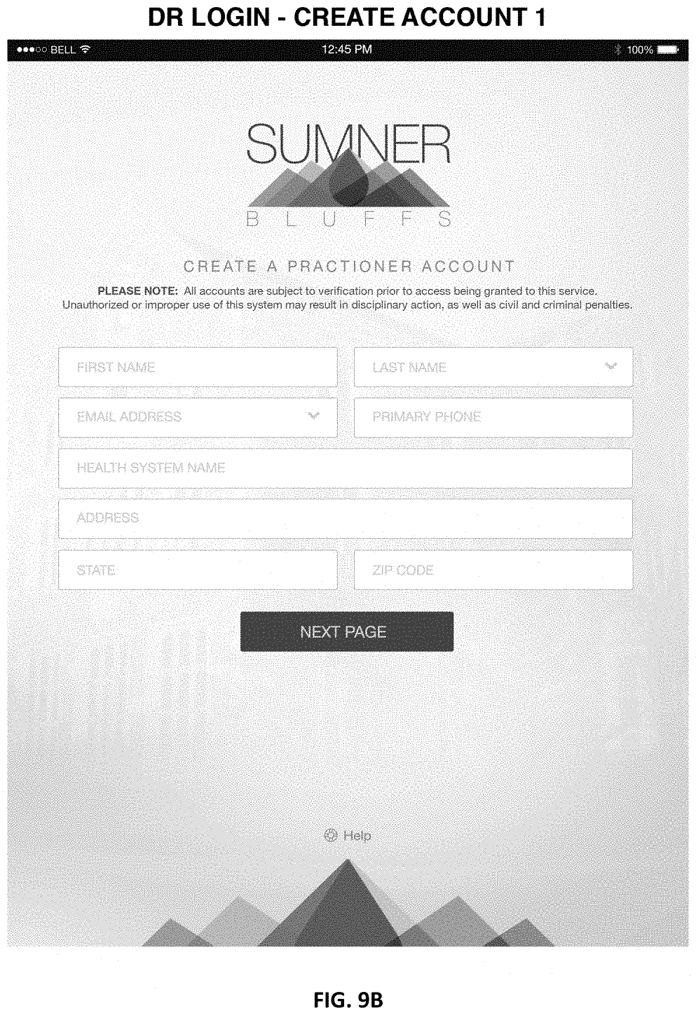

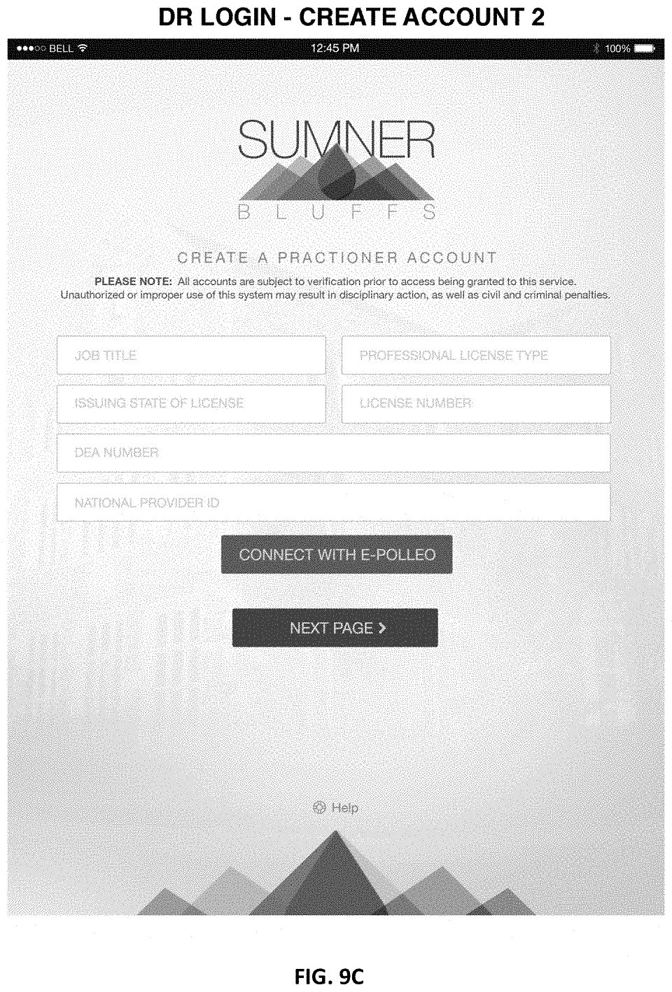

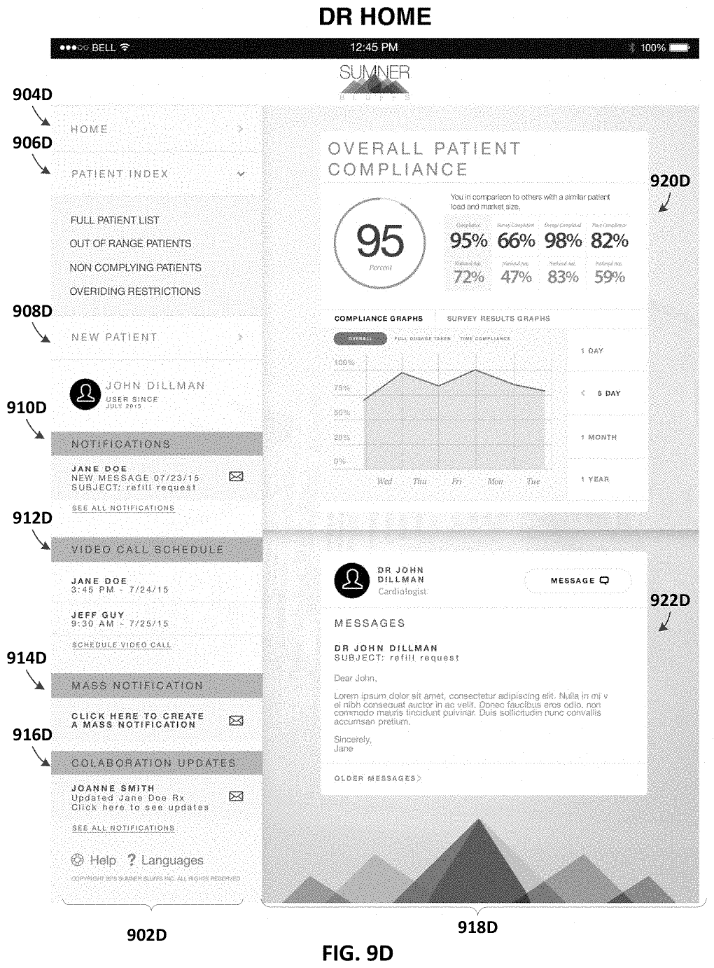

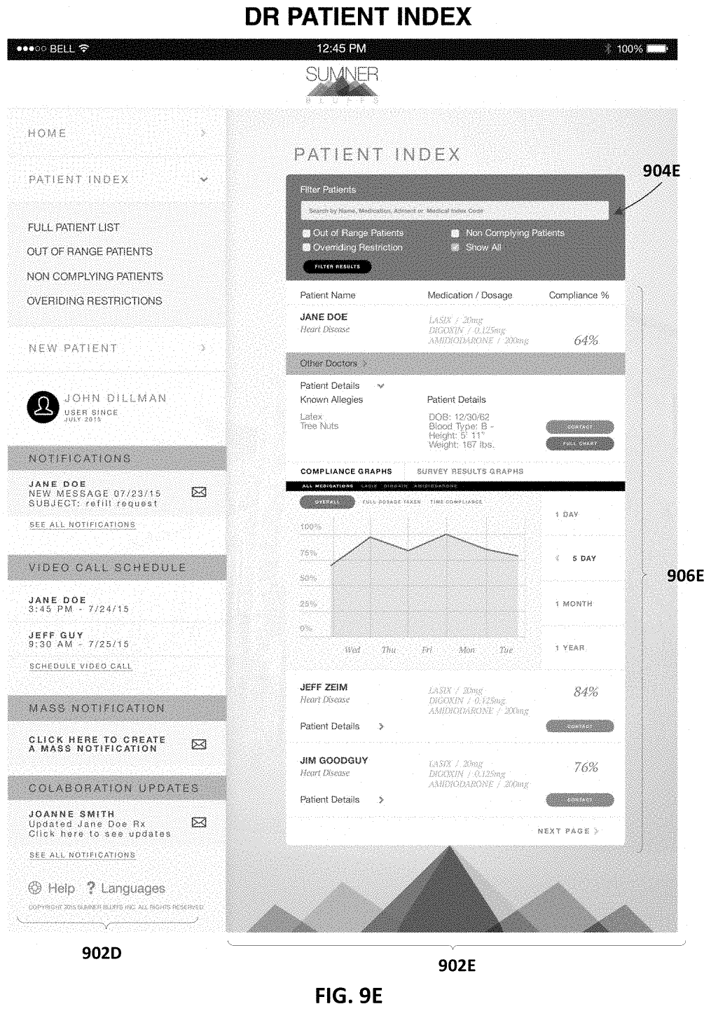

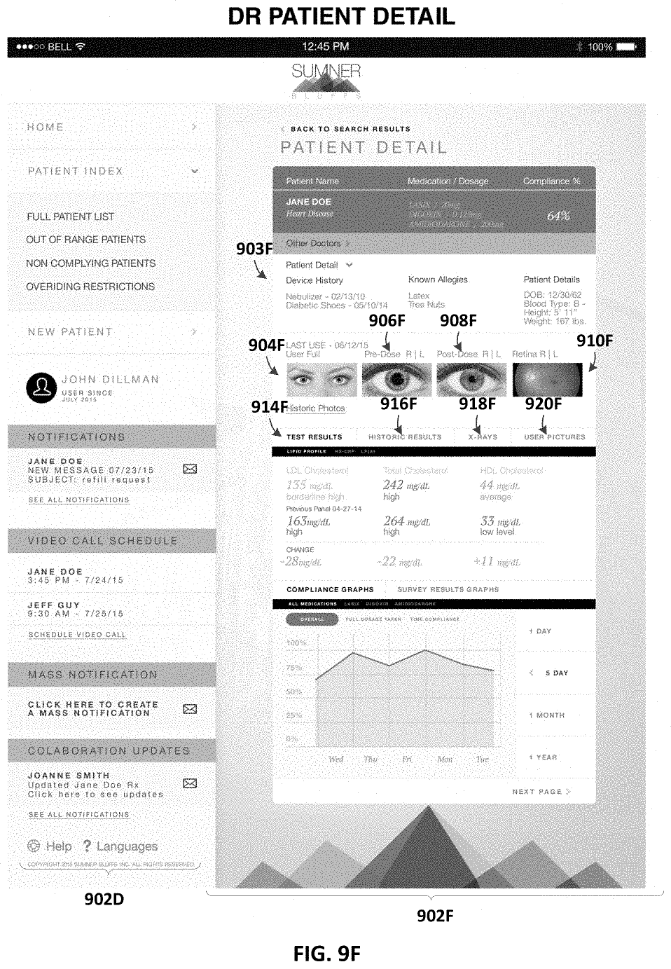

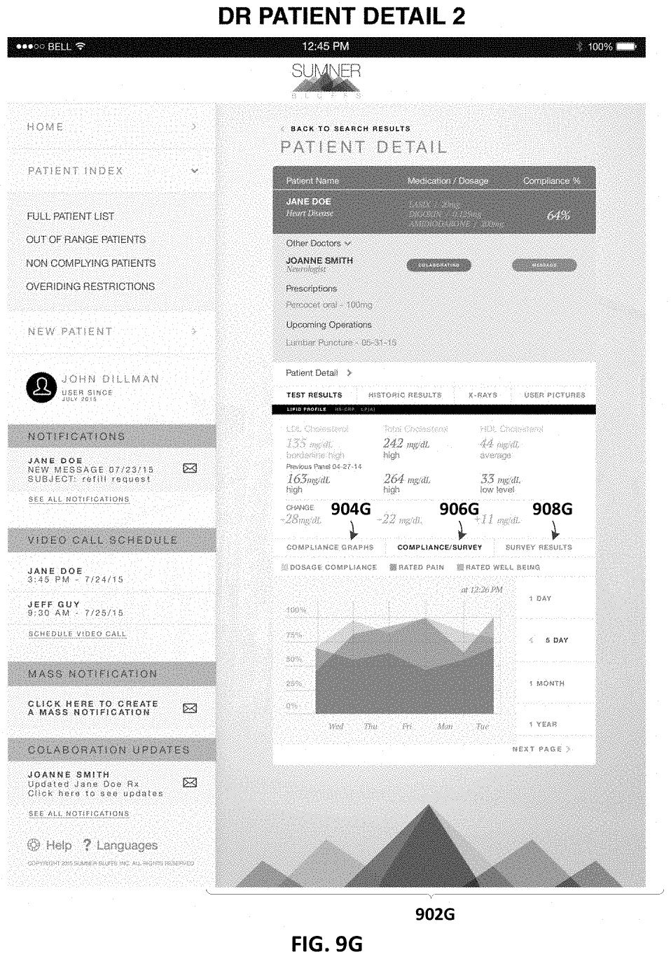

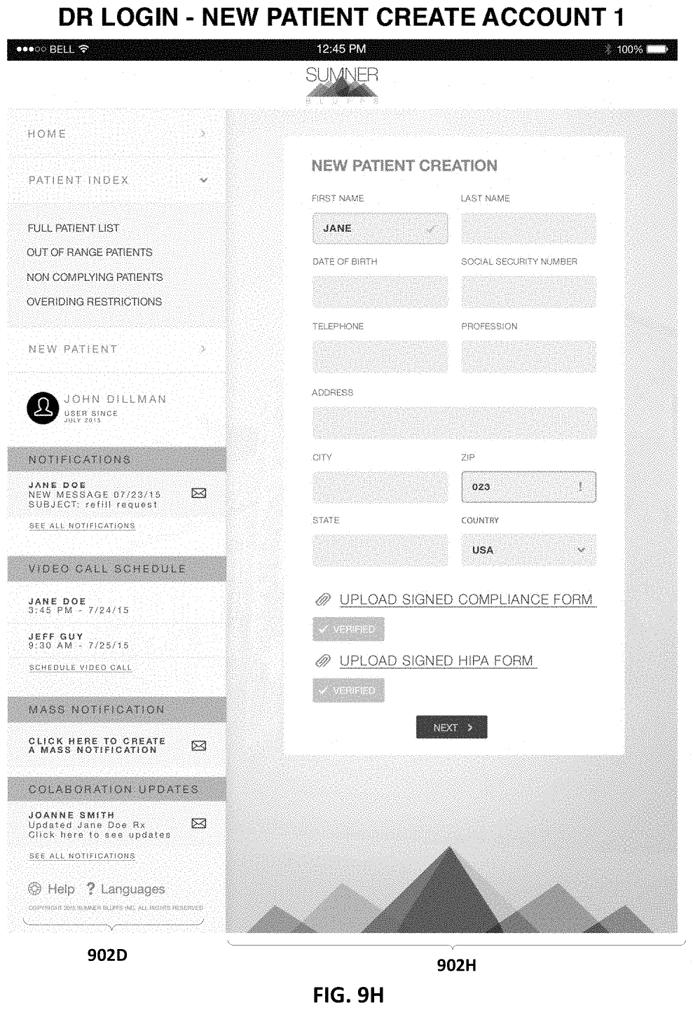

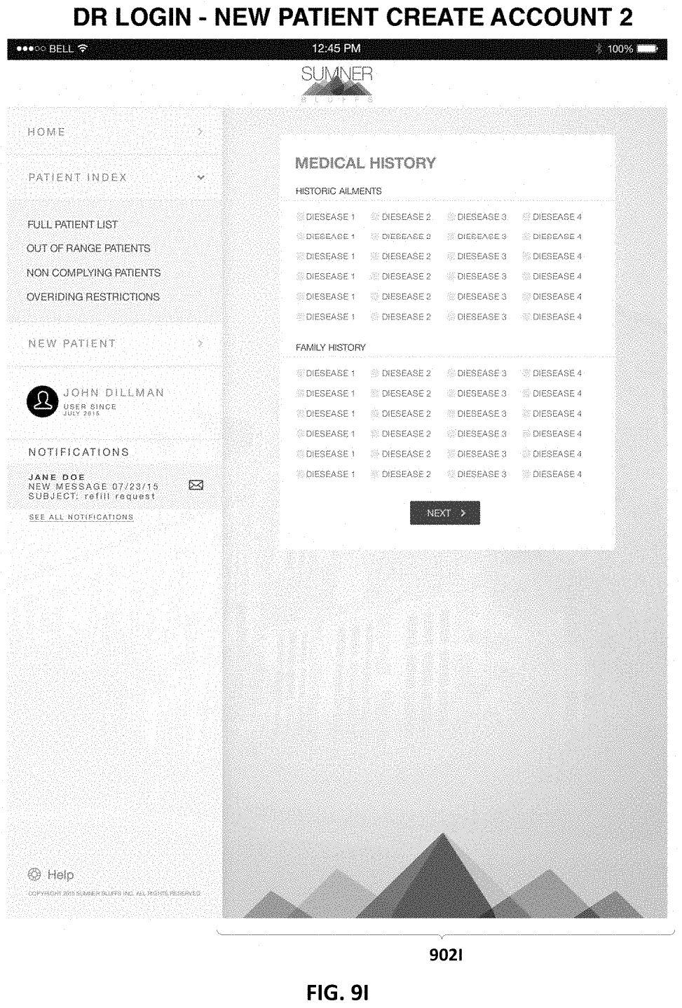

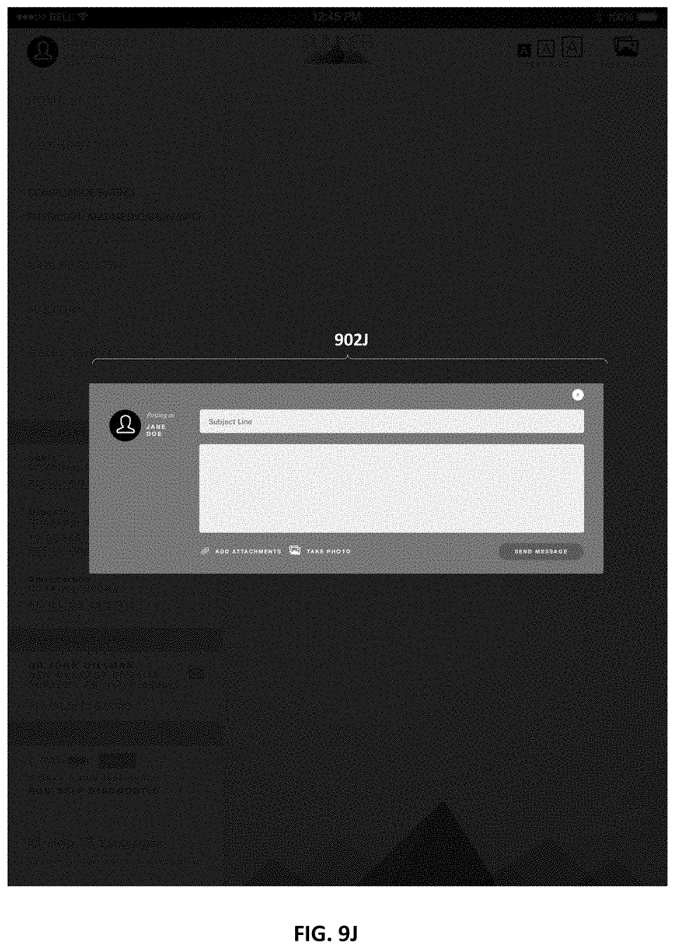

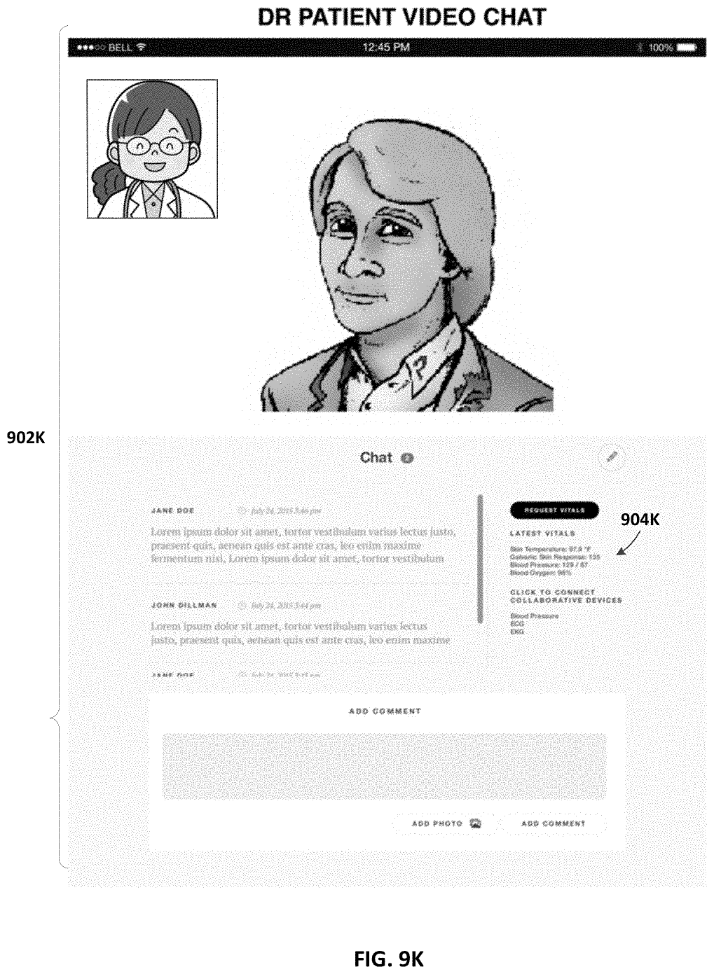

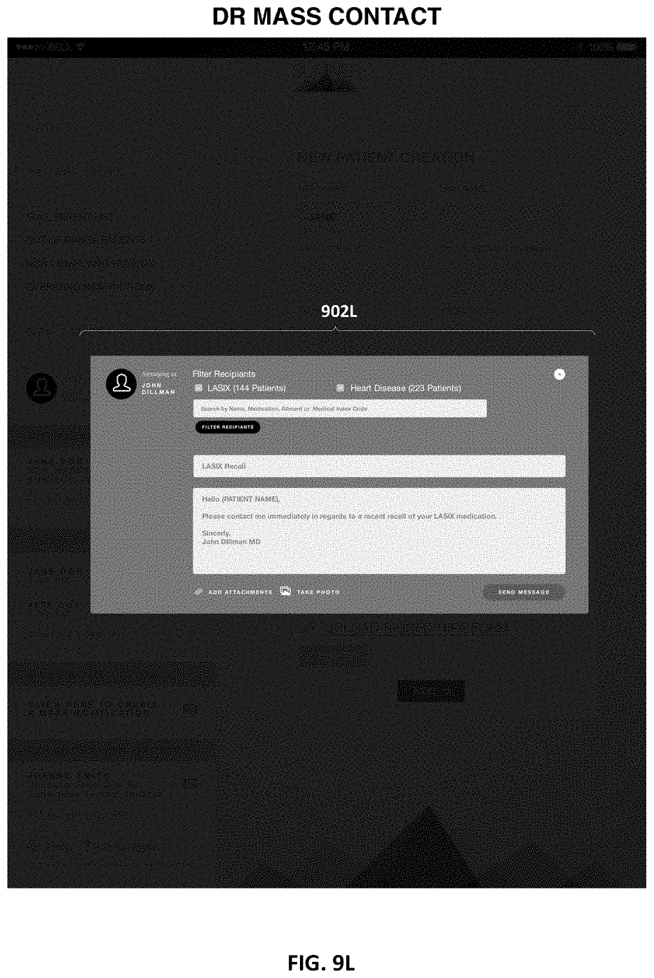

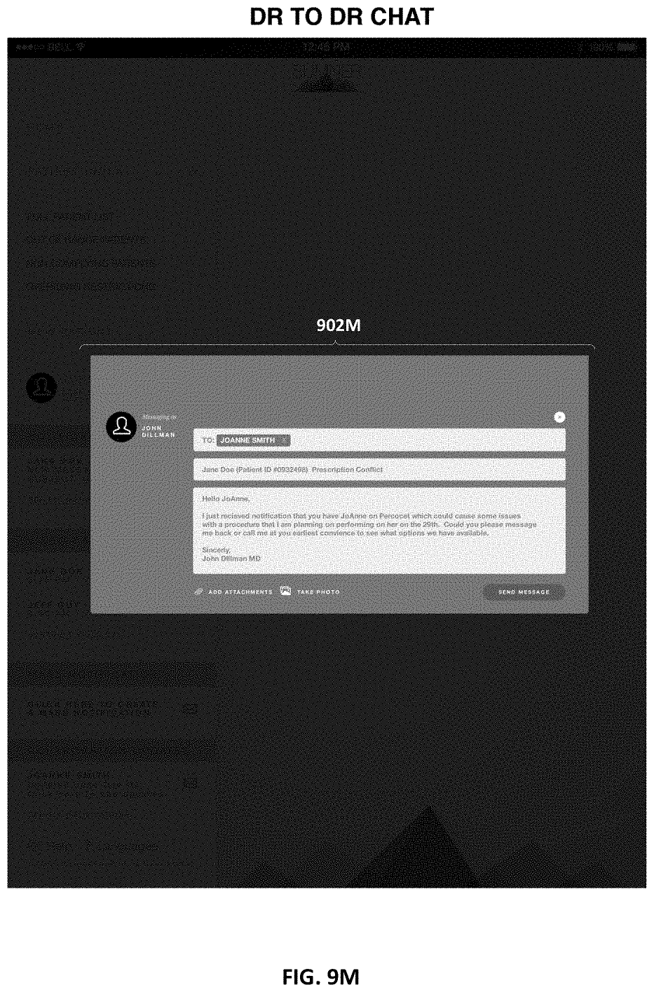

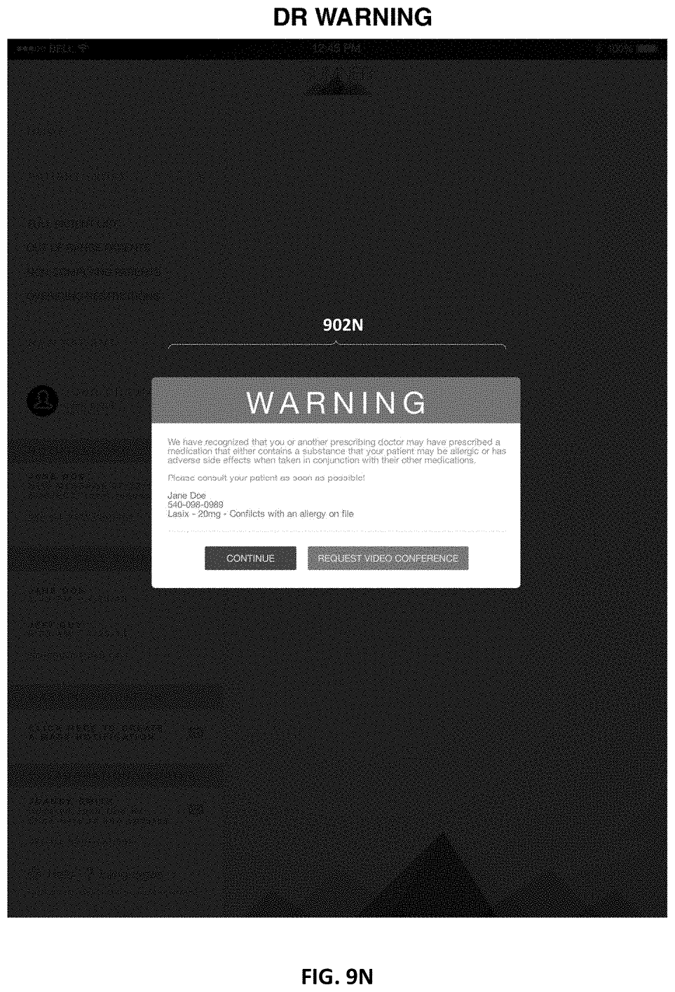

FIGS. 9A-9N illustrate certain embodiments of a third-party interface that implements the features and operations of FIG. 7.

For example, FIG. 9A represents an exemplary log in page 900A for a third-party. In some embodiments, the third-party can be a doctor that prescribes a pharmaceutical agent to a user. In the illustrated embodiments, the exemplary log in page 900A can include, but is not limited to, a sign up icon 902A that links to a sign-up page (see, e.g., FIGS. 9B and 9C). On the sign-up page, as illustrated in FIGS. 9B and 9C, a third-party can enter his/her personal information including, but not limited to, his/her name, email address, phone number, health system name, address, job title, professional license type, issuing state of license, license number, DEA number and national provider ID or other pertinent information. After the third-party creates an account, the third-party can enter his/her user name and password in the sign in fields 904A in order to sign in. If the third-party needs help signing in, the user can select a help icon 906A, which can direct them to a frequently asked questions (FAQs) page or other resource (e.g., a live chat) to help that third-party that is having issues signing in.

In other embodiments, once a third-party signs in, the third-party interface populates a page with a third-party sidebar 902D indicating various tabs that a third-party can select, as illustrated in FIG. 9D. The various tabs that populate the third-party sidebar 902D can include, but are not limited to, the following: a home tab 904D, a patient index tab 906D, a new patient tab 908D, a notifications tab 910D, a video call schedule tab 912D, a mass notification tab 914D, and a collaboration updates tab 916D.

In certain embodiments, a homepage 918D, as illustrated in FIG. 9D, can be displayed concurrently with the third-party sidebar 902D after the homepage tab 904D is selected. The homepage 918D can include an overall patient compliance record 920D. In some embodiments, the overall patient compliance record 920 can include components of a compliance record. For example, the following can be displayed: how many of his/her patients completed the survey, how many completed their dosage and how many users are complying with the time for taking their dosage. Alternatively or in addition to, the homepage 918D can include any notifications 922D from users that are provided to the third-party. In alternative embodiments, notifications 922D can be displayed when the third-party selects the notifications tab 910D.

In other embodiments, a patient index page 902E, as illustrated in FIG. 9E can be displayed concurrently with the third-party sidebar 902D after the patient index tab 906D is selected. The patient index page 902E can include, but is not limited to, a search bar 904E where the third-party can search for a user. The third-party can search for a user in the search bar 904E using various methods including, but not limited to, a user's name, the users that are out of range, the users that are not complying with their directed dosages, and the users that are overriding their restrictions. The results from a search results 906E of a search can be displayed below the search bar 904E.

From the search results 906E, the third-party can select a user. After a user is selected, information about the user can be displayed in patient detail pages 902F, 902G, as illustrated in FIGS. 9F, 9G. In certain embodiments, the user's personal information, the user's medication information 903F, the user's compliance rating 922F, the user's test results 914F, 916F, 918F, 920F, pictures 904F, 906F, 908F, 910F of the user and information downloaded from the pharmaceutical delivery and biometric monitoring device 100 can be displayed in the patient detail pages 902F, 902G. The data displayed on the patient detail pages 902F, 902G can be used by the third-party to determine whether the medication is effective for the user. The third-party can use this information and other patient information direct the user to maintain the current dosage of the pharmaceutical agent, direct the user decrease or increase the dosage of the pharmaceutical agent, or increase or decrease frequency of use, or direct the user to discontinue taking the pharmaceutical agent or direct the user to call a medical professional immediately if the user's test results indicate that the user needs immediate assistance.

Referring to FIG. 9F, in some embodiments, the patient detail page 902F can include various illustrations 904F, 906F, 908F, 910F for a third-party to review, as presented above. For example, the third-party can review a photo 904F of the user that is receiving the medication. This may be helpful in determining that the user directed to take the pharmaceutical agent matches the records. Other pictures the third-party can receive and review can include a pre-dose picture of the user's eyes 906F and a post-dose picture of the user's eyes 908F (both left and right eyes can be reviewed, even though only one eye is displayed). The third-party can use this information to determine the reaction of the user's eye to the pharmaceutical agent which may be helpful for additional diagnosis and/or agent responsiveness of the user. Other pictures the third-party can review can be the user's retinas 910F (again, both left and right retinas can be reviewed, even though only one retina is shown). This can assist the third party to determine a retinal response to the pharmaceutical agent of the user.

In other embodiments, the patient detail page 902F can also link to the user's medical records. The third-party can then review the user's medical records. The medical records can include, but is not limited to, the user's most recent test results 914F, the user's historic test results 916F, any x-rays 918F the user has had and user pictures 920F. The user pictures illustrated in 920F can be similar to the pictures 904F, 906F, 908F, 910F described above, but related to previous doses, not the most recent dose.

Referring to FIG. 9G, the patient detail page 902G can also include a compliance graph 904G. The compliance graph 904G can include, but is not limited to, icons for viewing the user's overall compliance rating, the user's full dosage compliance and the user's time compliance. A compliance survey graph 906G can also be included in the patient detail page 902G. The compliance survey graph 906G can include the user's dosage compliance, the user's rated pain and the user's rated well-being. In other embodiments, the patient detail page 902G can also include the user's survey results 908G. For each respective chart 904G, 906G, 908G, different time periods can be selected to display (e.g., 1 day, 2 days, 3 days, 4 days, 5 days, 1 week, 10 days, 1 month, 1 year, or other appropriate time period.).

In other embodiments, patient creation pages 902H, 902I, as illustrated in FIGS. 9H, 9I, can be displayed concurrently with the third-party sidebar 902D after the new patient tab 908D is selected. In some embodiments, the patient creation pages 902H, 902I can receive input from the third-party about the user. For example, information that the third-party can input to the system includes, but is not limited to, the user's name, date of birth, social security number, telephone number, profession, and address. Additionally, the third-party can input the medical history of the patient and any family medical history as appropriate, as illustrated in FIG. 9I.

In some embodiments, a message page 902J can be displayed if the third-party wants to respond to the notification message 922D, as illustrated in FIG. 8I. The user can type their message in the text box in the message page 902J and send the message to the person who originally sent the notification message 922D, as illustrated in FIG. 9J. In some embodiments, the message page 902J can be displayed after a user selects a messaging tab (not shown), if the user chooses to send a message to a third-party without having to respond to a notification message 922D.

In some embodiments, a video chat page 902K, as illustrated in FIG. 9K, can be used to respond to a notification message 922D. In some other embodiments, the video chat page 902L can be displayed after selecting a video chat tab (not shown) or video call schedule tab 912D. In some embodiments, a user can request a video chat with the third-party in a notification message 922D and the third-party can accept the video chat request, which will enable the video chat page 922D. In the video chat page 902K, the user's latest vitals 904K can be displayed for the third-party to review.

In some embodiments, a mass contact page 902L, can be displayed after the mass notifications tab 914D is selected, as shown in FIG. 9L. As an example, the third-party can send a message to all the users that they have set up an account for in the patient creation pages 904H, 902I. As another example, the third-party can send a message to all users that are taking a specific pharmaceutical agent or monitored agent. This may be helpful if additional information about a pharmaceutical agent is discovered and the third-party wishes to notify all the users that are taking a dosage of the pharmaceutical agent. As another example, the third-party may select specific users, from the users that they have set up an account for in the patient creation pages 904H, 902I, to send a notification.

In some embodiments, a third-party-to-third-party contact page 902M, can be displayed after the collaboration updates tab 916D is selected, as shown in FIG. 9M. This can be helpful if a user transfers from a first third-party to a second third-party to receive treatment. If the second third-party has questions about previous treatments for the user, the second third-party can contact the first third-party and vice-versa.

In some embodiments, a warning page 902N can be displayed in the third-party interface, as illustrated in FIG. 9N. In some embodiments, a warning page 902N can be displayed in the third-party interface when a third-party has directed a user to take a pharmaceutical agent that the user is allergic to or sensitive to for any reason and at any level (e.g. the user gets hives, vomits or airway obstruction for example). In some embodiments, a warning page 902N can be displayed when a third-party has directed a user to take a pharmaceutical agent that may have an adverse side effect when taken in conjunction with other pharmaceutical agents that the user is directed to take. In some embodiments, as illustrated in the warning page 902N, a description of the pharmaceutical agent that resulted in the warning can be illustrated in the warning page 902N. In some embodiments, the warning page 902N may include a button that allows a user to request a video conference with the third-party that directed the user to take the pharmaceutical agent.

As presented above, FIGS. 8A-9N are only examples and are not meant to be limiting.

### Exemplary Network Operating Environment

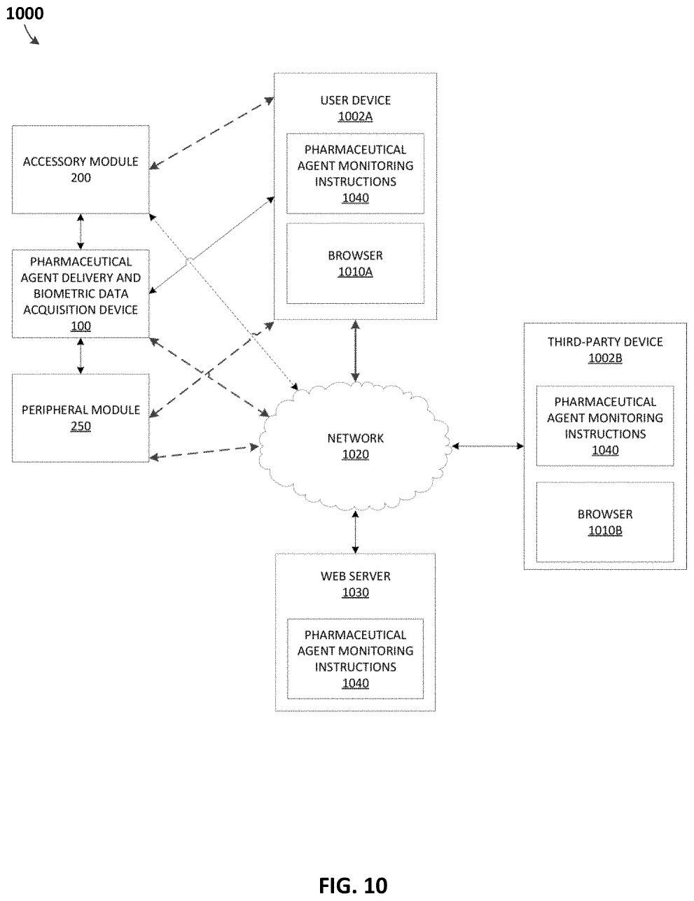

FIG. 10 is a block diagram of an exemplary network operating environment 1000 for computing devices (e.g., a user device 1002A and a third-party device 1002B) that implement features and operations of examples illustrated in FIGS. 7-9N. The terms computing device will be used interchangeably with the terms user device 1002A and third-party device 1002B. FIG. 10 uses like reference numbers to identify like elements (e.g., the pharmaceutical agent deliver and biometric data acquisition device 100 in FIG. 10 has the same characteristics and functionality as the pharmaceutical agent deliver and biometric data acquisition device 100 in FIGS. 1-4). A letter after a reference number, such as “1010A,” indicates that the text refers specifically to the element having that particular reference numeral. A reference numeral in the text without a following letter, such as “1010,” refers to any or all of the elements in the figures bearing that reference numeral (e.g., “1010” in the text refers to reference numerals “1010A” and/or “1010B” in the figures).

The pharmaceutical agent delivery and biometric data acquisition device 100, the accessory module 200, and the peripheral module 250 can have the same characteristics and functionality as described in FIGS. 1-4 above. Moreover, they can be communicatively coupled, using either a wireless or wired connection, as described above. The user device 1002A can be used by a patient for interacting with the pharmaceutical agent delivery and biometric data acquisition device 100. All of the data that the pharmaceutical agent delivery and biometric data acquisition device 100 acquires can be sent to the user device 1002A. In some embodiments, the user device 1002A can also be used by a patient for interacting with the accessory module 200 and peripheral module 250.

The user device 1002A, the third-party device 1002B and the web server 1030 can include pharmaceutical agent monitoring instructions 1040 which, when executed by a processing device, can perform the features and operations of FIGS. 7-9N. The user device 1002A and the third-party device 1002B are used by a patient and a third-party, respectively, and can be used for interacting with the pharmaceutical agent monitoring instructions 1040 stored on the web server 1030.

The user device 1002A and the third-party device 1002B can use respective browsers 1010A, 1010B to access one or more web pages or other web content presented by the web server 1030. Furthermore, the user device 1002A and the third-party device 1002B can provide data to, and receive data from, the pharmaceutical agent monitoring instructions 1040 located on the web server 1030. The term “data” can include, but is not limited to: patient surveys, responses to a patient surveys, patient health records and other patient related information, tests, administered tests, responses to administered tests, data pertaining to a pharmaceutical agent, prescribed parameters of a pharmaceutical agent, biometric parameters and responses, data from the pharmaceutical agent delivery and biometric data acquisition device 100, and data from the accessory and peripheral modules 200, 250.

In some embodiments, an application on the patient electronic device 1002A and/or the third-party electronic device 1002B can perform all of the processes of the pharmaceutical agent monitoring instructions 1040. In some other embodiments, the web server 1040 can perform all of the processes of the pharmaceutical agent monitoring instructions 1040. In yet other embodiments, one or more of the processes of the pharmaceutical agent monitoring instructions 1040 can be performed by the user device 1002A and third-party device 1002B and one or more of the processes of the pharmaceutical agent monitoring instructions 1040 can be performed by the web server 1040.

The pharmaceutical agent delivery and biometric data acquisition device 100, the accessory module 200, the peripheral module 250, the user device 1002A, the third-party device 1002B, and the web server 1030 can, for example, communicate over one or more wired and/or wireless networks 1020 in data communication. In some embodiments, the network 1020 is the Internet. The network 1020 can also utilize dedicated or private communication links (e.g., WAN, MAN, LAN) that are not necessarily part of the Internet. The network 1020 can use standard communications technologies and/or protocols.

As illustrated above, some aspects of the user matter of this specification can include gathering and use of data available from various sources to improve services a user device can provide to a user. The present disclosure contemplates that in some embodiments; gathered data may include, but is not limited to, personal information data that uniquely identifies or can be used to contact or locate a specific person. Such personal information data can include demographic data, location based data, telephone numbers, email addresses, Twitter™ ID's, home addresses, medical data, or any other identifying information.

The present disclosure recognizes that the use of such personal information data, in the present technology, can be used to the benefit of users. For example, the personal information data can be used to deliver updated medication information to the user. Further, other uses for personal information data that benefit the user are also contemplated by the present disclosure. It is noted that all authorized persons able to receive this information related to the user will have the ability to log-in and view the user's medical information as may be necessary for the authorized person (e.g. caregiver, family member) to view.

The present disclosure further contemplates that the entities responsible for the collection, analysis, disclosure, transfer, storage, or other use of such personal information data will comply with well-established privacy policies and/or privacy practices. For example, such entities should implement and consistently use privacy policies and practices that are generally recognized as meeting or exceeding industry or governmental requirements for a particular region for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

### Exemplary Computing Device Architecture

FIG. 11 is a block diagram illustrating an exemplary computing device architecture 1100 capable of implementing the features and operations of FIGS. 7-9N. A computing device (e.g., a user device 1002A or a third-party device 1002B as described in FIG. 10) can include a memory interface 1102, one or more data processors, image processors and/or processors 1104, and a peripherals interface 1106. Memory interface 1102, one or more processors 1104 and/or peripherals interface 1106 can be separate components or can be integrated in one or more integrated circuits. Processors 1104 can include application processors, baseband processors, and wireless processors. The various components in the mobile device, for example, can be coupled by one or more communication buses or signal lines.

Sensors, devices, and subsystems can be coupled to peripherals interface 1106 to facilitate multiple functionalities. For example, a motion sensor 1110, a light sensor 1112, and a proximity sensor 1114 can be coupled to the peripherals interface 1106 to facilitate orientation, lighting, and proximity functions of the mobile device. The motion sensor 1110 can include one or more accelerometers configured to determine change of speed and direction of movement of the mobile device. A location processor 1116 (e.g., GPS receiver) can be connected to the peripherals interface 1106 to provide geo-positioning. A magnetometer 1118 (e.g., an integrated circuit chip) can also be connected to the peripherals interface 1106 to provide data that can be used to determine the direction of magnetic North. As a result, the magnetometer 1118 can be used as an electronic compass. A barometer 1120 can be connected to the peripherals interface 1106 and be configured to measure pressure of atmosphere around the mobile device.

A camera subsystem 1122 can be coupled to the peripherals interface 1106. The camera subsystem 1122 can be coupled to an optical sensor (not shown), e.g., a charged coupled device (CCD) or a complementary metal-oxide semiconductor (CMOS) optical sensor, which can be utilized to facilitate camera functions, such as recording photographs and video clips.

Communication functions can be facilitated through one or more wireless communication subsystems 1124, which can include radio frequency receivers and transmitters and/or optical (e.g., infrared) receivers and transmitters. The specific design and implementation of the communication subsystem 1124 can depend on the communication network(s) over which a mobile device is intended to operate. For example, a mobile device can include communication subsystems 1124 designed to operate over a global system for mobile communications (“GSM”) network, a global packet radio service (“GPRS”) network, an enhanced data rates for GMS evolution (“EDGE”) network, a Wi-Fi™ or WiMAX™ network, and a Bluetooth™ network. In particular, the wireless communication subsystems 1124 can include hosting protocols such that the mobile device can be configured as a base station for other wireless devices.

An audio subsystem 1126 can be coupled to the peripherals interface 1106. The audio subsystem 1126 can also be coupled to a speaker (not shown) and a microphone (not shown) to facilitate voice-enabled functions, such as voice recordation (e.g. for a sight-impaired user), voice recognition, voice replication, digital recording, and telephony functions. The audio subsystem 1126 can be configured to receive voice commands from the user.

An Input/Output (I/O) subsystem 1128 can be coupled to the peripherals interface 1106. The I/O subsystem can include a touch surface controller 1130 and/or other input controller(s) 1134. The touch surface controller 1130 can be coupled to a touch surface 1132 or pad. The touch surface 1130 can include, for example, a touch screen. The touch surface 1132 and touch surface controller 1130 can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with touch surface 1132.

The other input controller(s) 1134 can be coupled to other input/control devices 1136, such as one or more buttons, rocker switches, thumb-wheel, infrared port, USB port, and/or a pointer device such as a stylus. The one or more buttons (not shown) can include an up/down button for volume control of speaker (not shown) and/or microphone (not shown).

In one implementation, a pressing of the button for a first duration may disengage a lock of the touch surface 1132; and a pressing of the button for a second duration that is longer than the first duration may turn power to the mobile device on or off. The user may be able to customize a functionality of one or more of the buttons. The touch surface 1132 can, for example, also be used to implement virtual or soft buttons and/or a keyboard.

The memory interface 1102 can be coupled to memory 1140. The memory 1140 can include high-speed random access memory and/or non-volatile memory, such as one or more magnetic disk storage devices, one or more optical storage devices, and/or flash memory (e.g., NAND, NOR). The memory 1140 can store operating system 1153, such as Darwin, RTXC, LINUX, UNIX, OS X, WINDOWS, iOS, or an embedded operating system such as VxWorks. The operating system 1153 may include instructions for handling basic system services and for performing hardware dependent tasks. In some implementations, the operating system 1153 can include a kernel (e.g., UNIX kernel).

The memory 1140 may also store communication instructions 1152 to facilitate communicating with one or more additional devices, one or more computers and/or one or more servers. The memory 1140 may include graphical user interface (GUI) instructions 1151 to facilitate graphic user interface processing; sensor processing instructions 1150 to facilitate sensor related processing and functions; phone instructions 1149 to facilitate phone-related processes and functions; electronic messaging instructions 1148 to facilitate electronic-messaging related processes and functions; web browsing instructions 1147 to facilitate web browsing-related processes and functions; media processing instructions 1146 to facilitate media processing-related processes and functions; GPS/Navigation instructions 1145 to facilitate GPS and navigation-related processes and instructions; camera instructions 1144 to facilitate camera-related processes and functions; magnetometer data 1143 and calibration instructions 1142 to facilitate magnetometer calibration. The memory 1140 may also store other software instructions (not shown), such as security instructions, web video instructions to facilitate web video-related processes and functions, and/or web shopping instructions to facilitate web shopping-related processes and functions. In some implementations, the media processing instructions 1146 are divided into audio processing instructions and video processing instructions to facilitate audio processing-related processes and functions and video processing-related processes and functions, respectively. An activation record and International Mobile Equipment Identity (IMEI) or similar hardware identifier can also be stored in the memory 1140. The memory 1140 can store pharmaceutical agent monitoring instructions 1141. The pharmaceutical agent monitoring instructions 1141, upon execution, can cause the processor 1104 to perform the operations of method 700 as described above in reference to FIG. 7.

Each of the above identified instructions and applications can correspond to a set of instructions for performing one or more functions described above. These instructions need not be implemented as separate software programs, procedures, or modules. The memory 1140 can include additional instructions or fewer instructions. Furthermore, various functions of the mobile device may be implemented in hardware and/or in software, including in one or more signal processing and/or application specific integrated circuits.

### Exemplary Web Server Architecture

FIG. 12 is a block diagram of an exemplary web server architecture 1200 for implementing the features and operations of FIGS. 7-9N.

In embodiments, the web server architecture 1200 may also be used to help detect fraud. In embodiments, the fraud detected may be fraud committed by a hospital, a doctor and/or an individual. In the example of a hospital, a hospital may be performing a procedure too often and unnecessarily. In the example of a doctor, a doctor may be performing a procedure too often or prescribing a pharmaceutical agent too often. In the example of an individual, an individual may be visiting multiple doctors and receiving multiple doses of a pharmaceutical agent to treat the same disorder.

In embodiments, fraud may be determined by the web server architecture 1200 based on correlations computed by the web server architecture 1200. For example, the web server architecture 1200 may track international classification of diseases (ICD) codes, which may be entered by the individual or third-party into the user and/or third-party interfaces described in FIGS. 8A-9N; or, the ICD code may be retrieved directly from different hospitals, as stated in FIG. 8O above. Using the ICD codes, the web server architecture 1200 may determine whether any hospitals and/or doctors are outliers for various procedures. That is, for example, a certain procedure that is performed by a hospital and/or doctor may be at a significantly higher rate than other hospitals, after adjusting for the amount of patients seen by the hospital and/or doctor. As such, the outliers may indicate that a procedure may be being performed too often to, perhaps, receive a federal Medicare payment. If this situation is identified by the web server architecture 1200, the web server architecture 1200 may report the outlier to a proper authority. Similarly, the ICD codes may be used to determine whether a user may be possibly committing fraud by visiting multiple doctors and complaining of the same ailment, in order to, perhaps, receive an abundance of pharmaceutical agents. In embodiments, the web server architecture 1200 may also correlate how often the doctor is prescribing a certain pharmaceutical agent and/or performing a procedure.

Other architectures are possible, including architectures with more or fewer components. The web server architecture 1200 can be implemented by web server 1030 in FIG. 10. In some embodiments, Web Server Architecture 1200 includes one or more processors 1202, one or more output devices 1204, one or more network interfaces 1206, one or more input devices 1208 and one or more computer readable mediums 1212. These components can exchange communications and data over one or more communication channels 1210 which can utilize various hardware and software for facilitating the transfer of data and control signals between components.

The term “computer-readable medium” refers to any medium that participates in providing instructions to processor 1202 for execution, including without limitation, non-volatile media (e.g., optical or magnetic disks), volatile media (e.g., memory) and transmission media. Transmission media includes, without limitation, coaxial cables, copper wire and fiber optics.

The computer-readable medium 1212 can further include operating system 1214 (e.g., Mac OS® server, Windows Server®, or iOS®), network communication module 1216, and pharmaceutical agent monitoring instructions 1218. The operating system 1214 can be multi-user, multiprocessing, multitasking, multithreading, real time, etc. The operating system 1214 performs basic tasks, including but not limited to: recognizing input from and providing output to devices 1206, 1208; keeping track and managing files and directories on computer-readable mediums 1212 (e.g., memory or a storage device); controlling peripheral devices; and managing traffic on the one or more communication channels 1210. The network communications module 1216 includes various components for establishing and maintaining network connections (e.g., software for implementing communication protocols, such as TCP /IP, HTTP, etc.). The pharmaceutical agent monitoring instructions 1218 can include instructions that, when executed, causes the processor 1202 to perform the operations of method 700 as described above in reference to FIG. 7.

The web server architecture 1200 can be implemented in a parallel processing or peer-to-peer infrastructure or on a single device with one or more processors. Software can include multiple software components or can be a single body of code.

The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language (e.g., Swift, Objective-C, Java), including compiled or interpreted languages, and it can be deployed in any form, including as a stand-alone program or as a module, component, subroutine, a browser-based web application, or other unit suitable for use in a computing environment.

Suitable processors for the execution of a program of instructions include, by way of example, both general and special purpose microprocessors, and the sole processor or one of multiple processors or cores, of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, application specific integrated circuits (ASICs).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, and the computers and networks forming the Internet.

The computer system can include clients and servers. A client and server are generally remote from each other and typically interact through a network. The relationship of client and server arises by virtue of computer programs running on the respective computers and having a client-server relationship to each other.

A number of variations and modifications of the disclosure can be used. It would be possible to provide for some features of the disclosure without providing others.

The present disclosure, in various aspects, embodiments, and configurations, includes components, methods, processes, systems and/or apparatus substantially as depicted and described herein, including various aspects, embodiments, configurations, sub combinations, and subsets thereof. Those of skill in the art will understand how to make and use the various aspects, aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, e.g., for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable user matter.

What is claimed is:

1. A method comprising:
receiving, from a pharmaceutical agent delivery and biometric data acquisition device, delivery parameters of an administered pharmaceutical agent to a user;
receiving, from the pharmaceutical agent delivery and biometric data acquisition device, at least one biometric response of the user; and
determining, using a computing device, a compliance rating using at least one of the following: the delivery parameters of the administered pharmaceutical agent and the at least one biometric response.

2. The method according to claim 1, wherein the at least one biometric response includes at least one of the following: a galvanic skin response, a blood oxygen level response, a body temperature response, a heartrate response, a perfusion index response, a blood pressure response, a retina response, an eye movement response, an inhalation velocity response, an inhalation pressure response, an inhalation volume response, an expiratory velocity response, an expiratory pressure response, an expiratory volume response or an exhale chemical composition response.

3. The method according to claim 1, wherein the at least one biometric response to the pharmaceutical agent is measured by the pharmaceutical agent delivery and biometric data acquisition device during at least one of the following intervals: less than five minutes after taking the pharmaceutical agent, less than an hour after taking the pharmaceutical agent, less than a day after taking the pharmaceutical agent, less than a week after taking the pharmaceutical agent or less than a month after taking the pharmaceutical agent.

4. The method according to claim 1, further comprising receiving at least one biometric parameter for a user, wherein the at least one biometric parameter for the user is measured by the pharmaceutical agent delivery and biometric data acquisition device before the pharmaceutical agent is administered to the user, wherein the at least one biometric response and the at least one biometric parameter are used for revising the delivery parameter of the pharmaceutical agent.

5. The method according to claim 4, wherein the at least one biometric parameter includes at least one of the following: blood oxygen level, body temperature, heartrate, perfusion index, blood pressure, inhalation velocity, inhalation pressure, inhalation volume, expiratory velocity, expiratory pressure, expiratory volume or exhale chemical composition.

6. The method according to claim 1, further comprising:
determining whether the at least one biometric response for the user is within a range; and
sending an alert to at least one of the user or a third-party if the at least one biometric response is not within the range.

7. The method according to claim 1, further comprising determining, using a computing device, a response rating using the at least one biometric response.

8. (canceled)

9. The method according to claim 1, further comprising:
administering a test to the user;
receiving at least one response to the test; and
wherein the at least one received response for the test is used in determining the response rating.

10. The method according to claim 1, further comprising:
receiving a health record for the user; and
wherein the health record is used in determining the response rating.

11. The method according to claim 1, further comprising: receiving data from at least one peripheral device; and
wherein the data received from the at least one peripheral device is used in determining the response rating.

12. (canceled)

13. The method according to claim 1, wherein the pharmaceutical agent is at least one of the following: albuterol, albuterol sulfate, atropine sulfate, beclomethasone dipropionate, bitolterol mesylate, budesonide, formoterol fumarate, cromolyn sodium, desflurane, dexamethasone sodium phosphate, dornase alfa, enflurane, epinephrine, ergotamine tartrate, flunisolide, fluticasone propionate, fomoterol fumarate, halothane, iloprost, insulin, ipratropium bromide, isoetharine hydrochloride, isoflurane, isoproterenol hydrochloride, levalbuterol hydrochloride, metaproterenol sulfate, methacholine chloride, mometasone furoate, nedocromil sodium, nicotine, nitric oxide, pentamidine isethionate, pentetate calcium trisodium, pentetate zinc trisodium, pirbuterol acetate, ribavirin, salmeterol xinafoate, sevoflurane, tetrahydrocannabinol, tiotropium bromide monohydrate, tobramycin, trimcinolone acetonide, zanamivir, and combinations and derivatives thereof.

14. An apparatus comprising:
a computing device; and
a pharmaceutical agent monitoring module executed by the computing device and configured to:
receive, from a pharmaceutical agent delivery and biometric data acquisition device, delivery parameters of an administered pharmaceutical agent to a user;
receive, from the pharmaceutical agent delivery and biometric data acquisition device, at least one biometric response of the user; and
determine, using a computing device, a compliance rating using at least one of the following: the delivery parameters of the administered pharmaceutical agent and the at least one biometric response.

15. The apparatus according to claim 14, wherein the at least one biometric response includes at least one of the following: a galvanic skin response, a blood oxygen level response, a body temperature response, a heartrate response, a perfusion index response, a blood pressure response, a retina response, an eye movement response, an inhalation velocity response, an inhalation pressure response, an inhalation volume response, an expiratory velocity response, an expiratory pressure response, an expiratory volume response or an exhale chemical composition response.

16. The apparatus according to any one of claims 14-15, wherein the at least one biometric response to the pharmaceutical agent is measured by the pharmaceutical agent delivery and biometric data acquisition device during at least one of the following intervals: less than five minutes after taking the pharmaceutical agent, less than hour after taking the pharmaceutical agent, less than a day after taking the pharmaceutical agent, less than a week after taking the pharmaceutical agent or less than a month after taking the pharmaceutical agent.

17. The apparatus according to any one of claims 14-16, wherein the biometric response alert module is further configured to: receive at least one biometric parameter for a user, wherein the at least one biometric parameter for the user is measured by the pharmaceutical agent delivery and biometric data acquisition device before the pharmaceutical agent is administered to the user, wherein the at least one biometric response and the at least one biometric parameter are used for revising the delivery parameter of the pharmaceutical agent.

18. The apparatus according to claim 17, wherein the at least one biometric parameter includes at least one of the following: blood oxygen level, body temperature, heartrate, perfusion index, blood pressure, inhalation velocity, inhalation pressure, inhalation volume, expiratory velocity, expiratory pressure, expiratory volume or exhale chemical composition.

19-25.

26. The apparatus according to any one of claims 14-25, wherein the pharmaceutical agent is at least one of the following: albuterol, albuterol sulfate, atropine sulfate, beclomethasone dipropionate, bitolterol mesylate, budesonide, formoterol fumarate, cromolyn sodium, desflurane, dexamethasone sodium phosphate, dornase alfa, enflurane, epinephrine, ergotamine tartrate, flunisolide, fluticasone propionate, fomoterol fumarate, halothane, iloprost, insulin, ipratropium bromide, isoetharine hydrochloride, isoflurane, isoproterenol hydrochloride, levalbuterol hydrochloride, metaproterenol sulfate, methacholine chloride, mometasone furoate, nedocromil sodium, nicotine, nitric oxide, pentamidine isethionate, pentetate calcium trisodium, pentetate zinc trisodium, pirbuterol acetate, ribavirin, salmeterol xinafoate, sevoflurane, tetrahydrocannabinol, tiotropium bromide monohydrate, tobramycin, trimcinolone acetonide, zanamivir, and combinations and derivatives thereof.

27. A computer program product comprising a non-transitory computer readable storage medium containing program code, the computer program code when executed by a processor causes the processor to:
receive, from a pharmaceutical agent delivery and biometric data acquisition device, delivery parameters of an administered pharmaceutical agent to a user;
receive, from the pharmaceutical agent delivery and biometric data acquisition device, at least one biometric response of the user; and
determine, using a computing device, a compliance rating using at least one of the following: the delivery parameters of the administered pharmaceutical agent and the at least one biometric response.

28. The computer program product according to claim 27, wherein the at least one biometric response includes at least one of the following: a galvanic skin response, a blood oxygen level response, a body temperature response, a heartrate response, a perfusion index response, a blood pressure response, a retina response, an eye movement response, an inhalation velocity response, an inhalation pressure response, an inhalation volume response, an expiratory velocity response, an expiratory pressure response, an expiratory volume response or an exhale chemical composition response.

29. The computer program product according to any one of claims 27-28, wherein the at least one biometric response to the pharmaceutical agent is measured by the pharmaceutical agent delivery and biometric data acquisition device during at least one of the following intervals: less than five minutes after taking the pharmaceutical agent, less than hour after taking the pharmaceutical agent, less than a day after taking the pharmaceutical agent, less than a week after taking the pharmaceutical agent or less than a month after taking the pharmaceutical agent.

30-40. (canceled)
